(19) United States
(12) Patent Application Publication
(10) Pub. No.: US 2011/0200987 A1
(43) Pub. Date: Aug. 18, 2011

(54) COMPOSITIONS AND METHODS FOR MODIFYING CELLULAR PROPERTIES

(75) Inventors: Joseph Shiloach, Rockville, MD (US); Pratik Jaluria, Trumbull, CT (US); Michael Betenbaugh, Baltimore, MD (US)

(73) Assignees: Government of the United States of America, as represented by the Secretary, Department of Health, Rockville, MD (US); The John Hopkins University, Baltimore, MD (US)

(21) Appl. No.: 12/438,706

(22) PCT Filed: Aug. 24, 2007
(86) PCT No.: PCT/US07/18699

§ 371 Date: Mar. 16, 2011

Related U.S. Application Data
(60) Provisional application No. 60/840,381, filed on Aug. 24, 2006.
(60) Provisional application No. 60/931,439, filed on May 23, 2007.

(51) Int. Cl. C12Q 1/68; C12N 5/00; C12N 15/00; C12N 15/63; C07H 21/00

(52) U.S. Cl. 435/6.1; 435/375; 435/440; 435/320.1; 536/24.5; 435/325

(57) ABSTRACT

The invention generally provides compositions and methods for modulating cell properties and enhancing recombinant protein yields. In particular, the compositions and methods provide for cells having altered growth characteristics, including altered adhesion, rate of proliferation, growth to particular cell density, and recombinant protein expression level.

### CROSS REFERENCE TO RELATED APPLICATIONS

This application claims the benefit of the following U.S. Provisional Application No. 60/931,439, which was filed on May 23, 2007, and 60/840,381, which was filed on Aug. 24, 2006, the entire disclosures of which are hereby incorporated in its entirety.

### STATEMENT OF RIGHTS TO INVENTIONS MADE UNDER FEDERALLY SPONSORED RESEARCH

This work was supported by funding from the U.S. Government. Accordingly, the government has certain rights in the invention.

### BACKGROUND OF THE INVENTION

Cells engineered to produce biologically active products, such as antibodies or glycosylated products, are notoriously slow growing. Constraints on the ability to culture large numbers of cells at high density limits the amount of protein that can be produced in a given amount of time, and adds to the cost of biological products. One important consideration in choosing a cell line for therapeutic biological production is the adhesive characteristics of the cell, which may influence the cells ability to grow to high density and produce a biologically active product. For certain applications, anchorage-independent cell lines may be preferred, whereas for other applications, a cell line that adheres to a surface, e.g. is anchorage-dependent, may be preferable. The growth characteristics of the cells affect the production of recombinant proteins and therapeutic biologicals. Methods that enhance the growth of such cells, facilitate the production of recombinant polypeptides and therapeutic biologicals, and reduce associated costs are urgently required.

### SUMMARY OF THE INVENTION

The invention generally provides methods and compositions for modulating cell properties and enhancing recombinant protein yields by altering polynucleotide or polypeptide expressed in a cell.

In one aspect, the invention generally provides a method for identifying a gene whose expression modulates a cellular growth characteristic (e.g., cellular adhesion, cell growth or proliferation, cell death or mortality) the method involving comparing the expression of a gene in an anchorage-dependent cell relative to an anchorage-independent cell (e.g., mammalian cell; and identifying a gene that is differentially expressed in the anchorage-dependent cell relative to the anchorage-independent cell, wherein an alteration in the level of gene expression between the cells identifies a gene whose expression modulates cellular adhesion. In one embodiment, the expression of the gene is increased or decreased (e.g., by at least 5%, 10%, 25%, 50%, 75% or more) in the anchorage-dependent or the anchorage-independent cell. In another embodiment, the method further includes the steps of altering the expression of the gene in the cell, and comparing the adhesion, proliferation, mortality, or other growth characteristic of the cell relative to a corresponding control cell. In another embodiment, the cell is a cell in vivo or in vitro (e.g., a human cell in vitro). In another embodiment, gene expression is compared using a microarray. In yet another embodiment, the gene is any one or more of siat7e, lama4, cdkl3, cox15, egr1, and gas6.

In another aspect, the invention features a method for modulating a cell growth characteristic (e.g., cellular adhesion, cell growth or proliferation, cell death or mortality), the method involving contacting the cell with an agent that increases or decreases the expression of a gene (e.g., cdkl3, siat7e, lama4, cox15, egr1, gas6, map3k9, and gap43) identified according to the previous aspect, thereby modulating a cell growth characteristic. In one embodiment, the method involves contacting the cell with an expression vector that encodes any one or more of cdkl3, siat7e, lama4, cox15, egr1, gas6, map3k9, and gap43. In another embodiment, the method involves contacting the cell with an inhibitory nucleic acid molecule or an expression vector that encodes an inhibitory nucleic acid molecule that reduces the expression of any one or more of cdkl3, siat7e, lama4, cox15, egr1, gas6, map3k9, and gap43

In another aspect, the invention provides a method for modulating recombinant polypeptide expression in a cell, the method involving contacting a cell that expresses a recombinant polypeptide with an agent that increases or decreases the expression of a gene identified according to the first aspect or a gene selected from any one or more of cdkl3, siat7e, lama4, cox15, egr1, gas6, map3k9, and gap43 in a cell, thereby modulating expression of the recombinant polypeptide. In some embodiments, the recombinant polypeptide is a therapeutic polypeptide (e.g., an antibody, cytokine, growth factor, enzyme, immunomodulator, or thrombolytic).

In another aspect, the invention provides a method for modulating cellular mortality, the method comprising contacting the cell with an agent that increases or decreases the expression of a gene of the invention (e.g., egr1, gas6) or a gene identified according to the method of a previous aspect in a cell, thereby modulating cellular mortality.

In another aspect, the invention provides a method for modulating cellular proliferation, the method involving contacting the cell with an agent that increases or decreases the expression of a gene of the invention (e.g., cox15 or cdkl3) or a gene identified according to the method of a previous aspect in a cell, thereby modulating cellular proliferation.

In yet another aspect, the invention provides a method for increasing cell adhesion, the method involving contacting a cell with an expression vector containing a nucleic acid molecule that encodes lamanin α4; and increasing lamanin α4 expression in the cell, thereby increasing cell adhesion. In one embodiment, the method increases laminin α4 transcription or translation in the cell. In another embodiment, the expression vector comprises a constitutive promoter (e.g., CMV promoter) or a conditional promoter operably linked to the nucleic acid molecule.

In yet another aspect, the invention provides a method for decreasing cell adhesion, the method involving contacting a cell expressing lamanin α4 with an agent that decreases lamanin α4 expression or activity in the cell, thereby decreasing cell adhesion. In one embodiment, the agent is a lamanin α4 inhibitory nucleic acid molecule. In another embodiment, the lamanin α4 inhibitory nucleic acid molecule is a short interfering RNA (siRNA), antisense RNA, or short hairpin RNA.

In yet another aspect, the invention provides a method for decreasing cell adhesion, the method involving contacting a cell with an expression vector containing a nucleic acid molecule that encodes sialyltransferase 7E; and increasing sialyltransferase 7E expression in the cell, thereby decreasing cell adhesion. In yet another aspect, the method increases the transcription or translation of a sialyltransferase 7E nucleic acid molecule in the cell. In one embodiment, the expression vector comprises a constitutive or conditional promoter operably linked to a sialyltransferase 7E nucleic acid molecule. In another embodiment, the constitutive promoter is the CMV promoter.

In yet another aspect, the invention features a method for increasing cell adhesion, the method involving contacting a cell expressing sialyltransferase 7E with an agent that decreases sialyltransferase 7E expression or activity in the cell, thereby increasing cell adhesion. In one embodiment, the agent is a sialyltransferase 7E inhibitory nucleic acid molecule. In yet another embodiment, the inhibitory nucleic acid molecule is an short interfering RNA (siRNA), antisense RNA, or short hairpin RNA.

In yet another aspect, the invention features a method for increasing the expression of a recombinant polypeptide in a cell, the method involving contacting a cell that expresses a recombinant polypeptide with an expression vector containing a nucleic acid molecule or an inhibitory nucleic acid molecule selected from the group consisting of cdkl3, siat7e, lama4, cox15, egr1, gas6, map3k9, and gap43; and altering expression of the nucleic acid molecule in the cell, thereby increasing the expression of the recombinant polypeptide in the cell. In one embodiment, the polypeptide is a therapeutic polypeptide that is any one or more of an antibody, cytokine, growth factor, enzyme, immunomodulator, and thrombolytic.

In yet another aspect, the invention provides a method for increasing the expression of a recombinant polypeptide in a cell, the method involving contacting a cell that expresses a recombinant polypeptide with an expression vector containing a nucleic acid molecule encoding a polylaminin α4 or sialyltransferase 7E; and increasing laminin α4 or sialyltransferase 7E expression in the cell, thereby increasing the expression of the recombinant polypeptide in the cell.

A method for altering the growth characteristics of a cell, the method involving contacting the cell with an expression vector encoding a polypeptide or an inhibitory nucleic acid molecule that is any one or more of cdkl3, siat7e, lama4, cox15, egr1, gas6, map3k9, and gap43 polypeptide; and expressing the polypeptide in the cell, thereby altering the cell's growth characteristics. In one embodiment, the method increases cell proliferation, decreases cell adhesion, or decreases cell mortality.

In another aspect, the invention provides an expression vector containing a nucleic acid molecule encoding a polypeptide that is any one or more of cdkl3, siat7e, lama4, cox15, egr1, gas6, map3k9, and gap43. In one embodiment, the polypeptide is operably linked to a promoter that directs expression of the nucleic acid molecule in a mammalian cell. In another embodiment, the promoter is a constitutive or inducible promoter.

In yet another aspect, the invention provides an expression vector containing a nucleic acid molecule encoding an inhibitory nucleic acid molecule complementary to a gene selected from the group consisting of cdkl3, siat7e, lama4, cox15, egr1, gas6, map3k9, and gap43.

In still another aspect, the invention provides an inhibitory nucleic acid molecule that reduces the expression of a nucleic acid molecule selected from the group consisting of cdkl3, siat7e, lama4, cox15, egr1, gas6, map3k9, and gap43.

In various embodiments of the previous aspects, the inhibitory nucleic acid molecule is a short interfering RNA, antisense oligonucleotide, or short hairpin RNA.

In another aspect, the invention provides a cell containing an expression vector, where the vector contains a nucleic acid molecule encoding a polypeptide that is any one or more of cdkl3, siat7e, lama4, cox15, egr1, gas6, map3k9, and gap43. In one embodiment, the cell expresses an increased level of a siat7e, lama4, cdkl3, cox15, egr1, or gas6 nucleic acid molecule or polypeptide relative to a control cell.

In yet another aspect, the invention provides a cell containing an expression vector containing a nucleic acid molecule encoding a siat7e, lama4, cdkl3, cox15, egr1, or gas6 inhibitory nucleic acid molecule. In one embodiment, the cell expresses a decreased level of a siat7e, lama4, cdkl3, cox15, egr1, or gas6 nucleic acid molecule or polypeptide relative to a control cell.

In yet another aspect, the invention provides a cell containing a mutation that alters the expression or activity of a polypeptide selected from the group consisting of cdkl3, siat7e, lama4, cox15, egr1, gas6, map3k9, and gap43. In one embodiment, the mutation is a deletion, missense mutation, or frameshift. In another embodiment, the cell is a mammalian cell in vitro. In yet another embodiment, the cell has altered growth characteristics relative to a control cell.

In various embodiments of any of the above aspects, the altered growth characteristics are increased or decreased adhesive characteristics. In still other embodiments, altered adhesive characteristics are measured by cell aggregation or in a shear flow chamber. In still other embodiments, the altered growth characteristics are increased cell density or an increased cell population size relative to a control cell. In still other embodiments, the cell expresses increased levels of a recombinant protein relative to a control cell. In still other embodiments of any of the above aspects, the agent is an expression vector that encodes the gene identified according to the method of the first aspect or a gene selected from any one or more of cdkl3, siat7e, lama4, cox15, egr1, gas6, map3k9, and gap43. In yet another embodiment, the agent is an inhibitory nucleic acid molecule (e.g., siRNA, shRNA, or antisense oligonucleotide) that reduces the expression of the gene identified according to a previous aspect in the cell. In still other embodiments, the gene is any one or more of siat7e, lama4, cdkl3, cox15, egr1, and gas6.

### DEFINITIONS

By “agent” is meant any small molecule chemical compound, antibody, nucleic acid molecule, or polypeptide, or fragments thereof.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and more preferably a 50%, 75%, 85%, 100% or greater change in expression levels.

By “anchorage-dependent cell” is meant a cell that requires interaction with a substrate for its survival, growth, or proliferation.

By “anchorage-independent cell” is meant a cell that does not require interaction with a substrate for its survival, growth, or proliferation.

By “cell growth characteristics” is meant the properties that define the growth of an unaltered reference cell. Such properties include cell aggregation, rate of cell proliferation, cell adhesion, or cell mortality.

By “cellular adhesion” is meant a cell-cell interaction or a cell-substrate interaction. Methods of measuring cell adhesion are known in the art and are described herein. In particular, such methods include measuring cell aggregation or measuring a cell-substrate interaction in a shear flow chamber.

By “cdkl3 nucleic acid molecule” is meant a nucleic acid molecule that encodes a Cdkl3 polypeptide. An exemplary cdkl3 polynucleotide is provided at GenBank Accession No. NM_{—}016508.

By “Cdkl3 polypeptide” is meant a polypeptide having substantial identity to GenBank accession No. NP_{—}057592 or a fragment thereof having kinase activity.

By “coil 5 nucleic acid molecule” is meant a nucleic acid molecule that encodes a cox15 polypeptide. An exemplary cox15 polynucleotide is provided at GenBank Accession No.: NM_{—}078470.

By a “cox15 polypeptide” is meant a polypeptide having substantial identity to GenBank Accession No. NP_{—}510870 or a fragment thereof having cytochrome oxidase activity.

By a “cdkl3 nucleic acid molecule” is meant a nucleic acid molecule that encodes a cdkl3 polypeptide. An exemplary cdkl3 nucleic acid molecule is provided at GenBank Accession No: NM016508.

By a “cdkl3 polypeptide” is meant a polypeptide having substantial identity to GenBank Acession No. NP_{—}057592 or a fragment thereof having cdkl3 kinase activity.

By “cellular mortality” is meant a cell not having the ability to continue to grow and divide indefinitely. Cells that continue to grow and divide indefinitely are “immortalized cells.”

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “differentially expressed” is meant an increase or decrease in the expression of a polynucleotide or polypeptide relative to a reference level of expression.

By “egr1 nucleic acid molecule” is meant a nucleic acid molecule encoding an egr1 polypeptide. An exemplary egr1 nucleic acid molecule is provided at GenBank Accession No. BC073983.

By “egr1 polypeptide” is meant protein having substantial identity to GenBank Accession No. NP_{—}001955 or a fragment thereof having early growth response activity.

By “gas6 nucleic acid molecule” is meant a polynucleotide encoding a gas6 polypeptide. An exemplary gas6 nucleic acid molecule is provided at GenBank Accession No. NM_{—}000820.

By “gas6 polypeptide” is meant a protein having substantial identity to GenBank Accession No. NP_{—}000811 or a fragment thereof having growth arrest specific activity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

Nucleic acid molecules useful in the methods of the invention include any nucleic acid molecule that encodes a polypeptide or inhibitory nucleic acid molecule of the invention or a fragment thereof (e.g., cdkl3, siat7e, lama4, cox15, egr1, gas6, map3k9, and gap43). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “lama4 nucleic acid molecule” is meant a polynucleotide that encodes a lamanin α4 polypeptide. One exemplary lama4 nucleic acid molecule is provided at GenBank Accession No. NM_{—}002290.

By “lamanin α4 polypeptide” is meant a protein having substantial identity to the amino acid sequence shown at GenBank Accession No. BC066552, or a fragment thereof having a biological activity associated with lamanin α4. Exemplary biological activities include promoting cell adhesion to a substrate.

By “modulates” is meant increases or decreases.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “promoter” is meant a polynucleotide sufficient to direct transcription. Exemplary promoters suitable for expressing a polynucleotide or polypeptide of the invention in a mammalian cell include, but are not limited to, the CMV, U6, and H1 promoters.

By “reference” is meant a standard or control condition. By “ribozyme” is meant an RNA that has enzymatic activity, possessing site specificity and cleavage capability for a target RNA molecule. Ribozymes can be used to decrease expression of a polypeptide. Methods for using ribozymes to decrease polypeptide expression are described, for example, by Turner et al., (Adv. Exp. Med. Biol. 465:303-318, 2000) and Norris et al., (Adv. Exp. Med. Biol. 465:293-301, 2000). By “siat7e nucleic acid molecule” is meant a polynucleotide that encodes a sialyltransferase 7E polypeptide. One exemplary nucleic acid sequence is provided at GenBank Accession No. NM_{—}030965.

By “sialyltransferase 7E polypeptide” is meant a protein having substantial identity to GenBank accession No. NP_{—}038471, or a fragment thereof having sialyltransferase activity.

By “substantially identical” is meant a polypeptide or nucleic acid molecule exhibiting at least 75% identity to a reference amino acid sequence (for example, any one of the amino acid sequences described herein) or nucleic acid sequence (for example, any one of the nucleic acid sequences described herein). Preferably, such a sequence is at least 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By“transgenic” is meant any cell which includes a DNA sequence which is inserted by artifice into a cell and becomes part of the genome of the organism which develops from that cell, or part of a heritable extra chromosomal array.

The invention provides compositions and methods for modulating cell growth and increasing the expression of a recombinant polypeptide. Other features and advantages of the invention will be apparent from the detailed description, and from the claims.

### BRIEF DESCRIPTION OF THE DRAWINGS

FIG. 1 is a graph showing the average expression ratios for two specific genes from four different microarray slides. The error bars indicate the standard deviation from the average, based on values from several different spots for each particular gene.

FIGS. 2A-2F are six micrographs of different cells lines at a magnification of 10×. FIG. 2A shows anchorage-dependent HeLa cells. FIG. 2B shows anchorage-dependent HeLa cells with siat7e over-expressed. FIG. 2C shows anchorage-dependent HeLa cells with lama4 blocked. FIG. 2D shows anchorage-independent HeLa cells. FIG. 2E shows anchorage-independent HeLa cells with siat7e blocked. FIG. 2F shows anchorage-independent HeLa cells with lama4 over-expressed.

FIGS. 3A and 3B are graphs that show the distribution of clusters 5-100 μm in anchorage independent HeLa cells with and without siRNA specific for siat7e.

FIGS. 4A and 4B are graphs that show the distribution of clusters 5-60 μm in anchorage independent I HeLa cells with and without lama4 gene insert.

FIGS. 5A and 5B are graphs showing cell dissociation as a function of shear stress. Panel (A) measures the percentage total cells that dissociated. The cells used were anchorage dependent HeLa cells as follows: Unmodified cell, cells with siat7e over-expressed (sialyltranferase+), and cells with lama4 blocked (laminin−). Panel (B) measures the percentage total cells that dissociated. The cells used were anchorage independent HeLa cells as follows: unmodified cells, cells with lama4 overexpressed (laminin+), and cells with siat7e blocked (sialyltranferase−).

FIGS. 6A-6C are photomicrographs showing HEK-293 cells that were untreated, transfected with an expression vector containing a nonsense gene insert, or transfected with an expression vector containing a cdkl3 insert.

FIGS. 7A-7C provide exemplary sequences of lama4, siate7, and cdkl3, and their encoded polypeptides.

FIGS. 8A and 8B show the expression vectors used to express the lama4, siate7, and cdkl3 nucleic acid molecules in mammalian cells.

FIG. 9 is a graph showing a growth curve for suspension and attached HeLa cells in bioreactors under batch conditions. Attached HeLa cells were grown on Cytodex 3 microcarriers.

FIG. 10 is a graph showing median expression ratios for cdkl3 and cox15 from four different spotted cDNA microarray slides. The median expression ratio is calculated from 3 or more gene-specific spots on a single slide. The error bars indicate the range in expression ratios observed for a given slide.

FIG. 11 shows Western blot analysis for several different cell lines (HeLa, HEK-293 and CHO) indicating relative expression levels of cdkl3 and cox15. The control cells (control) were transfected with blank plasmids.

FIG. 12 is six panels of graphs showing the results of flow cytometric analysis. The graphs indicate cell count vs. fluorescence. In each image the first peak (also the largest peak) indicates cells in the G0/G1 phases whereas the second, smaller peak indicates cells in the G2/M phases. In between these two peaks are cells in the S phase. The percentages of cells in each phase are shown along side each figure.

FIG. 13 shows 9 panels if images of three different HEK-293 cell types grown at varying fetal bovine serum levels (i.e. 10%, 5%, and 2%) indicating morphological changes. HEK-293 cells expressing egr1 or gas6 are indicated as are HEK-293 control cells. All of these images were taken with a DM IRB microscope and attached camera. Morphological changes can be detected in these images.

FIG. 14 is a graph showing growth curves for HEK-293 cells grown in spinner flasks at varying serum levels. Cells were grown on Cytodex 3 microcarriers.

FIG. 15 (A and B) shows that without going through an adaptation process, cells deprived of serum for extended periods of time begin to undergo apoptosis as evidenced by decreasing viability. FIG. 15A shows the results of a Western blot analysis of HEK-293

cells transfected with: 1—egr1, 2—control, 3—gas6, 4—control. (numbers correspond to lanes). Cells were grown at 10% FBS. FIG. 15B is a graph showing cell viability vs. time after serum withdrawal.

FIG. 16 is a graph that shows cell viability vs. serum level (% by volume). During the adaptation process, the cell population displayed decreasing viability due to an increase in the number of dying cells.

FIG. 17 is a graph that shows the time needed to reach confluence as a function of serum level. Cultures determined to be confluent contained cells that covered in excess of 95% of the culture surface area of a 75 cm² tissue culture flask. Each flask assayed was seeded with 5×10⁴ cells.

FIG. 18 is a graph that shows flow cytometric analysis of HEK-293 cells expressing egr1 and HEK-293 control cells grown at 2% serum. Samples were taken from the middle of the exponential growth phase. Peaks were positioned to identify differences in cell cycle progression between the two cell lines. Actual data were overlapping.

FIG. 19 is a graph that shows median expression levels for gene candidates: egr1, gas6, map3k9, and gap43 at various serum levels based on normalized microarray results. The error bars indicate the range in expression levels observed for given condition (i.e. serum level).

### DETAILED DESCRIPTION OF THE INVENTION

The invention generally provides methods and compositions for altering cell properties and facilitating recombinant protein production. The invention is based, at least in part, on the observations that cell adhesive characteristics and recombinant protein production can be altered by modulating the expression of genes (e.g., cdkl3, siat7e, lama4, cox15, egr1, gas6, map3k9, and gap43) that are differentially expressed in anchorage-dependent and anchorage-independent cell lines. Specifically, recombinant polypeptide expression is increased in cells transfected with an expression vector that encodes cdkl3, cox15, egr1 or gas6; and alterations in laminin α4, sialyltransferase 7E, cdkl3, cox15, egr1 or gas6 modulate cellular adhesion.

### Cellular Adhesion

An important cellular property in biotechnology applications is adherence, which refers to a cell's ability to attach to a surface and grow. Anchorage-independent cell lines are cell lines that grow without adhering to a surface, while anchorage-dependent cell lines must adhere to a surface to grow. Depending on the biotechnology application, anchorage-independent or anchorage-dependent cell lines may be preferred. Being able to manipulate the cellular feature of adhesion would, therefore, benefit biotechnology applications.

A variety of studies have been conducted to evaluate the importance of cellular properties for the production of specific products. Researchers have also identified possible pathways to modify cellular properties by employing specific selection methods. In relation to adhesion, most studies have focused on either quantifying observations relating to adhesion at a genetic level or exploring the effects of specific compounds on adhesion. For instance, selenite, a hydrous calcium sulfate, has been shown to reduce the ability of HeLa cells to attach to fibronectin. In another series of experiments, researchers showed that blocking the expression of pten, a tumor suppressor gene, in 293T cells using siRNA resulted in a loss of adhesion as well as a change in cell morphology (Mise-Omata et al., Biochem. Biophys. Res. Commun. 328, 1034-1042). Other studies have highlighted a number of genes thought to be involved in mediating adhesion such as rhoA, rac1, and cdc42 (Mise-Omata et al., Biochem. Biophys. Res. Commun. 328, 1034-1042; Hatzimanikatis and Lee, Metab. Eng. 1, 275-281, 1999). The present invention employs bioinformatic methods to identify genes that are differentially expressed in anchorage-dependent vs. anchorage independent cells. In addition, the method provides methods for modulating the adhesive characteristics of cells.

### Recombinant Polypeptide Expression

The invention provides methods for enhancing the production of recombinant proteins or recombinant polynucleotides encoding such proteins. In some embodiments, the enhancement in production of a protein (e.g., a recombinant protein) is achieved by modulating the expression of a polypeptide of the invention (e.g., cdkl3, siat7e, lama4, cox15, egr1, gash, map3k9, and gap43) and altering the growth properties (e.g., adhesion, proliferation) properties of the cell. Specifically, the methods increase the expression of polypeptides, including therapeutic biologicals, such as antibodies, cytokines, growth factors, enzymes, immunomodulators, thrombolytics, glycosylated proteins, secreted proteins, and DNA sequences encoding such polypeptides. Recombinant polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle.

Construction of transgenes can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used for the disclosed constructs. One skilled in the art will appreciate that a promoter is chosen that directs expression of the chosen gene in a cell of interest. Any promoter that regulates expression of a nucleic acid sequence described herein can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is advantageous, however, that an intact region of the transcriptional regulatory elements of a gene is used. Once a suitable transgene construct has been made, any suitable technique for introducing this construct into cells can be used.

Those skilled in the field of molecular biology will understand that any of a wide variety of expression systems may be used to provide the recombinant protein. The precise host cell used is not critical to the invention. A polypeptide of the invention is preferably expressed in a prokaryotic or eukaryotic host cell (e.g., Saccharomyces cerevisiae, insect cells, e.g., Sf21 cells, or mammalian cells, e.g., HEK-293, NIH 3T3, HeLa, CHO, COS, MDCK cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Polypeptides and Analogs

Also included in the invention are recombinant polypeptides or fragments thereof that are modified in ways that enhance or inhibit their ability to be expressed by a cell of the invention. The invention provides methods for optimizing an amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Analogs have a chemical structure designed to mimic the reference proteins functional activity. Such analogs are administered according to methods of the invention. Protein analogs may exceed the physiological activity of the original polypeptide. Methods of analog design are well known in the art, and synthesis of analogs can be carried out according to such methods by modifying the chemical structures such that the resultant analogs increase the reprogramming or regenerative activity of a reference polypeptide. These chemical modifications include, but are not limited to, substituting alternative R groups and varying the degree of saturation at specific carbon atoms of a reference fusion polypeptide. Preferably, the fusion protein analogs are relatively resistant to in vivo degradation, resulting in a more prolonged therapeutic effect upon administration. Assays for measuring functional activity include, but are not limited to, those described in the Examples below.

### Inhibitory Nucleic Acids

Inhibitory nucleic acid molecules are nucleobase oligomers that inhibit the expression of a cdkl3, siat7e, lama4, cox15, earl, gash, map3k9, or gap43 nucleic acid molecule or polypeptide. Such oligonucleotides can be used to generate cells having altered growth characteristics (e.g., altered cell-cell or cell-substrate adhesion, rate of proliferation, growth to particular cell density) that are desirable for certain applications. Such oligonucleotides include single and double stranded nucleic acid molecules (e.g., DNA, RNA, and analogs thereof) that bind a nucleic acid molecule that encodes a siat7e, lama4, cdkl3, cox15, egr1 or gas6 polypeptide (e.g., antisense molecules, siRNA, shRNA) as well as nucleic acid molecules that bind directly to a siat7e, lama4, cdkl3, cox15, egr1 or gas6polypeptide to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of siat7e, lama4, cdkl3, cox15, egr1 or gas6 gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of siat7e, lama4, cdkl3, cox15, egr1 or gas6expression. In one embodiment, siat7e, lama4, cdkl3, cox15, egr1 or gas6expression is reduced in a CHO or HEK cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505, 2002, each of which is hereby incorporated by reference. RNA Polymerase III promoters suitable for the expression of an siRNA in a mammalian cell include the well-characterized U6 and H1 promoters. U6 and H1 promoters are used to drive the expression of siRNAs in mammalian cells (Sui et al., Proc Natl Acad Sci USA 99, 5515-5520, 2002, Brummelkamp et al Science 296:550-553, 2002).

Antisense Oligonucleotides

Inhibitory nucleic acid molecules include antisense oligonucleotides that specifically hybridize with one or more siat7e, lama4, cdkl3, cox15, egr1 or gas6 polynucleotides. The specific hybridization of the nucleobase oligomer with siat7e, lama4, cdkl3, cox15, egr1 or gas6 polynucleotide (e.g., RNA, DNA) interferes with the normal function of that siat7e, lama4, cdkl3, cox15, egr1 or gas6 polynucleotide, reducing the amount of siat7e, lama4, cdkl3, cox15, egr1 or gas6polypeptide produced.

The invention features a nucleobase oligomer of up to about 30 nucleobases in length. Desirably, when administered to a cell, the oligomer inhibits expression of siat7e, lama4, cdkl3, coil 5, egr1 or gas6. A nucleobase oligomer of the invention may also contain, e.g., an additional 20, 40, 60, 85, 120, or more consecutive nucleobases that are complementary to an siat7e, lama4, cdkl3, cox15, egr1 or gas6 polynucleotide. The nucleobase oligomer (or a portion thereof) may contain a modified backbone. Phosphorothioate, phosphorodithioate, and other modified backbones are known in the art. The nucleobase oligomer may also contain one or more non-natural linkages.

Ribozymes

Catalytic RNA molecules or ribozymes that include an antisense siat7e, lama4, cdkl3, cox15, egr1 or gas6 sequence of the present invention can be used to inhibit expression of a siat7e, lama4, cdkl3, cox15, egr1 or gas6 nucleic acid molecule. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591, 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 base pair (desirably 25 to 29 bp), and the loops can range from 4 to 30 by (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Knockdown of Polypeptide Expression

As described in more detail below, cells having reduced expression of siat7e, lama4, cdkl3, cox15, egr1 or gash have altered growth characteristics (e.g., altered cell-cell or cell-substrate adhesion, rate of proliferation, growth to particular cell density) that are desirable for certain applications. Such cells are generated using any method known in the art. In one embodiment, a targeting vector is used that creates a knockout mutation in a gene of interest. The targeting vector is introduced into a suitable cell line to generate one or more cell lines that carry a knockout mutation. By a “knockout mutation” is meant an artificially-induced alteration in a nucleic acid molecule (created by recombinant DNA technology or deliberate exposure to a mutagen) that reduces the biological activity of the polypeptide normally encoded therefrom by at least about 50%, 75%, 80%, 90%, 95%, or more relative to the unmutated gene. The mutation can be, without limitation, an insertion, deletion, frameshift mutation, or a missense mutation. The targeting construct may result in the disruption of the gene of interest, e.g., by insertion of a heterologous sequence containing stop codons, or the construct may be used to replace the wild-type gene with a mutant form of the same gene, e.g. a “knock-in.” In another example, FRT sequences may be introduced into the cell such that they flank the gene of interest. Transient or continuous expression of the FLP protein is then used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789, 1996).

Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66, 2000)).

Conditional knockout cells are also produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends in Genetics 9:413-421, 1993).

### Recombinant Polypeptide Expression

Methods for expressing a recombinant polypeptide, such as a therapeutic biological, involve the transfection of cells of the invention (e.g., cells having altered siat7e, lama4, cdkl3, cox15, egr1 or gash) with a nucleic acid molecule encoding a recombinant protein, variant, or fragment thereof. Such nucleic acid molecules can be delivered to cells in vitro or to the cells of a subject having a disease or disorder amenable to treatment with the recombinant polypeptide. The nucleic acid molecules must be delivered to the cells in a form in which they can be taken up so that therapeutically effective levels of the protein or a fragment thereof can be produced.

Transducing viral (e.g., retroviral, adenoviral, and adeno-associated viral) vectors can be used for polynucleotide expression, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a therapeutic protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al.; N. Engl. J. Med 323:370, 1990; Anderson et al., U.S. Pat. No. 5,399,346). For some applications, a viral vector is used to administer a polynucleotide.

Non-viral approaches can also be employed for the introduction of therapeutic to a cell where recombinant protein expression is desired. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression of a recombinant protein can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### EXAMPLES

It should be appreciated that the invention should not be construed to be limited to the examples that are now described; rather, the invention should be construed to include any and all applications provided herein and all equivalent variations within the skill of the ordinary artisan. As described in more detail below, the experiments reported herein identified genes influencing cellular growth with the intent of engineering cell lines by manipulating the expression of these genes.

This strategy of applying bio-informatics techniques to characterize and manipulate cell phenotype is a powerful tool for altering the properties of various cell lines to achieve desired biotechnology objectives. As reported herein, variations in gene expression were identified using cDNA and oligonucleotide microarrays between HeLa cells having anchorage-dependent and anchorage-independent growth requirements. Based on these studies, siat7e and lama4 genes, among others, were selected for further study. siat7e encodes a type II membrane glycosylating sialyltransferase. The expression ratios for these two genes in anchorage-dependent and anchorage-independent HeLa cells was confirmed using RT-PCR. Once verified, the expressions of these genes was manipulated in vivo, and the adhesive characteristics of the altered cells was characterized. An adhesion assay was developed to quantify the effects of altering gene expression levels. This assay involved the use of a shear flow chamber to assay a cell's adhesive characteristics. Decreasing the expression of siat7e using siRNA in anchorage-independent HeLa cells increased the relative number of multi-cellular aggregates. Increasing the expression of lama4 in anchorage-independent HeLa cells also increased the relative number of multi-cellular aggregates. By altering the expression of either siat7e or lama4 it was possible to-detect differences in adhesion properties.

### Example 1

### siat7e and lama4 were Differentially Expressed in Anchorage-Dependent and Anchorage-Independent HeLa Cells

The growth of anchorage-dependent and anchorage-independent HeLa cells was carried out in bio-reactors under similar conditions. Samples for microarray analysis were taken at time points when both cell lines had cell populations at roughly the same confluence (i.e., slightly above 90%) and approximately the same metabolic characteristics as measured by glucose concentration (3-4 g/L), lactate concentration (0.5-1 g/L), and pH (6.8-7.4).

Total RNA samples from anchorage-dependent and anchorage-independent HeLa cell cultures were purified and used to construct cDNA, which was analyzed using cDNA and oligonucleotide microarrays. Initial data analysis was carried out using the image analysis software for microarrays program from GENEPIX PRO, from Molecular Devices Corporation (Sunnyvale, Calif.) to visualize the hybridization on each of four hybridized cDNA microarray slides. Over 8,400 genes were found to be of sufficient quality to be used to collectively normalize the data using both total intensity and regression models. Following normalization, the data was filtered by categorizing the quality of the spots constituting a gene's expression ratio. An initial round of screening identified approximately 3,500 differentially expressed genes.

The identified genes had intensity values greater than the local background intensity as determined during normalization. A subsequent round of screening reduced this number to 667 genes. The primary criterion used in screening was the determination that at least three expression ratios for a particular gene be within 25% of the median expression ratio. From this screening, two genes were selected for further analysis. The gene siat7e was expressed at a higher level in anchorage-independent HeLa cells than in anchorage-dependent HeLa cells, and lama4 was expressed at a lower level in anchorage-independent HeLa cells than in anchorage-dependent HeLa cells. siat7e had a relatively high median expression ratio, and lama 4 had a relatively low median expression ratio. These data are shown in Table 1, below.

TABLE 1
Gene Function

| Gene | |
|---|---|
| Symbol | Functionality of Gene Products |
| siat7e | Involved in glycosylation |
| | Exhibits higher activity with glycolipids |
| | than with glycoproteins |
| | Type II membrane protein (Golgi) |
| lama4 | Complex glycoprotein that mediates the adhesion, |
| | migration and organization of cells into tissues |
| | during embryonic development by interacting with |
| | other extra cellular matrix components. Member |
| | of family of extra cellular matrix glycoproteins. |

### Example 2

### siate7e and lama4 are Differentially Expressed In Anchorage-Dependent Vs. Anchorage-Independent HeLa Cells

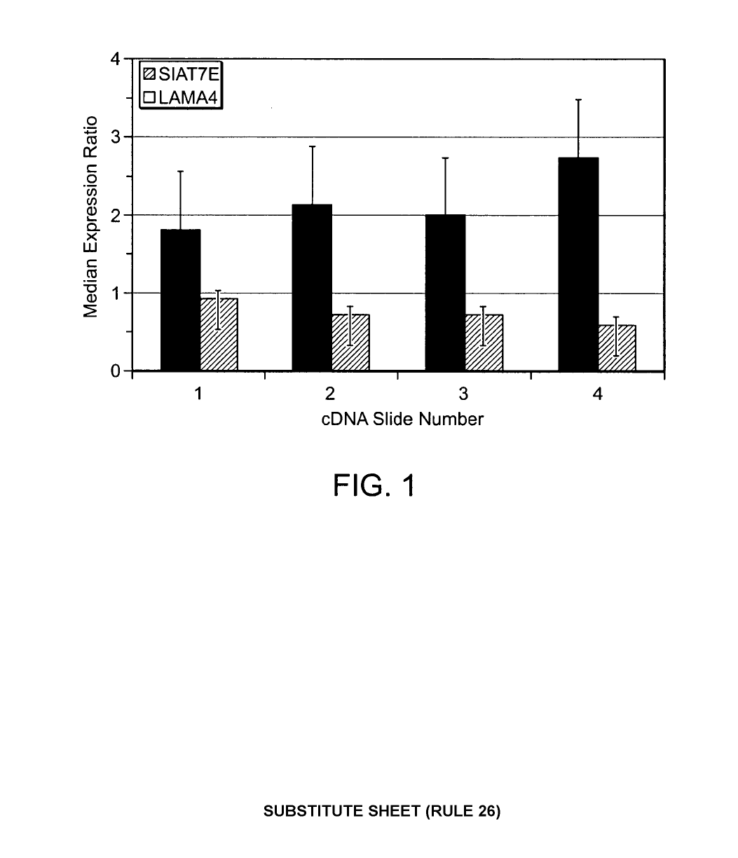

Expression ratios for these two genes are illustrated in FIG. 1 for each of the four hybridized cDNA arrays. Regardless of the assay method, the expression ratios of siat7e were consistently above 1, while the expression ratios for lama4 were consistently below 1. The inherent variability between cDNA slides in terms of expression ratios for the two selected genes established a need for verification of the results. To validate the results from the cDNA arrays, two pairs of oligonuceotide arrays as well as a series of RT-PCR experiments was conducted. The results of these experiments is shown below in Table 2, which demonstrates that the median expression levels across all three platforms are in line with one another for both genes.

TABLE 2
Median expression ratios for 2 specific genes by different platforms;
cDNA microarrays, oligonucleotide microarrays, and RT-PCR

| Gene | | | Oligonu- | RT- |
|---|---|---|---|---|
| symbol | Gene title | cDNA | cleotide | PCR |
| siat7e | Alpha-N-aceytlgalactosaminide | 2.061 | 2.350 | 2.21 |
| | Alpha-2 6 sialytranferase V | | | |
| lama4 | Laminin alpha-4 chain precursor | 0.734 | 0.667 | 0.76 |

### Example 3

### Increased siat7e Expression Decreased Anchorage-Dependent Growth and Altered Cell Morphology

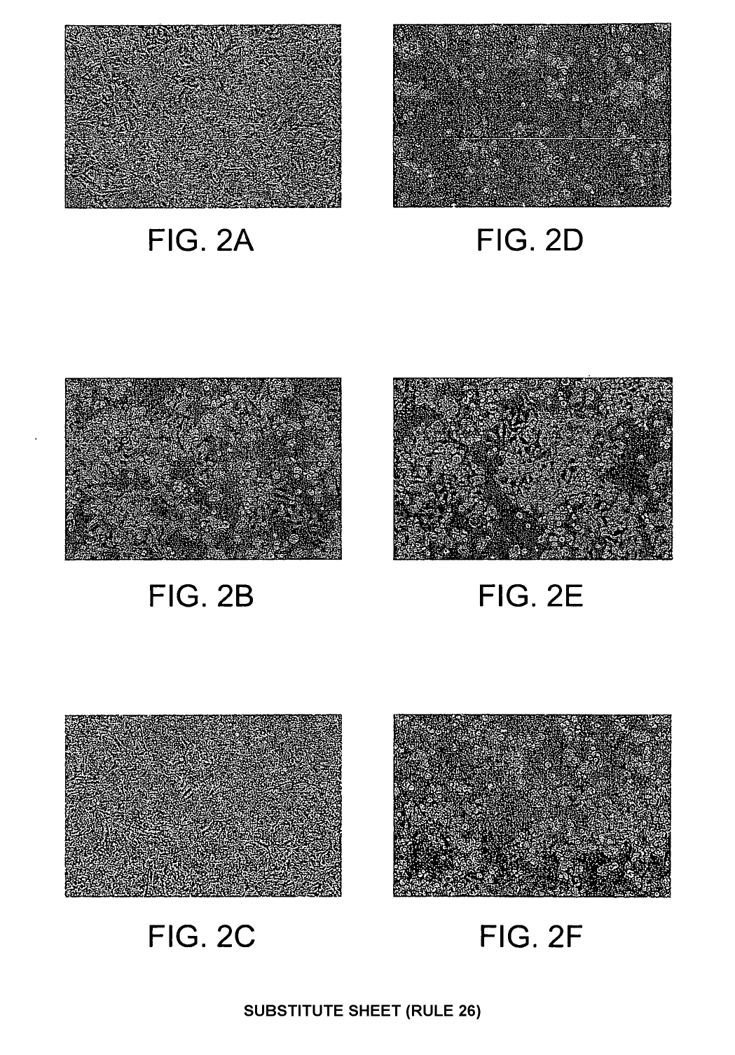

Transcription of siat7e was lower in anchorage-dependent HeLa cells than in anchorage-independent HeLa cells, whereas transcription of lama4 was lower in anchorage-independent HeLa cells than in anchorage-dependent HeLa cells. If these genes function in the adhesion and/or growth characteristics of HeLa cells, then enhancing or inhibiting their expression should affect cell physiology. To evaluate the effect of siat7e on cell adhesion, the expression of siat7e in anchorage-dependent HeLa cells was increased by transfecting the cells with the full-length gene. The images of anchorage-dependent HeLa cells shown in panel FIG. 2A were compared with those of corresponding HeLa cells over-expressing siat7e shown in FIG. 2B. Enhancing the expression of siat7e decreased the percentage of viable cells attached to the culture surface and changed cell shape. The change in cell shape observed in some cells was a change from elongated, cylindrical cells to compact, spherical cells. In addition, these cells grew to higher cell densities when cultured for the same period of time under the same growth conditions (i.e. same media, temperature).

### Example 4

### Decreased Lama4 Expression Decreased Anchorage-Dependent Growth and Altered Cell Morphology

Inhibiting the transcription of lama4 in anchorage-dependent HeLa cells by transfecting the cells with gene-specific siRNA also resulted in a morphological change as illustrated in FIGS. 2A and 2C. The image of anchorage-dependent HeLa cells, shown in FIG. 2A was compared with the image of anchorage-dependent HeLa cells expressing an siRNA that targeted lama4 shown in FIG. 2C. This comparison shows that there is a clear change in cell morphology in cells expressing that lama4 gene-specific siRNA. In addition, there was a decrease in the percentage of cells attached to the culture surface. These results parallel those as observed when e the expression of siat7e was enhanced in anchorage-dependent HeLa cells. Simultaneously altering expression levels of siat7e and lama4 in anchorage-dependent HeLa cells did not enhance the observed changes in cell morphology or anchorage-dependent cell growth.

### Example 5

### Cell Adhesiveness Increased in Cells Expressing a siat7e siRNA

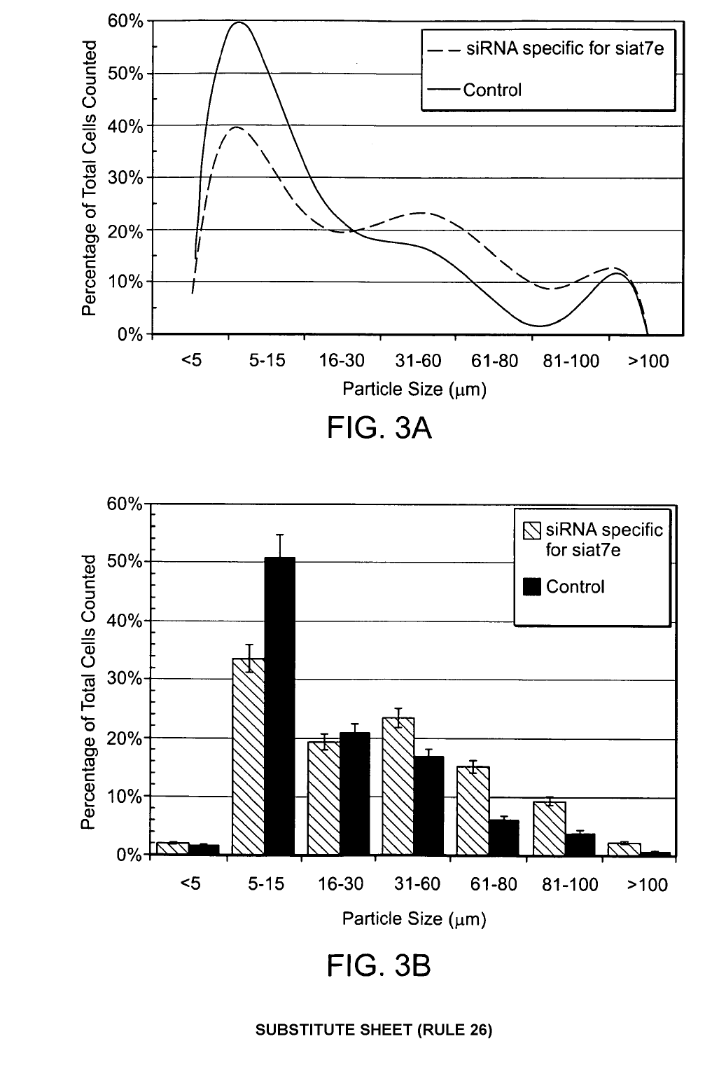

The impact of inhibiting the expression level of siat7e and increasing the expression of lama4 in anchorage-independent HeLa cells was also examined. The effect of blocking the expression of siat7e using gene-specific siRNA is shown in FIGS. 2 and 3. The image of anchorage-independent HeLa cells shown in FIG. 2D was compared to the image of anchorage-independent HeLa cells expressing an siRNA that targets siat7e (FIG. 2E) to determine the effect of inhibiting the expression of siat7e on anchorage-independent HeLa cells. Inhibition of siat7e resulted in an increase in the percentage of cells adhering to the surface, an increase in the percentage of cells adhering to one another (i.e. clumping), and a decrease in the percentage of cells detached from the surface. In addition, a greater percentage of cells appeared to elongate and take on shapes that were non-spherical, especially when attached to the culture surface. To further characterize the growth of anchorage independent HeLa cells following down regulation of siat7e, an assay was devised to measure size distribution of cells in culture.

As seen in the control populations in FIGS. 3A and 3B, one-half of anchorage-independent HeLa cells existed individually (5-15 μm) because an individual HeLa cell is roughly 10 μm in diameter at the narrowest portion of the cell. For siat7e-specific siRNA treated cells, the percentage of individually existing cells was reduced to one-third. In addition, there was an increase in the percentage of cells existing in clusters of 3-6 cells that form an aggregate having a diameter of about 60 μm. Differences between siat7e-specific siRNA treated cells and control cells was statistically significant for three ranges, 5-15 μm, 61-80 μm, and 81-100 μm using a one-tailed Student's t-test with at least a 10% significance level. Indeed, the anchorage independent (siat7e-) cells exhibited greater clustering at all three size groupings above 60 μm.

### Example 6

### Increased lama4 Expression Resulted in Increased Cell Adhesiveness

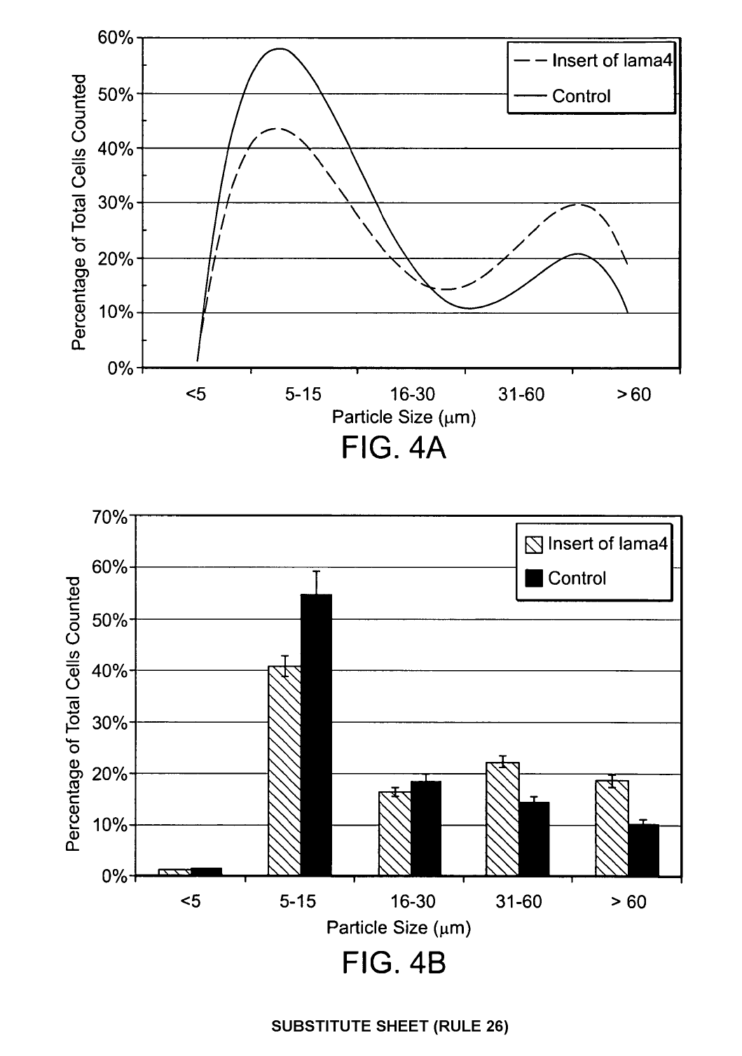

The results of enhancing the expression of lama4 in anchorage-independent HeLa are shown in FIGS. 2A-2E and 4A and 4B. The image of anchorage-independent HeLa cells shown in FIG. 2D, was compared with the image of anchorage-independent HeLa cells over-expressing lama4 shown in FIG. 2F. This comparison indicated that over-expressing lama4 in anchorage-independent HeLa cells resulted in an increase in cell adherence as measured by the percentage of cells adhering to the surface or clumping together; as well as resulting in an altered cell morphology. Cells having increased lama4 expression changed from a spherical to an elongated shape. FIGS. 4A and 4B show the results of a cell size distribution comparison between anchorage-independent HeLa cells over expressing lama4 and corresponding control cells. Enhancing the expression of lama4 in anchorage-independent HeLa cells resulted in a reduction in the percentage of cells existing individually (55% to 41%), and an increase in the percentage of cells existing in clusters; a phenomenon similar to that observed when the expression of siat7e was blocked in anchorage-independent HeLa cells. Differences between anchorage-independent HeLa cells with inserts of lama4 and control cells were statistically significant for two ranges; 5-15 μm and >60 μm, as determined using a one-tailed Student's t-test with at least a 10% significance level. The clusters greater than 60 μm were not separated into individual categories since there were fewer large clusters in the anchorage independent HeLa over expressing lama4 compared with anchorage independent HeLa with blocked siat7e. The probability associated with these differences occurring naturally was sufficiently small to make it unlikely that these differences would occur on their own.

### Example 7

### Quantifying Adhesion Properties in Cells with Modified Gene Expression

To further characterize the adhesive properties of cells having altered levels of lama4 or siat7e expression, stable cell lines were constructed. Two of the cell lines constructed constitutively expressed the siat7e or lama4 genes, and two of the cell lines expressed an siRNA that targeted the siat7e or lama4 genes. The adhesive properties of these cell lines was characterized using a shear flow chamber.

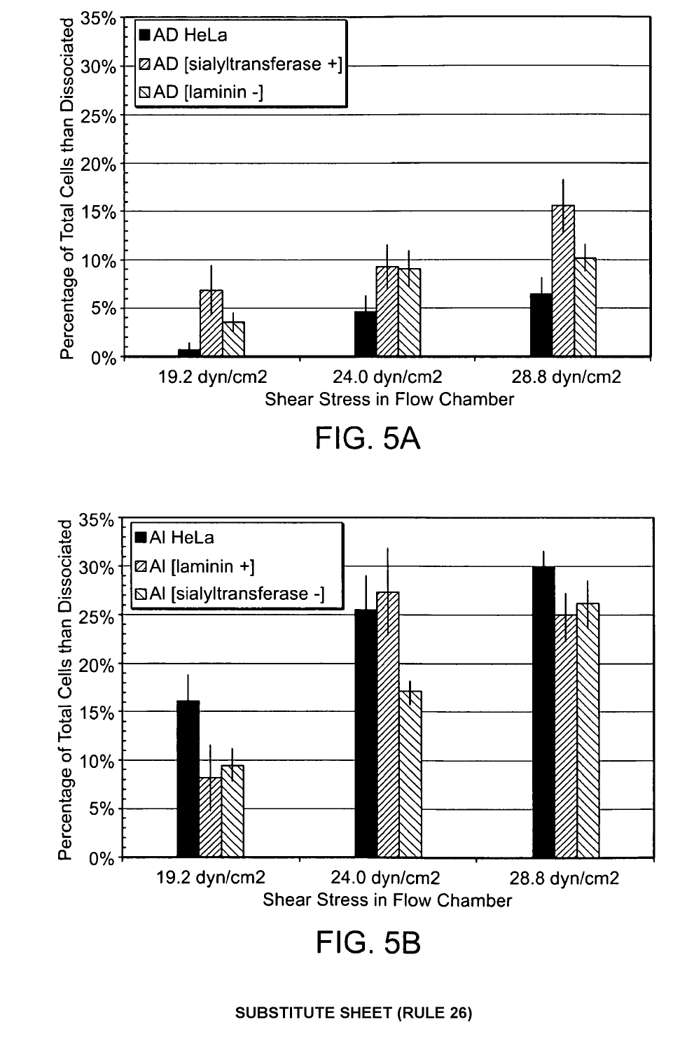

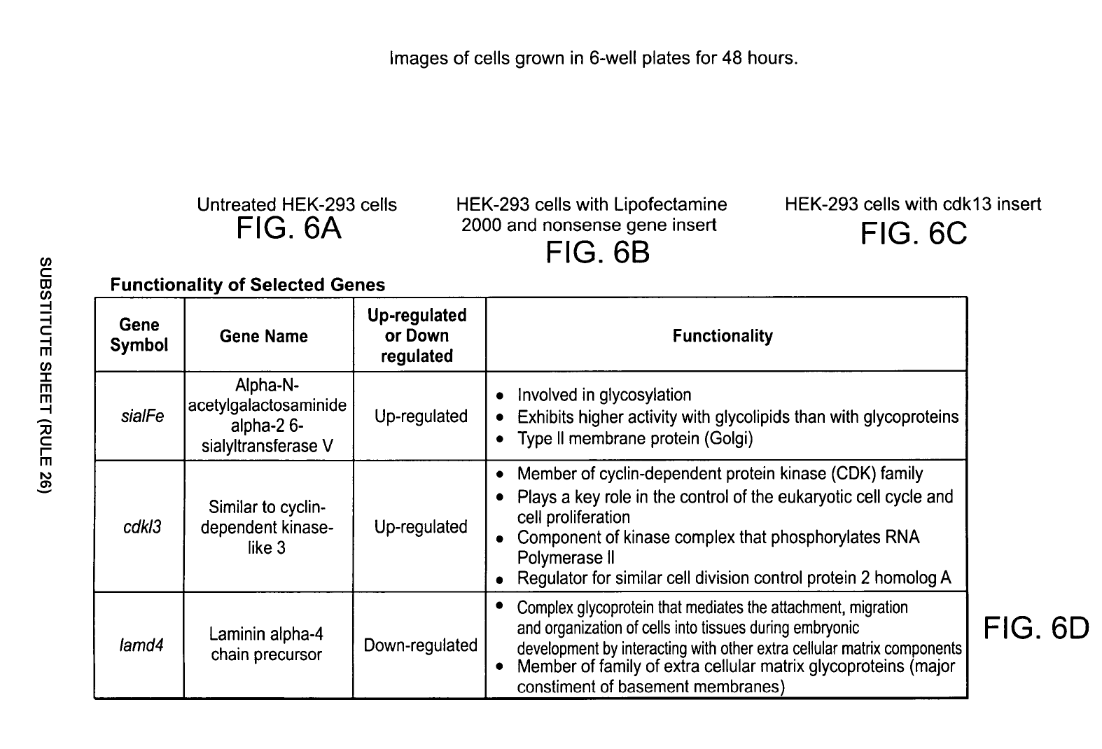

First, the adhesive properties of anchorage-dependent HeLa and anchorage-independent HeLa cells was characterized to determine a range of shear stresses that distinguished between the two cell lines having different growth characteristics. Shear stresses in these ranges were then used to study the adhesive characteristics of cells constitutively expressing siat7e or lama4 genes or expressing an siRNA that targeted the siat7e or lama4 genes. A summary of the percentage of cells dissociated for the unmodified anchorage-dependent HeLa cells, anchorage-dependent HeLa cells with enhanced siat7e expression and anchorage-dependent HeLa cells with reduced lama4 expression at different shear stresses is shown in FIG. 5A. The rate of dissociation and the percentage of cell dissociation with increasing shear stress were greater for both modified anchorage-dependent cell lines than for the unmodified anchorage-dependent cells, which likely indicates weakened adhesion. The percentage dissociation for the unmodified anchorage independent HeLa cells, anchorage-independent HeLa cells with reduced siat7e expression and anchorage independent HeLa cells with enhanced lama4 expression at different shear stress are shown in FIG. 5B. The percentage of cells dissociated was lower for the anchorage-independent cells with reduced siat7e expression compared to the unmodified anchorage-independent HeLa cells and anchorage-independent cells with increasing lama4 expression. In general all the anchorage independent cells were less adherent than the anchorage dependent cell lines. By averaging multiple runs for each cell line and converting the number of dissociated cells into percentages it was possible to identify differences in adhesion between the different cell lines. When the expression of lama4 was blocked in anchorage-dependent HeLa cells using siRNA, the percentage of cells dissociating increased from 12%±3% to 23%±4%. When the expression of siat7e was blocked in anchorage-independent HeLa cells, the percentage of cells dissociating decreased from an average of 72%±4% to 53%±4%. Over-expression of these genes also modified adhesion ability was observed for the reciprocal cases; independently.

Together the data identify two genes that play a role in the adhesion of HeLa cells, and show the effect of these genes on anchorage dependent and independent cell-growth characteristics.

### Example 8

### Cells containing a cdkl3 expression vector express increased levels of recombinant proteins

In related studies, anchorage-independent HeLa cells were found to grow substantially faster than anchorage-dependent HeLa cells. Specifically, anchorage-independent HeLa cells reached higher cell densities in a given amount of time than anchorage-dependent HeLa cells cultured under the same conditions. Gene expression analysis using cDNA microarrays identified cdkl3 as a gene that is differentially expressed in anchorage-dependent HeLa cells relative to anchorage-independent HeLa cells. Cdkl3 is a member of the cyclin kinase family. To test the hypothesis that increased cdkl3 expression causes an increase in cell proliferation, HEK 293 cells were transiently transfected with an expression vector containing cdkl3. HEK-293 cells are an adherent human embryonic kidney epithelial cell line.

The growth characteristics of HEK-293 cells over-expressing cdkl3 were then compared to the growth characteristics of control HEK-293 cells. Both sets of cells were grown under controlled conditions in bio-reactors with monitored and controlled pH, CO₂ concentration, and oxygen levels, and temperature. Surprisingly, cells containing the cdkl3 expression vector reached a higher cell density and a higher population size in a given amount of time relative to corresponding control cells that were not transfected with the cdkl3 expression vector. The cdkl3-expressing cells also showed a 2.5-fold increase in the expression of a recombinant protein, protein adipocyte complement-related protein of 30 kDa (ACRP30) as quantitated using an ELISA assay. Similar results were obtained when Chinese Hamster Ovary cells were transiently transfected with cdkl3.

### Example 9

### Enhancement of Cell Proliferation and Protein Production in Various Mammalian Cell Lines by Gene Insertion of a Cyclin-Dependent Kinase Homolog

In further experiments on modifying cellular growth characteristics, cyclin-dependent kinase like 3 (cdkl3) and cytochrome c oxidase subunit (cox15) were found to be up-regulated in faster growing, anchorage-independent (suspension) HeLa cells relative to slower growing, anchorage-dependent (attached) HeLa cells. Enhanced expression of either gene in the attached HeLa cells resulted in elevated cell proliferation, though insertion of cdkl3 had a greater impact than that of cox15. Moreover, flow cytometric analysis indicated that cells with an insert of cdkl3 were able to transition from the G0/G1 phases to the S phase faster than control cells. In turn, expression of cox15 was seen to increase the maximum viable cell numbers achieved relative to the control, and to a greater extent than cdkl3. Quantitatively similar results were obtained with two Human Embryonic Kidney-293 (HEK-293) cell lines and a Chinese Hamster Ovary (CHO) cell line. Additionally, HEK-293 cells secreting adipocyte complement-related protein of 30 kDa (acrp30) exhibited a slight increase in specific protein production and higher total protein production in response to the insertion of either cdkl3 or cox15. The effect of cdkl3 on cell growth is consistent with its homology to the cdk3 gene which is involved in G1 to S phase transition. Likewise, the increase in cell viability due to cox15 expression is consistent with its role in oxidative phosphorylation as an assembly factor for cytochrome c oxidase and its involvement removing apoptosis-inducing oxygen radicals. The use of microarray technology to identify genes influential to specific cellular processes raises the possibility of engineering cell lines as desired to meet production needs.

In addition, as described in Example 10, a Human Embryonic Kidney-293 (HEK-293) cell line initially propagated in 10% fetal bovine serum (FBS) was gradually adapted to SFM and analyzed at specific serum levels using oligonucleotide microarrays. A number of genes were found to be differentially expressed several of which were then verified using RT-PCR. Two genes, egr1 and gas6, were selected for further analysis based on their level of differential expression, overall expression patterns, and proposed functionalities. HEK-293 cells propagated in 10% FBS were transfected with either egr1 or gas6 and then adapted to SFM. Another HEK-293 cell line constitutively secreting a protein, acrp30 (adipocyte complement-related protein of 30 kDa), was also transfected with either egr1 or gas6 and then adapted to SFM. Results indicated higher expression of either egr1 or gas6 enhanced the ability of both cell lines to adapt to SFM by maintaining higher viability levels and improved growth rates at low serum levels. Egr1 appeared to have a greater impact on adaptability than gas6. In order to ascertain the effects, if any, egr1 had on protein production an ELISA for acrp30 was conducted. Results illustrated specific protein production was unaltered when the expression of egr1 was increased. In addition, flow cytometric experiments revealed increased expression of egr1 was closely correlated with an increase in the percentage of cells in the G0 phase.

### Growth of HeLa Cells in Bioreactors

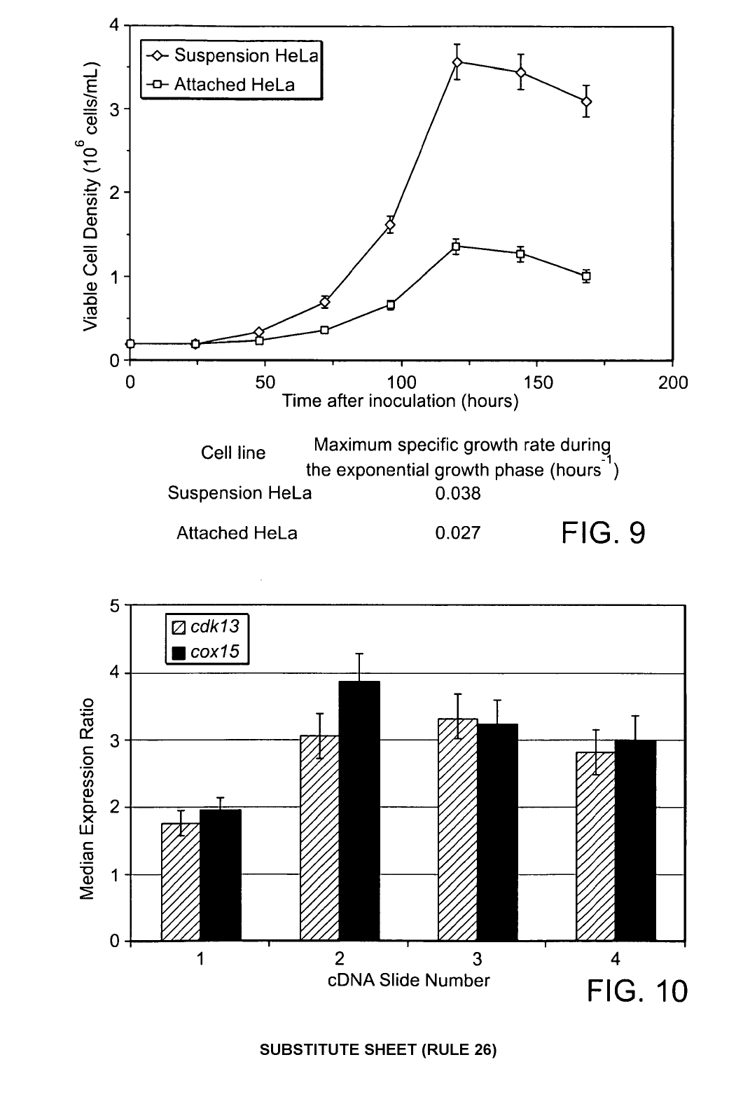

Both the attached and suspension HeLa cell lines were grown concurrently in bioreactors in three independent experiments using the same media and culture environment. However, the attached HeLa cells had to be grown using microcarriers. The measured viable cell densities for both cells lines are shown in FIG. 9 along with the corresponding growth curves from typical runs. The suspension HeLa cells grew at a maximum specific growth rate of 0.038±0.002 h⁻¹, more than 40% higher than the attached HeLa cells which grew at a maximum specific growth rate of 0.027±0.001 h⁻¹. The suspension HeLa cells also achieved a maximum cell density of about 3.5×10⁶ cells/mL, more than twice the maximum cell density obtained for the attached HeLa cells. The viability for each cell line was above 90% until the stationary phase of growth was reached.

### Comparison of Gene Expression Levels Between Suspension and Attached Hela Cells

Specimens for microarray analysis were taken at the same time from each bioreactor, at regular intervals corresponding to different regions of growth. Imaging software was used to determine which comparisons exhibited the most significant differences between the two cell lines and had the highest levels of overall transcriptional activity. Samples from the middle of the exponential growth phase corresponding to the maximum specific growth rates were found to meet these criteria. This information was deciphered by examining scanned images of hybridized microarrays using GenePix, a visualization software program.

Analysis of the hybridized cDNA microarrays began with quality control (i.e. removing spots of poor signal) prior to implementing total intensity normalization (Burke, Mol Diagn 2000, 5:349-357; Quackenbush, Nat Rev Genet. 2001, 2:418-427). Subsequently, the data were filtered by removing genes lacking consistency between slides in terms of how close the expression ratio from any one slide was to the median expression ratio calculated from all of the slides. The data were also screened for genes with expression ratios from the slides in the same direction (i.e. either upregulated or downregulated across all of the slides).

Using clustering algorithms, the genes were further organized into groups (Quackenbush, Nat Rev Genet. 2001, 2:418-427). Principle component analysis (PCA) and gap statistic were used to estimate the number and size of groups inherently present in the data. Results of these algorithms indicated groups of 8, 9, 13, or 14 were likely to form. With this information, self-organizing maps (SOMs) and hierarchical clustering were then applied to the data to segregate the genes into distinct groups. These clusters were probed to identify subsets of genes with relevance to cellular growth based on known or proposed functionalities related to cell cycle regulation, apoptosis, and/or signal transduction. Genes that could be categorized in this manner were then interrogated further based on the level of differential expression with a sampling of the results shown in Table 3.

TABLE 3
Gene names and maximum/minimum expression ratios
of the form: suspension HeLa/attached HeLa.

| | | Maximum | Minimum |
|---|---|---|---|
| Gene | | Expression | Expression |
| Symbol | Gene Name | Ratio | Ratio |
| cdkl3 | cyclin-dependent kinase-like 3 | 3.32 | 1.76 |
| cox15 | cytochrome c oxidase assembly | 3.87 | 1.95 |
| | protein | | |
| agpat2 | 1-acylglycerol-3-phosphate | 3.13 | 1.64 |
| | O-acyltransferase 2 | | |
| fgf7 | Fibroblast growth factor 7 | 3.50 | 1.75 |
| dtymk | deoxythymidylate kinase | 4.05 | 1.92 |
| | (thymidylate kinase) | | |
| arhf | ras homolog gene family f | 2.95 | 1.70 |
| plagl1 | pleiomorphic adenoma gene-like 1 | 0.04 | 0.02 |
| tial1 | cytotoxic granule-associated RNA | 0.68 | 0.14 |
| | binding protein-like 1 | | |

Of these genes, cdkl3 [GenBank: NM016508] and cox15 [GenBank: NM078470] were selected for further analysis. As shown in the graph in FIG. 10, both cdkl3 and cox15 had expression levels greater than 1, indicating higher expression in the suspension HeLa cell line than in the attached HeLa cell line.

### Verification of Microarray Results

As can be seen in Table 3 and FIG. 10, the expression ratios of both cdkl3 and cox15 varied over the different samples. A series of RT-PCR experiments were conducted, which confirmed these results as shown in Table 4. Two controls were used: gapd and pgk1.

TABLE 4
Median expression ratios of the form: suspension
HeLa/attached HeLa from microarrays and RT-PCR.

| Gene | | | |
|---|---|---|---|
| Symbol | Gene Name | cDNA | RT-PCR |
| cdkl3 | cyclin-dependent kinase- | 2.74 | 2.6 ± 0.2 |
| | like 3 | | |
| cox15 | cytochrome c oxidase | 3.01 | 3.4 ± 0.3 |
| | assembly protein | | |
| gapd | Glyceraldehyde-3- | 1.03 | 1.2 ± 0.1 |
| | phosphate dehydrogenase | | |
| pgk1 | Phosphoglycerate kinase 1 | 0.98 | 1.0 ± 0.1 |

The findings in the Table demonstrate the expression levels are consistent between the microarray data and the RT-PCR experiments.
Enhanced Expression of cdkl3 and cox15 in Attached HeLa Cells

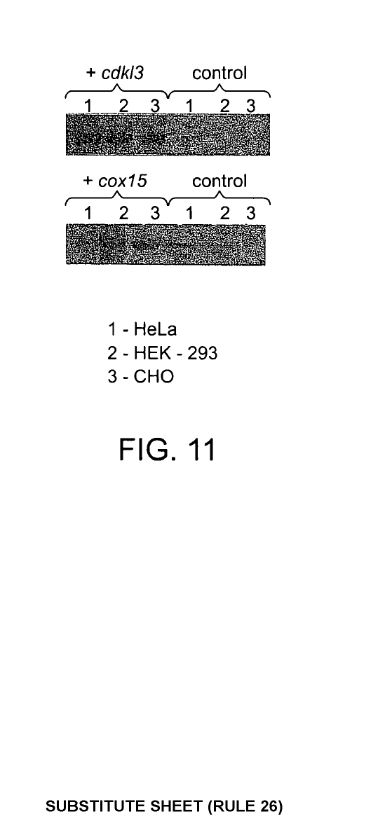

To evaluate the impact of these two genes in vivo, DNA plasmids containing either cdkl3 or cox15 were transfected into the attached HeLa cells. Cells transfected with only blank plasmids (i.e. plasmids containing neither cdkl3 nor cox15) served as the control. Each plasmid also contained the neomycin gene, which allowed cells to be selected based on resistance to geneticin. A number of clones were selected in this manner and then assayed for expression of the translated protein using western blots, shown in FIG. 11. These results indicate that cells transfected with either cdkl3 or cox15 had significantly higher protein levels than the control cells.

The growth profiles/characteristics of the attached HeLa cells transfected with cdkl3, cox15, or a blank plasmid are summarized in Table 5, shown below.

TABLE 5
Growth-related data for varying cell lines grown in spinner flasks

| | | Maximum growth rate during the exponential | | | | |
|---|---|---|---|---|---|---|
| | | growth phase (hours⁻¹) from 3 different runs | | | | |
| | | | | | Average | Maximum viable |
| | | | | | Change vs. | cell density |
| Cell Type | Cell line | Run 1 | Run 2 | Run 3 | Control (%) | (10⁶ cell/mL) |
| HeLa | control | 0.027 | 0.026 | 0.026 | | 1.49 ± 0.03 |
| | + cdkl3 | 0.031 | 0.032 | 0.032 | 20 | 1.53 ± 0.03 |
| | + cox15 | 0.030 | 0.030 | 0.031 | 15 | 1.66 ± 0.02 |
| HEK-293 | control | 0.030 | 0.030 | 0.032 | | 1.98 ± 0.02 |
| | + cdkl3 | 0.035 | 0.037 | 0.037 | 19 | 2.04 ± 0.04 |
| | + cox15 | 0.034 | 0.036 | 0.034 | 13 | 2.15 ± 0.05 |
| HEK-293 | control | 0.031 | 0.031 | 0.032 | | 1.94 ± 0.04 |
| ACRP30 | + cdkl3 | 0.037 | 0.037 | 0.035 | 16 | 2.01 ± 0.02 |
| | + cox15 | 0.036 | 0.035 | 0.036 | 14 | 2.10 ± 0.06 |
| CHO | control | 0.033 | 0.034 | 0.034 | | 1.95 ± 0.01 |
| | + cdkl3 | 0.036 | 0.037 | 0.037 | 9 | 2.04 ± 0.02 |
| | + cox15 | 0.036 | 0.035 | 0.035 | 5 | 2.09 ± 0.03 |
| MDCK | control | 0.029 | 0.030 | 0.030 | | 1.12 ± 0.04 |
| | + cdkl3 | 0.030 | 0.029 | 0.030 | 0 | 1.10 ± 0.02 |
| | + cox15 | 0.028 | 0.029 | 0.028 | −5 | 1.08 ± 0.07 |

All three cell lines were grown on microcarriers in 250 mL spinner flasks. The maximum specific growth rates, determined in three independent experiments, revealed that the attached HeLa cells transfected with a plasmid containing either cdkl3 or cox15 grew faster than the control cells. Cells expressing cdkl3 had a maximum specific growth rate that was 20% greater than the control cells, while the cells expressing cox15 had a maximum specific growth rate that was 15% greater than the control cells. Using a t-test, it was determined that the differences in maximum specific growth rates between treated (i.e. cells with plasmids containing either cdkl3 or cox15) and control cells were statistically significant for α-values (also referred to as risk level) as low as 0.005. The α-value is the chance associated with finding a statistically significant difference between means when there is none. For instance, a value of 0.005 corresponds to 5 instances out of 1,000 in which the difference could be by chance. It was also observed that the duration of the lag phase in cells expressing either cdkl3 or cox15 was 5-8% shorter than that of the control cells.

In addition, cells expressing cox15 were able to achieve a maximum viable cell density 11% higher than the control cells whereas cells expressing cdkl3 achieved a maximum viable cell density equivalent to the control cells (shown in Table 5). The difference between cells expressing cox15 and control cells was statistically significant for α-values as low as 0.005. Similar results, both in terms of growth rates and cell densities, were obtained when these cell lines were grown in T-flasks. Furthermore, neither gene impacted cell viability nor were any additive effects observed when both genes were simultaneously expressed.

### Gene Sequence Homology and Enhanced Expression of Cdkl3 and Cox15 in Other Cell Lines

Using two online databases, Harvester and GenBank, the nucleotide sequences for both cdkl3 and cox15 were determined. Sequence analysis was performed to quantify homology between varying species. For the cdkl3 gene, the following were determined: 94% similarity with Canis familiaris (dog), 83% similarity with Rattus norvegicus (rodent), and 90% similarity with Mus musculus (mouse). Slightly less similar was the cox15 gene with 87% similarity to Canis familiaris (dog), 86% similarity to Rattus norvegicus (rodent), and 86% similarity to Mus musculus (mouse). Based on these results, three additional cell lines, HEK-293, CHO, and MDCK were chosen for investigation. The HEK-293 cells were selected to investigate whether or not the genes identified using HeLa cells had functionality in other human-derived cell lines. With significant homology between the three rodent species in terms of gene transcripts, CHO cells were also chosen due to their widespread use in commercial applications. However, without a central database detailing the sequence of the CHO genome, the existence of homologs for either cdkl3 or cox15 could not be verified in that species. The canine cell line selected, MDCK, is commonly used in the production of vaccines.

When grown under the same spinner flask conditions previously described, two different types of HEK-293 cells (HEK-293 and HEK-293 ACRP30) grew faster than the control cells (i.e. cells transfected with the blank plasmid) when expressing either cdkl3 or cox15 (Table 5). The maximum specific growth rate of the HEK-293 cells expressing cdkl3 was 19% greater than the control cells; a value statistically significant for α-values as low 0.005. Similarly, the maximum specific growth rate of the HEK-293 cells expressing cox15 was 13% greater than the control cells; a value statistically significant for α-values as low 0.01. In addition, cells expressing cox15 grew to a maximum viable cell density nearly 9% higher than the control cells, statistically significant for α-values as low 0.005.

HEK-293 ACRP30 cells expressing cdkl3 had a maximum specific growth rate 16% higher than the control cells (as shown in Table 5). Using a t-test, this value was deemed significant for α-values as low 0.005. HEK-293 ACRP30 cells expressing cox15 also had a maximum specific growth rate higher than the control cells, by approximately 14%. This value was determined to be statistically significant for α-values as low 0.001. Additionally, the HEK-293 ACRP30 cells achieved a maximum viable cell density more than 8% greater than the control cells; statistically significant for α-values as low 0.01.

CHO cells with inserts of either cdkl3 or cox15 were also found to grow faster than control cells, as shown in Table 5. CHO cells expressing cdkl3 had a maximum specific growth rate 9% higher than the control cells; a value statistically significant for α-values as low 0.005. CHO cells expressing cox15 had a maximum specific growth rate 5% higher than the control cells; statistically significant for α-values as low 0.025. In addition, cells expressing either cdkl3 or cox15 achieved maximum viable cell densities 5% (α-value≧0.005) or 7% (α-value≧0.001) greater than the control cells, respectively. In contrast, MDCK cells with inserts of either cdkl3 or cox15 did not exhibit differences in terms of growth rates or maximum viable cell densities, as shown in Table 5.

### Examining Cell Proliferation Using Flow Cytometry and ELISA

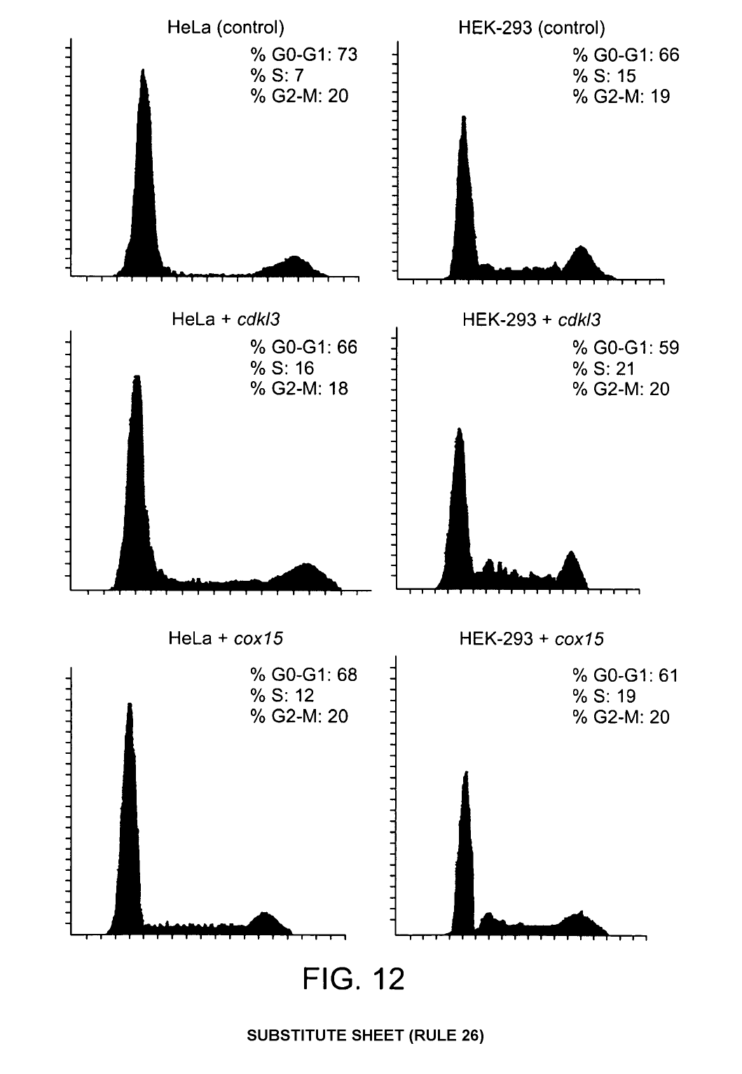

In order to evaluate the effects of enhanced expression of either cdkl3 or cox15 on cell cycle, analysis of cell cycle progression was performed using flow cytometry. The cell line exhibiting the greatest effects of enhanced expression of either cdkl3 or cox15 was the attached HeLa cell line. In this assay, cells were initially synchronized and then grown up until late into the exponential phase. Cells with enhanced expression of either cdkl3 or cox15, shown in FIG. 12, exhibited a lower percentage of cells in the G0-G1 phases and a higher percentage of cells in the S phase when compared to control cells. The percentage of cells in the G2-M phases was similar between the three cell lines.

To investigate the potential effects of enhanced expression of either cdkl3 or cox15 on protein production HEK-293 cells constitutively secreting recombinant adipocyte complement-related protein of 30 kDa (acrp30) (HEK-293 ACRP30) were transfected with plasmids containing either cox15, cdkl3, or the blank plasmid. Cells were plated in T-flasks at an equivalent seeding density and allowed to grow to confluency. The total protein production levels in ng/mL were measured in each flask along with the productivities of acrp30 on a per cell basis. The results are shown in Table 6.

TABLE 6
ELISA results for a typical run of the HEK-293 ACRP30 cells

| Cell line | Viable cell density (10⁶ cells/mL) | Total protein production (ng) | $\begin{matrix}Average specific \\ protein production \\ ( \frac{ng \cdot mL}{cells} )\end{matrix}$ |
|---|---|---|---|
| control with | 1.24 ± 0.01 | 1580 ± 130 | 1.27 × 10⁻³ |
| plasmid | | | |
| + cdkl3 | 1.40 ± 0.02 | 1900 ± 160 | 1.36 × 10⁻³ |
| + coxl5 | 1.31 ± 0.03 | 1750 ± 190 | 1.34 × 10⁻³ |

The total protein levels were increased by 20% in the cells transfected with cdkl3 while an 11% increase was seen in cells transfected with cox15, relative to the control cells. Interestingly, productivities were also higher for the cells expressing these two genes.

Using clustering algorithms driven by gene ontology, differentially regulated genes with functionalities relevant to cell growth were identified when comparing attached HeLa cells to suspension HeLa cells'(Table 3). Two of the genes selected for further investigation, cdkl3 and cox15, were expressed at higher levels in the suspension than in the attached HeLa cells. However, each gene was thought to relate to cellular growth in a distinct way. Previous studies suggest cdkl3 (cyclin-dependent kinase like 3) is involved in cell cycle regulation/progression, while cox15 (cytochrome c oxidase assembly protein) is involved in energy metabolism (Yee et al., Biochem Biophys Res Commun 2003, 308:784-79). The present study supports these and other proposed functions for both cdkl3 and cox15 as elaborated below.

The gene cdkl3 encodes a polypeptide of 455 amino acids titled NKIAMRE [Swiss-Prot: Q8IVW4] that localizes in the cytoplasm and is thought to be a component of a kinase complex that phosphorylates the C-terminus of RNA polymerase II (Yee et al., Biochem Biophys Res Commun 2003, 308:784-792). Although, the precise functionality of NKIAMRE remains unknown, sequence similarities and the presence of several motifs together imply membership to both the mitogen-activated protein kinase (mapk) family and cyclin-dependent kinase (cdk) family. Both of these families are integral to a variety of cellular processes including signaling, cell cycle regulation, migration, and survival.

As the gene name suggests, cdkl3 is most similar in sequence to cyclin-dependent kinase 3 (cdk3); a gene that encodes a kinase believed to be necessary for the G1-S transition in mammalian cells. The protein encoded by cdkl3 also contains the highly conserved TXY (Threonine-X-Tyrosine) motif found in the activation loop domains of either a mapk or cdk (Yee Biochem Biophys Res Commun 2003, 308:784-792; Hanks et al., Science 1988, 241:42-52). In addition, two residues, a serine and a tyrosine, in subdomain I closely resemble an adenosine triphosphate (ATP) binding domain and a negative regulation site typical of a cdk. The protein's name itself, NKIAMRE signifies an amino acid sequence thought to be a cyclin-binding domain also found in a cdk (Yee Biochem Biophys Res Commun 2003, 308:784-792). Additionally, the catalytic domain of NICIAMRE contains two highly conserved sequences found in both serine/threonine and tyrosine protein kinases (Yee Biochem Biophys Res Commun 2003, 308:784-792; Hanks et al., Science 1988, 241:42-52). More recently, high expression of nkiamre was detected in two aggressive types of anaplastic large cell lymphoma (ALCL) tumors when compared to peripheral blood lymphocytes (Thompson et al., Hum Pathol 2005, 36:494-504). This finding suggests the gene may also play a role in pathogenesis and/or tumorigenesis.

Collectively, cdkl3 appears to act as either a cell cycle mimic or regulator with several distinct functions including ATP & nucleotide binding, kinase activity, and transferase activity; all of which relate back to regulation of the cell cycle and control of cellular proliferation. The current study supports these claims and builds upon the notion that cdkl3, like its homolog cdk3, influences the cell cycle through observations of increased growth rates, reduced lag phases, and greater protein production. Indeed, cdk3 is thought to be rate-limiting for mammalian cell cycle progression. Additional studies have shown mammalian cells require cdk3 to exit the G0 and G1 phases as well as enter the S phase. These observations are also supported by the present work through flow cytometry experiments that indicated cells with an insert of cdkl3 were able to transition from the G0/G1 phases to the S phase faster than cells without the insert.

Conversely, the human gene cox15 could relate to cellular growth from the perspective of energy metabolism (i.e. cells growing faster have greater energy demands). It encodes a cytochrome c oxidase (COX) assembly protein referred to as COX15 [Swiss-Prot: Q7KZN9] which localizes in the mitochondrial inner membrane. Significant expression of cox15 has been observed in tissues with high oxidative phosphorylation demands such as muscle, heart, and brain (Oquendo et al., J Med Genet. 2004, 41:540-54432).

Acting as the terminal enzyme of the respiratory chain, COX is responsible for the transfer of electrons to molecular oxygen, contributing to the generation of ATP via the proton motive force. COX15 is one of several accessory proteins not part of the overall complex, but relevant to the structure, synthesis, and processing of COX. Because COX is a large and intricate complex that is continually being used, assembly proteins are also thought to regulate its activity. It should also be noted, that COX is thought to be one of the rate-controlling steps of oxidative phosphorylation. Without wishing to be bound by theory, these ideas suggest that higher expression of cox15 could improve the efficiency of respiration. Support for this can be found in the present study in that cells with an insert of cox15 grew faster and produced more recombinant protein (acrp30) than cells without the insert.

Previous studies have also shown COX15 is involved in the synthesis of heme a, a compound with greater binding affinity than other heme molecules and believed to be critical to energy conservation during oxygen reduction by COX. In fact, defects in the cox15 gene have resulted in heme a deficiencies whereas defects in COX function have lead to the onset of degenerative diseases such as Leigh syndrome and encephalohepatopathy (Oquendo et al., J Med Genet. 2004, 41:540-544). To date, only a few cases of these diseases have been the result of a cox15 mutation; and in those select cases the mutations were heterogeneous suggesting the loss of functional COX15 may be fatal. Additionally, it has been proposed that compounds like heme a when bound to proteins, can facilitate the removal of damaging and apoptosis-triggering oxygen radicals. Without wishing to be bound by theory, these observations suggest that higher cox15 expression might counteract apoptosis, bolstering cell viability. Indeed, this notion is consistent with findings of the present study in that cells with inserts of cox15 achieved higher viable cell densities than cells without the insert.

The results presented herein have illustrated the value of comparing two similar cell lines in a microarray format and the subsequent use of bioinformatics tools to uncover natural cellular factors that may contribute to enhanced cell growth and improved survival in cell culture environment. The suspension HeLa cells tended to grow much faster than the attached HeLa cells and it was this physiological difference that was exploited to elucidate some of the potential genetic reasons for this difference. Although a number of candidates were identified, the present work demonstrated that two genes are particularly relevant to enhanced growth and increased viabilities, cdkl3 and cox15. Furthermore, the proposed functions for these genes, obtained from previous studies, are consistent with the changes that were observed when over-expressed in several mammalian cell lines. The fact that the changes were not universal may be indicative of specific differences in controlling the cell cycle and/or the relative role and endogenous activities of these genes in particular organisms. Understanding how to alter or enhance cell growth will be useful in understanding potential genes associated with cancer as indeed cdkl3 has been found to be upregulated in particular tumors. Likewise, the critical anti-apoptosis role of COX15 may explain the reason only heterogeneous mutations in the gene have been observed in diseases and why other diseases in COX function lead to degenerative disorders such as Leigh Syndrome and encephalohepatopathy. In addition, the ability to control the cell cycle and apoptosis can be useful in cell engineering for biotechnology applications by providing greater cell mass and improved yields of recombinant protein.

### Example 10

### Egr1 and gas6 Expedite the Adaptation of HEK-293 Cells to Serum-Free Media by Conferring Survivability

As described herein, oligonucleotide microarrays were also used to evaluate the gene transcriptional levels of HEK-293 (Human Embryonic Kidney) cells as they were gradually adapted to media devoid of serum. Data analysis detected a number of genes with expression patterns correlated, directly or indirectly, to decreasing serum levels. The influence of several of these genes including egr1 and gas6, were investigated by transfecting a single gene into two different HEK-293 cell lines. The transfected cell lines were monitored for growth, viability, and ability to adapt to serum free media. Flow cytometry and ELISA experiments were also conducted to document changes in cell cycle progression during the adaptation process as well as protein production, respectively.

### Growth of HEK-293 Under Serum Deprivation Conditions

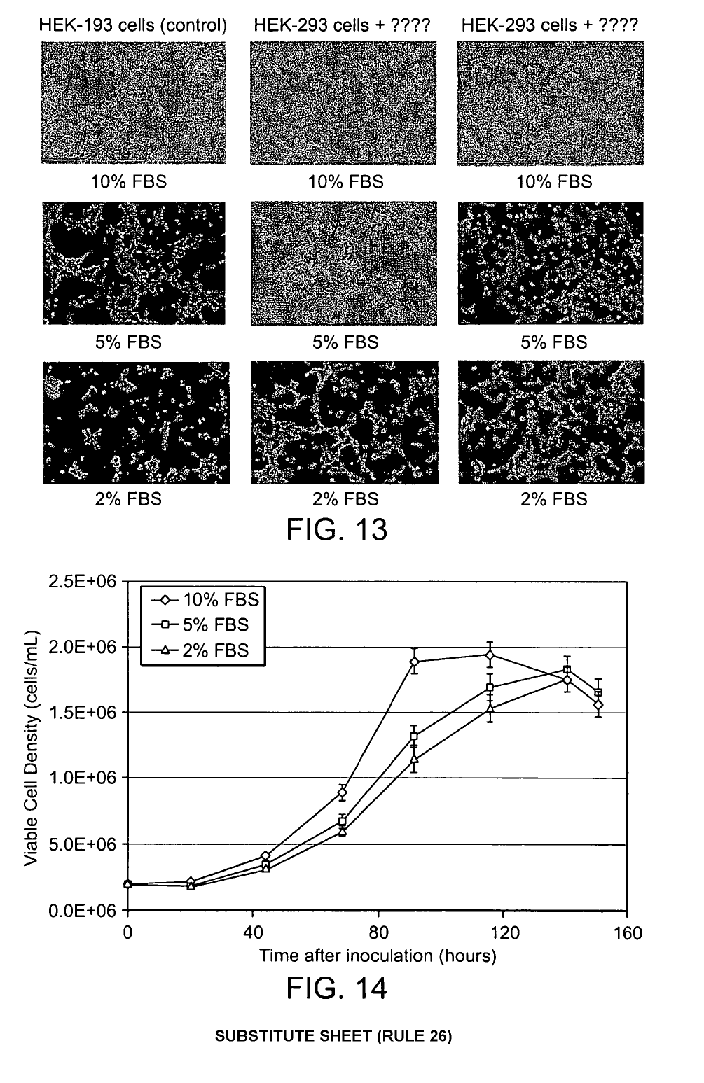

HEK-293 cells were initiated from frozen vials and propagated in 10% FBS in tissue-culture flasks. These cells were also grown in spinner flasks with microcarriers. Gradually, the cells were adapted to grow in serum-free media (SFM) by continual passage at lower serum levels, also referred to as sequential adaptation. During the adaptation process, it was observed that the cells grown at low serum levels (i.e. serum levels≦2% by volume) exhibited both morphological changes and a loss of adhesion, as shown in FIG. 13. At low serum levels, viable cells became more spherical and compact in shape while also detaching from the surface and aggregating with other cells. In addition, at low serum levels, cells displayed altered growth kinetics as compared to cells grown at higher serum levels, as shown in FIG. 14. When compared to cells propagated at high serum levels, cells propagated at low serum levels remained in the lag phase for longer periods of time, exhibited reduced viabilities, and displayed lower specific growth rates. Table 7, below, shows the maximum specific growth rates for 2 different HEK-293 cell lines grown at progressively lower serum levels.

TABLE 7

| | Maximum specific growth rate during the | | |
|---|---|---|---|
| | exponential growth phase (hours⁻¹) | | |
| Cell line | 10% FBS | 5% FBS | 2% FBS |
| HEK 293 | 0 032 ± 0.02 | 0 029 ± 0.02 | 0.028 ± 0.01 |
| HEK-293 ACRP30 | 0.031 ± 0.01 | 0.029 ± 0.03 | 0.026 ± 0.03 |

### Comparison of Gene Expression Levels and Evaluation of Candidates

Using oligonucleotide microarrays, HEK-293 cells grown at varying levels of serum were analyzed. Following normalization and background removal using Partek Pro software, Acuity software was used to probe the data for genes with expression patterns closely related to decreasing serum levels. Clusters of genes with expression patterns consistently increasing or decreasing at reducing serum concentrations were then identified. Genes with such expression patterns were thought to be particularly relevant to adaptation. The results of this method were explored based on gene ontology; searching for genes related to any of the following categories: cellular growth, anti-apoptosis, and cell cycle regulation. The results are presented in Table 8, below, which identifies several genes considered candidates for further investigation along with average expression levels per gene. The expression levels shown in Table 8 are normalized intensity values and are specific for cells grown at specific serum levels.

TABLE 8
Genes identified as having expression patterns correlating with
serum content, as determined by application of Acuity software.

| | Median Expression Level | | | |
|---|---|---|---|---|
| | 10% | 5% | 2% | |
| Gene Name | FBS | FBS | FBS | SFM |
| Early growth response 1 (egr1) | 627 | 885 | 1890 | 6410 |
| Growth arrest specific 6 (gas6) | 122 | 939 | 2710 | 10900 |
| Mitogen-activated protein | 216 | 366 | 4250 | 5110 |
| kinase 9 (map3k9) | | | | |
| Interleukin 6 receptor (il6r) | 132 | 464 | 1290 | 3320 |
| Mitochondrial translational | 819 | 1790 | 4170 | 5570 |
| release factor 1 like (mtrf1l) | | | | |
| histidine triad nucleotide | 2460 | 1110 | 736 | 196 |
| binding protein 3 (hint3) | | | | |
| Annexin A1 (anxa1) | 721 | 2770 | 6310 | 9740 |
| Serine/threonine/tryrosine | 1270 | 823 | 261 | 147 |
| interacting protein (styx) | | | | |
| Mortality factor 4 like 2 (morf4l2) | 389 | 1480 | 2240 | 5180 |
| Growth associated protein 43 (gap43) | 2010 | 1530 | 877 | 205 |

All of these genes were evaluated by altering their expression levels individually in vivo with the use of plasmids and/or siRNA. Transfected cells were assayed for growth and viability under varying conditions.

Genes with expression patterns that tended to increase at each progressive decrease in serum level were thought to be particularly relevant to adaptation. Four genes, egr1, gas6, map3k9, and gap43, were explored in subsequent cell engineering studies. While the average expression level remained relatively constant, the expression levels of egr1, gas6, and map3k9 increased steadily as the serum level decreased to 0%. Alternatively, gap43 showed a progressive decrease in expression at decreasing serum levels. PCR studies were performed to confirm that the expression levels of these genes were indeed altered following reduction of serum to 5% and 2% FBS (data not shown).

Enhanced Expression of egr1 and gas6

The genes egr1 and gas6 were found to have the most impact on cell survivability and adaptation to serum-free media. Both genes were found to be up-regulated in cells grown at lower serum levels relative to cells supplemented with 10% FBS. Microarray data for these genes were verified using RT-PCR.

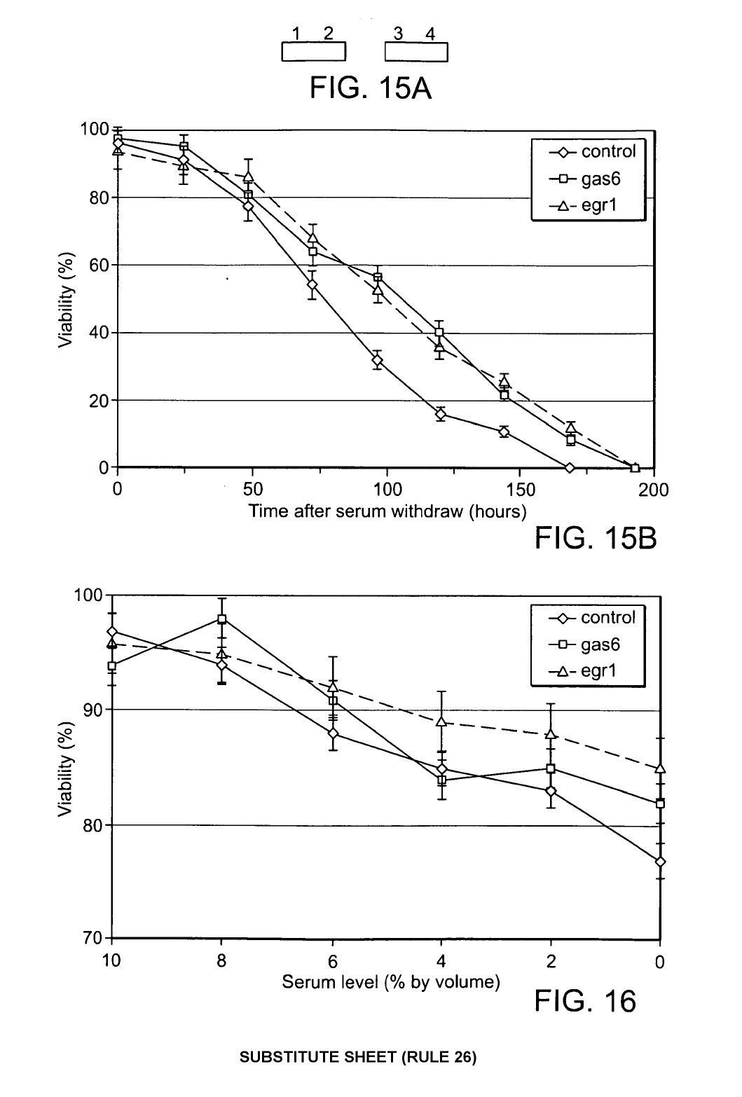

To evaluate the role these genes played in adaptation, HEK-293 cells were transfected with plasmids containing each of the four genes. Transfected cells were assayed for growth and viability during the adaptation process. HEK-293 cells transfected with either map3k9 or gap43 had no effect on adaptation whereas cells transfected with either egr1 or gas6 did exhibit altered growth characteristics. Elevated protein expression for EGR1 and GAS6 were verified using western blots, shown in FIG. 15A. Cells transfected with only blank plasmids (i.e. containing only the selection marker) served as the control in all experiments.

FIGS. 15A and 15B shows that cells transfected with either egr1 or gas6 maintained higher viabilities than control cells throughout the serum withdrawl process. In fact, after 24 hours, cells with enhanced expression of either gene had viabilities at least 10% greater than the control cells. Additionally, death (i.e. viability≈0%) in the cell population was delayed in cells transfected with either egr1 or gas6. The difference amounted to 8% (gas6) and 11% (egr1).

As shown in FIG. 16, the HEK-293 cells exhibit reduced average viabilities at low serum levels during the adaptation process. Cells expressing either egr1 or gas6 appeared to maintain higher viabilities, particularly at low serum levels. The expression of gas6 had less of an effect but the viability of HEK-293 cells transfected with gas6 was higher than the control cells at 4 out of 5 reduced serum levels.

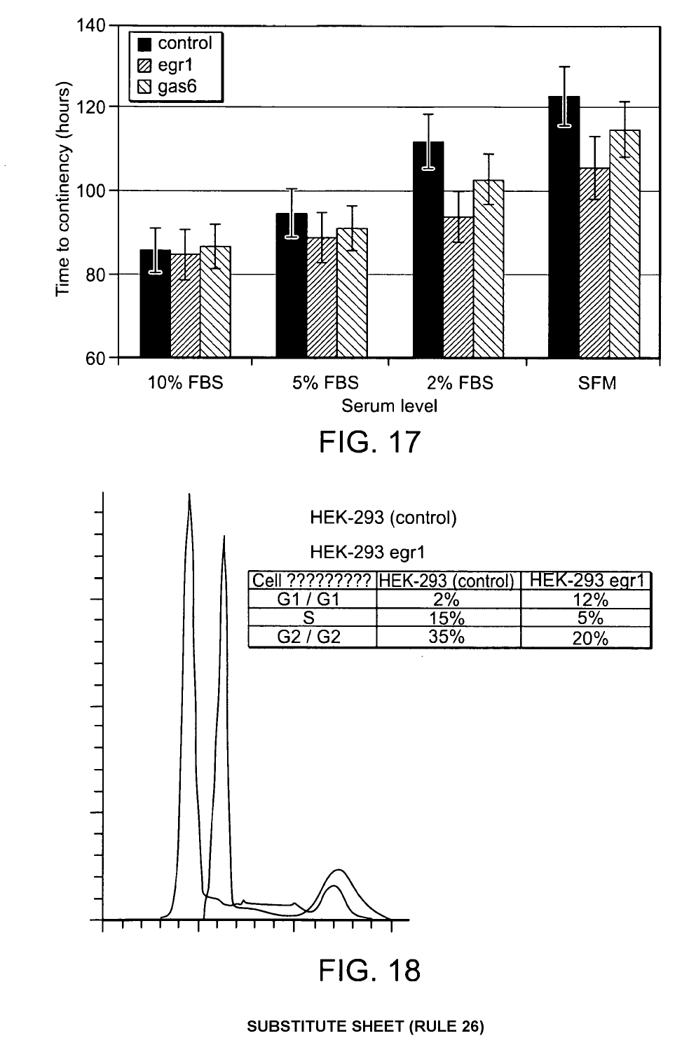

As another measure of the adaptation characteristics of the engineered cells, the time it took for cells to reach confluency was measured following seeding of flasks at a constant density in different serum environments. The ability of cells to grow to confluency faster was consistent with improved adaptation characteristics. As illustrated in FIG. 17, each of the three cell lines took progressively longer to reach confluency as the serum level was reduced. The increase in time to confluency at lower serum levels was reduced significantly in the cells engineered to express egr1 and to a lesser extent in the cells engineered to express gas6. The HEK-293 cells transfected with egr1 reached confluence (95% coverage) 15% and 12% faster than the control cells at 2% and 0% serum levels, respectively. As with the viability changes, the greatest difference related to the expression of egr1 and gas6 were seen at the lowest serum levels.

EGR1 localizes in the nucleus and belongs to a family of zinc-finger proteins involved in transcriptional regulation (Fahmy et al., Nat Med 9(8):1026-1032, 2003). EGR1 itself targets a wide variety of genes involved in diverse functions including cell cycle, growth arrest, DNA repair, metabolism, and apoptosis (Lee et al., Biotechnol Bioeng 50:273-279, 1995; de Belle et al., Oncogene 18(24):3633-3642, 1999; Virolle et al., J Biol Chem 278(14):11802-11810, 2003). Especially relevant to the current study is the finding that egr1 is induced in response to serum stimulation by quiescent or growing cells (Adamson et al., 2003). Thus the selection of cells with endogenous upregulation following serum withdrawal may help to explain the improved adaptation capacity of these particular cells to survive under reduced serum conditions. The upregulation of this gene in the adapted cell lines allows the cells to activate the genes needed for growth even in the absence of stimulation by serum factors. EGR1 expression is also associated with induced stresses such as hypoxia, emphysema, and vascular injury. The progressive reduction of serum also represents a stressful stimulus to the cells.

### Flow Cytometry and ELISA

The results presented herein indicate that cells transfected with either egr1 or gas6 appeared to adapt to serum free media more quickly than other cell lines. The effect of this gene expression on cell cycle progression, flow cytometric analysis of cells during exponential growth, propagated with 2% serum is shown in FIG. 18. At high serum levels, little difference was detected in the growth characteristics of cells expressing egr1 or gas6 relative to control cells. As the serum level was reduced from 10% to 2% for all cell lines, the fraction of cells present in the G0/G1 phases increased steadily from 52% to 78%. This result was consistent with reduced growth rates (FIG. 14) and longer times to confluency (FIG. 17). Expression of gas6 did not appear to alter the cell cycle progression of HEK-293 cells when compared with HEK-293 control cells even at low serum levels. At low serum levels expression of egr1 increased the fraction of HEK-293 cells in the G2/M phases by 8% as compared to the HEK-293 control cells. This increase in G2/M phase cells was consistent with improved growth rates and a reduced time to confluency for cells expressing egr1 relative to the control cells. To determine if the expression of either egr1 or gas6 affected heterologous protein production under serum-free conditions, an HEK-293 cell line expressing adipocyte complement-related protein of 30 kDa (acrp30) was assayed using ELISA. The resulting cell productivity values were essentially identical across the control cells and two transformed cell lines (Table 9, below).

TABLE 9
Results from ELISA for three different cell lines secreting acrp30.

| | | | Average specific |
|---|---|---|---|
| | | | protein production |
| Cell line | Viable cell density (10⁶ cells/mL) | Total protein production (ng) | $( \frac{ng \cdot mL}{cells} )$ |
| control with | 1.37 ± 0.03 | 1440 ± 170 | 1.05 × 10⁻³ |
| plasmid | | | |
| +gas6 | 1.29 ± 0.04 | 1370± 160 | 1.06 × 10⁻³ |
| +egr1 | 1.42 ± 0.04 | 1480 ± 220 | 1.04 × 10⁻³ |

Thus the inclusion of either egr1 or gas6 in these cells did not limit cell line productivity.

Results of the caspase activity did not indicate a difference in caspase 3 activity between cells expressing either egr1 or gas6 and control cells. However, it was observed that the longer cells were deprived of serum caspase 3 activity increased corresponding to an increase in apoptosis for some cells (data not shown). This supports earlier results indicating serum withdrawal reduces viability, as shown in FIGS. 15B and 16.

In the results presented herein, DNA microarrays were used to evaluate HEK-293 cells grown under varying serum levels in order to identify genes relevant to their adaptation to serum-free media (SFM). Clustering methods including self-organizing maps and principle component analysis were employed to group genes based on expression patterns. Only genes with expression levels consistently increasing or decreasing as the serum levels decreased were explored further. A number of genes, listed in Table 8, were identified based on gene ontology with particular emphasis placed on isolating genes with some record of promoting survival and growth or regulating cell cycle progression (Xiang et al., Curr Opin Drug Discov Devel 6:384-395, 2003; Fussenegger and Bailey, Biotechnol Prog 14:807-833, 1998; Lee et al., Biotechnol Bioeng 50:273-279, 1995). Two genes; egr1 (early growth response 1) and gas6 (growth arrest-specific 6), both of which exhibited increasing expression levels as the serum level decreased were selected for further study. The proteins these two genes encode are thought to be involved in a number of functions including survival, regulating growth, and other cellular processes such as signaling (Virolle et al., J Biol Chem 278(14):11802-11810, 2003; de Belle et al., Oncogene 18(24):3633-3642, 1999; Shankar et al., J Neurosci 26(21):5638-5648, 2006).

Egr1 encodes a protein consisting of 543 amino acid residues that localizes in the nucleus and belongs to a family of zinc-finger proteins commonly expressed in a variety of tissue including epithelial cells and lymphocytes (Fahmy et al., Nat Med 9(8):1026-1032, 2003; Liu et al., J Biol Chem 274(7):4400-4411, 1999). The protein EGR1 is a transcriptional regulator targeting numerous genes some of which are necessary for mitogenesis and differentiation (Fahmy et al., Nat Med 9(8):1026-1032, 2003). For instance, EGR1 regulates both transforming growth factor beta-1 (TGFβ1) and fibronectin by directly binding to and stimulating relevant promoters (Virolle et al., J Biol Chem 278(14):11802-11810, 2003; de Belle et al., Oncogene 18(24):3633-3642, 1999). Most recently, EGR1 expression was linked to signaling and association with the mitogen activated protein kinase (MAPK) pathway (Revest et al., Nat Neurosci 8(5):664-672, 2005).

In terms of functionality, EGR1 is thought to regulate the G0/G1 switch and interact with various cell cycle related genes like p21, cyclin D2, and p19. These properties are indicative of EGR1's relevance to cell cycle regulation and cellular growth. In addition, EGR1 is believed to have anti-apoptotic properties by inhibiting Fas (tumor necrosis factor receptor superfamily) expression and counteracting p53-dependent apoptosis (Virolle et al., J Biol Chem 278(14):11802-11810, 2003; de Belle et al., Oncogene 18(24):3633-3642, 1999). Specifically, EGR1 inhibits CD95 expression leading to insensitivity to FasL, a ligand triggering apoptosis (Virolle et al., J Biol Chem 278(14):11802-11810, 2003). Furthermore, human fibrosarcoma cells expressing EGR1 upon exposure to UV-C irradiation exhibited little apoptosis (de Belle et al., Oncogene 18(24):3633-3642, 1999). In fact, these cells were found to have elevated focal adhesion kinase (FAK) activity and reduced caspase activity; findings that are consistent with cell survival and reduced apoptosis.

The early growth response gene product (EGR1) localizes in the nucleus and belongs to a family of zinc-finger proteins involved in transcriptional regulation (Fahmy et al., Nat Med 9(8):1026-1032, 2003;Liu et al., J Biol Chem 274(7):4400-4411, 1999). EGR1 itself targets a wide variety of genes involved in diverse functions including cell cycle, growth arrest, DNA repair, metabolism, and apoptosis (Lee et al., Biotechnol Bioeng 50:273-279, 1995; de Belle et al., Oncogene 18(24):3633-3642, 1999; Virolle et al., J Biol Chem 278(14):11802-11810, 2003). Especially relevant to the current study is the finding that egr1 is induced in response to serum stimulation by quiescent or growing cells (Adamson et al., 2003). Thus the selection of cells with endogenous upregulation following serum withdrawal may help to explain the improved adaptation capacity of these particular cells to survive under reduced serum conditions. The upregulation of this gene in the adapted cell lines allows the cells to activate the genes needed for growth even in the absence of stimulation by serum factors. EGR1 expression is also associated with induced stresses such as hypoxia, emphysema, and vascular injury. The progressive reduction of serum also represents a stressful stimulus to the cells.

Interestingly, EGR1 has been found to be upregulated in the majority of human prostate tumors while its level has been low or absent from normal prostate tissue (Virolle et al., J Biol Chem 278(14):11802-11810, 2003). Furthermore, the level of egr1 increases with the degree of malignancy leading to belief that egr1 activity is critical to the survival and proliferation of these cells (Virolle et al., J Biol Chem 278(14):11802-11810, 2003). In support of the critical role egr1 plays in prostate tumors, studies in mice deficient of EGR1 experienced delayed prostate tumor progression and suppression of EGR1 also led to inhibition of angiogenesis and tumor growth (Virolle et al., J Biol Chem 278(14):11802-11810, 2003; Das et al., J Biol Chem 276(5):3279-3286, 2001; Fahmy et al., Nat Med 9(8):1026-1032, 2003).

As a transcriptional factor EGR1 regulates a multitude of targets including transforming growth factor beta-1 (TGFβ1), fibronectin, insulin-related growth factor II (IGF-II), platelet-derived growth factors A and B, epidermal growth factor (EGF) family members, and fibroblast growth factors (Fahmy et al., Nat Med 9(8):1026-1032, 2003; Virolle et al., J Biol Chem 278(14):11802-11810, 2003; de Belle et al., Oncogene 18(24):3633-3642, 1999; Adamson et al., 2003). EGR1 is also known to regulate various cell cycle related genes including p19, cyclin D2, p21, and MAD. EGR1 downregulates cell cycle inhibitors like p19, p21, and MAD allowing cells to progress through the cell cycle while also upregulateing cyclin D2 which controls the G1-S phase transition (Fahmy et al., Nat Med 9(8):1026-1032, 2003). Findings of the current study are consistent with a change in the cell cycle as HEK-293 cells expressing egr1 reached confluency more rapidly than control cells particularly at low serum levels and exhibited a higher fraction of cells in the G2/M phases.

EGR1 has also been linked to survivability and resisting apoptosis including downregulating caspase 7 (a member of the BH3 family), p53, and CD95 which binds the pro-apoptotic ligand FasL (Virolle et al., J Biol Chem 278(14):11802-11810, 2003; Das et al., Biol Chem 276(5):3279-3286, 2001; de Belle et al., Oncogene 18(24):3633-3642, 1999; Risbud et al., 2005). Thus, EGR1 has been proposed to desensitize cell death response in cells expressing it. Indeed, human fibrosarcoma cells expressing EGR1 exhibited little apoptosis upon exposure to UV-C irradiation (de Belle et al., Oncogene 18(24):3633-3642, 1999). In the present study, cells expressing the gene maintained higher viabilities under varying conditions (i.e. serum withdrawal and the adaptation process) and grew more rapidly at low serum levels than control cells.

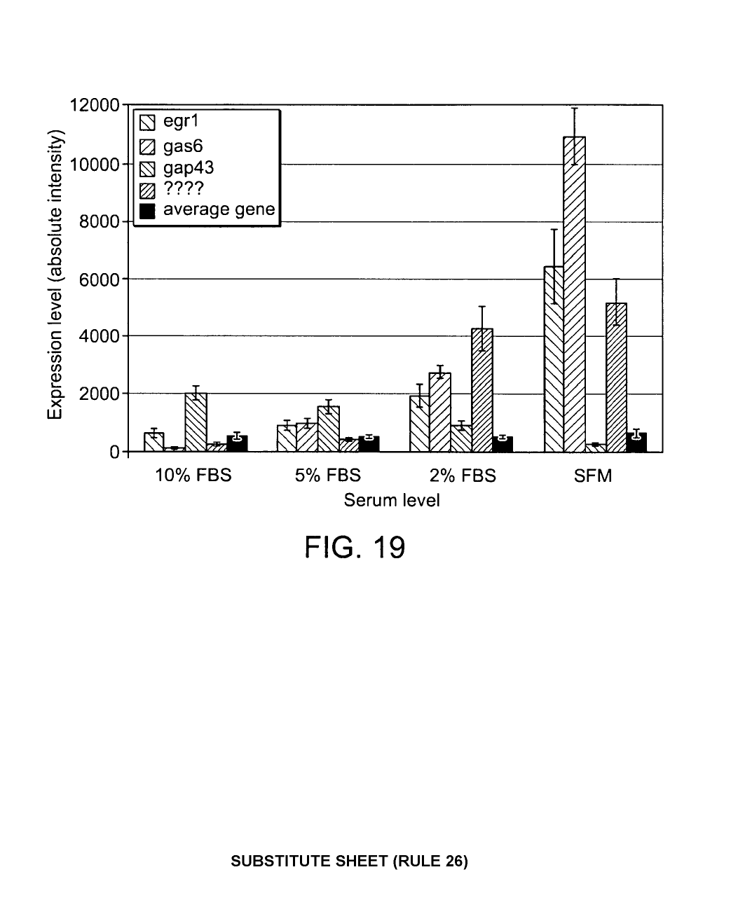

The expression of growth arrest specific 6 (gas6) was also increased as serum levels decreased in cells able to adapt to low serum levels (i.e. cells able to remain viable and progress through the cell cycle). (FIG. 19) This gene encodes a secreted protein that resembles Protein S, a common serum glycoprotein involved in the recognition and removal of apoptotic cells. GAS6, a vitamin-K dependent protein includes a number of N-terminal g-carboxyglutamic acid residues, four epidermal growth factor (EGF)-like domains, and two laminin-like domains (Munoz et al., Hum Mutat 23(5):506-512, 2004; Sasaki et al., EMBO J. 25(1):80-872006; Manfioletti et al., Mol Cell Biol 13(8):4976-4985, 1993).

Consistent with the findings in the current study, GAS6 was first identified in NIH 3T3 fibroblasts under serum starvation-induced growth arrest (Manfioletti et al., Mol Cell Biol 13(8):4976-4985, 1993). Recent studies have begun to elucidate the functional role of GAS6 as a ligand for the TAM family of tyrosine protein kinase receptors including ax1, sky, and mer (Stitt et al., Cell 80:661-670, 1995). The binding of GAS6 stimulates the tyrosine kinase activity of these receptors to trigger a down stream signal transduction cascade, often called the Gas6-Ax1 system (Stitt et al., Cell 80:661-670, 1995; Sasaki et al., EMBO J. 25(1):80-87, 2006). This ligand-receptor combination serves to regulate a range of functions including cell survival, growth, adhesion, phagocytosis, and migration (Shankar et al., J Neurosci 26(21):5638-5648, 2006; Stitt et al., Cell 80:661-670, 1995; Varnum et al., 1995).

GAS6 has been known to have an anti-apoptotic effect in a number of cell lines subjected to various stresses such as growth factor withdrawal or tumor necrosis factor-alpha toxicity (Shankar et al., J Neurosci 26(21):5638-5648, 2006; Lafdil et al., 2006). The anti-apoptosis activity is due to Akt phosphorylation which is activated by the Gas6-Ax1 complex. The presence of GAS6 has been shown to lower caspase 3 activity and increase Bcl-2 protein levels, promoting survival (Hasanbasic et al., Am J Physiol Heart Circ Physiol 287:H1207-H1213, 2004). Most recently, it was suggested that GAS6 led to an increase in phosphorylation of Bcl-2, resulting in inhibition of Bcl-2-antagonist of cell death (BAD) and caspase 3 (Son et al., Eur J Pharmacol 556:1-8, 2007). In the present work, the expression of gas6 also increased the percentage of viable cells at lower serum levels. HEK-293 cells expressing gas6 also exhibited enhanced growth indicated by a reduction in the time required for confluency as compared with the control cells. Previous studies have shown the involvement of GAS6 in stimulating the growth of muscle cells and fibroblasts in serum-free media (Stenhoff et al., 2004; Sainaghi et al., 2005). Therefore, the potential of egr1 and gas6 as enablers of adaptation to serum-free media in HEK-293 cells, are in line with the combined anti-apoptotic and growth stimulation effects of these genes uncovered in previous studies. Enhanced expression of either gene in HEK-293 cells increased growth rates and cell viabilities leading to improved adaptation capabilities. Given the ubiquitous nature of these genes and their functions, it is possible their influence may be seen across other cell types. Interestingly, microarray analysis revealed gash expression increased nearly 100 fold and map3k9 increased 20 fold from 10% to 0% serum while egr1 expression increased only 10 fold over this same range. However, egr1 expression appeared to provide the most significant enhancement in both cell viability and growth of the three genes. These findings suggest differences in expression levels are not the sole factor in selecting genes for investigation in cell engineering applications. The fact that EGR1 is a transcriptional regulator may have allowed for a more expansive control over growth and apoptosis than the secreted protein GAS6. Surprisingly, when both genes were upregulated, additive effects were not observed suggesting possible overlapping features or co-stimulation.

### Methods of the Invention

The results reported herein were obtained using the following Materials and Methods.

### Cell Culture Growth & Sampling

The two cell lines, anchorage-dependent and anchorage-independent HeLa cells, were initiated from frozen vials obtained from ATCC. Both cell lines were grown in BioFlo 3000 perfusion bio-reactors (New Brunswick Scientific) with a working volume of 5 L. Runs were conducted for up to 220 hours after inoculation with constant sampling to characterize growth parameters (Bleckwenn Biotechnol Bioeng 2005, 90:663-674). At least two different runs were carried out for each cell line. The media used was DMEM (Biosource), supplemented with 10% FBS (Bio source). The anchorage-dependent HeLa cells were grown on Cytodex 3 (Pharmacia) microcarriers. Each bio-reactor was seeded with 5.0×10⁵ cells/mL.

Samples from the bio-reactors were taken at regular intervals to test media composition (i.e. pH, Glucose, Lactate), cell viability, and density. For subsequent microarray analysis, samples were taken just prior to 150 hours after inoculation. Approximately 40 mL were taken from each bio-reactor and assayed for cell density. Cells were aligned using confluency (>90%) and deprivation of serum for 24 hours (Simon et al., Genet Epidemiol 2002, 23:21-26). A number of 2 mL RNase/DNase free micro tubes (Marsh Biomedical Products) were prepared from these samples such that each tube contained 5.0×10⁶ total cells. These samples were combined with TRIzol reagent (Invitrogen) and stored at −80° C.

### RNA Isolation & Probe Generation

Total RNA was isolated from samples using an Invitrogen kit (Micro-to-Midi Total RNA Purification System). Purified RNA was quantified using a GeneQuant Pro. Samples with an A_{260/280} ratio of at least 1.8 were then stored at −80° C. (Butte, 2002).

Each sample, consisting of approximately 10 μg of total RNA, was reverse transcribed according to standard protocol. Briefly, the following was added to each sample: 2 μL of random primers (Invitrogen, San Diego, Calif.), 6 μL of a commercially available 5× reverse transcriptase buffer, First Strand Buffer (QIAGEN GmbH, Hilden, Germany), 30 μL, of 0.1M dithiothreitol (Invitrogen, San Diego, Calif.), 1.2 μL of 25× aminoallyl-dNTP mix (Ambion, Austin, Tex.), and 2 μL of a commercially available reverse transcriptase, SuperScript II RT (Invitrogen, San Diego, Calif.). Samples were incubated at 42° C. overnight and hydrolyzed using 10 μL of 1M NaOH and 10 μL of 0.5M EDTA. The sample was dried in a speed vacuum and re-suspended in 0.1M Na₂CO₃ buffer (pH 9.0). 4.5 μL of bright fluorescent dye, NHS-Cy3 was added to the mixture, followed by incubation for 1 hour in the dark, at room temperature. NHS-Cy3 was used to label the control samples (fluorescent λ=532 nm) and NHS-Cy5 was used to label the test samples (fluorescent λ=635 nm). Selectively binding membranes and wash buffers in a commercially available kit, Qiagen PCR Purification kit was used to remove uncoupled dye. A single control sample and a single test sample were then mixed together and allowed to dry to completion in a Speed Vac, which is a concentrator. The labeled probes were re-suspended in 24 μL of preheated 1× hybridization buffer. Additionally, 14, of competitor DNA, COT1-DNA, and 1 μL, of Poly(A)-DNA was added to the mixture. The probe mixture was then heated at 95° C. for 3 minutes before being snap cooled on ice for 30 seconds (Quackenbush, supra).

cDNA Microarray Preparation, Hybridization, Analysis, And Subsequent Verification

High quality microscope slides (Corning) were printed with a set of 32,448 ESTs by The Institute for Genomic Research (TIGR) using an array fabricator (Intelligent Automation). Prior to hybridization, the slides were washed in a buffer comprising sodium chloride and sodium citrate, 5×SSC (Invitrogen, San Diego, Calif.), 0.1% SDS (Invitrogen), and 1% bovine serum albumin (Sigma) for 45 minutes at 42° C. Next, the slides were washed in de-ionized water at room temperature, followed by a wash in 100% isopropanol at room temperature.

Each prepared sample was approximately 26 μL in volume and was pipetted at one end of the slide. The hybridization chamber was then wrapped in foil and incubated overnight in a water bath at 42° C. After washing and drying, each slide was ready for scanning and image analysis using a microarray imager, the GenePix 4000B imager (Molecular Devices, Sunnyvale, Calif.). To ensure proper labeling efficiency dye-swapping experiments were performed with several arrays. Image analysis software, Acuity software (Molecular Devices, Downingtown, Pa.) was used to normalize and mine the data. Normalization was accomplished by applying a series of criteria including circularity, flags, and signal intensity relative to local background (Xiang et al., Curr Opin Drug Discov Devel 6:384-395, 2003). Total intensity and regression models were applied to normalize microarray data (Burke, Mol Diagn 2000, 5:349-357; Conway and Schoolnik, Mol Microbiol 47:879-8892003).

Once normalization was complete, the data was filtered in order to identify reproducible (n≧3) genes with differential expression (i.e. expression ratios 1). In all, 4 cDNA microarrays fabricated by TIGR and 2 pairs of oligonucleotide arrays from Affymetrix were hybridized with our RNA samples. A protocol for preparation and hybridization of Affymetrix arrays is commercially available.

To verify the results of the cDNA microarray experiments, hybridization was performed with 2 pairs of oligonucleotide arrays (Affymetrix), followed by RT-PCR on the two genes selected. Standard protocols were used for both procedures and are readily available online from various manufactures (e.g. Affymetrix, Applied Biosystems, Ambion). Each gene was assayed four times through two separate RT-PCR experiments to establish greater statistical significance.

Over-Expression & siRNA Studies

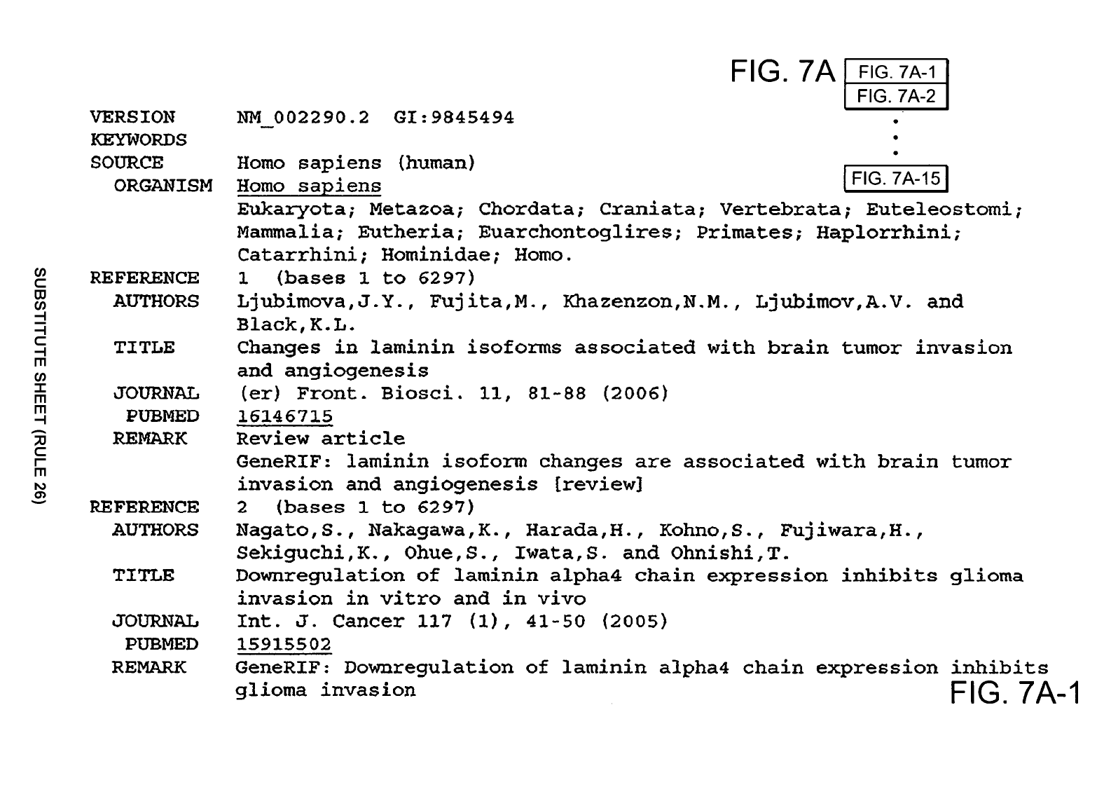

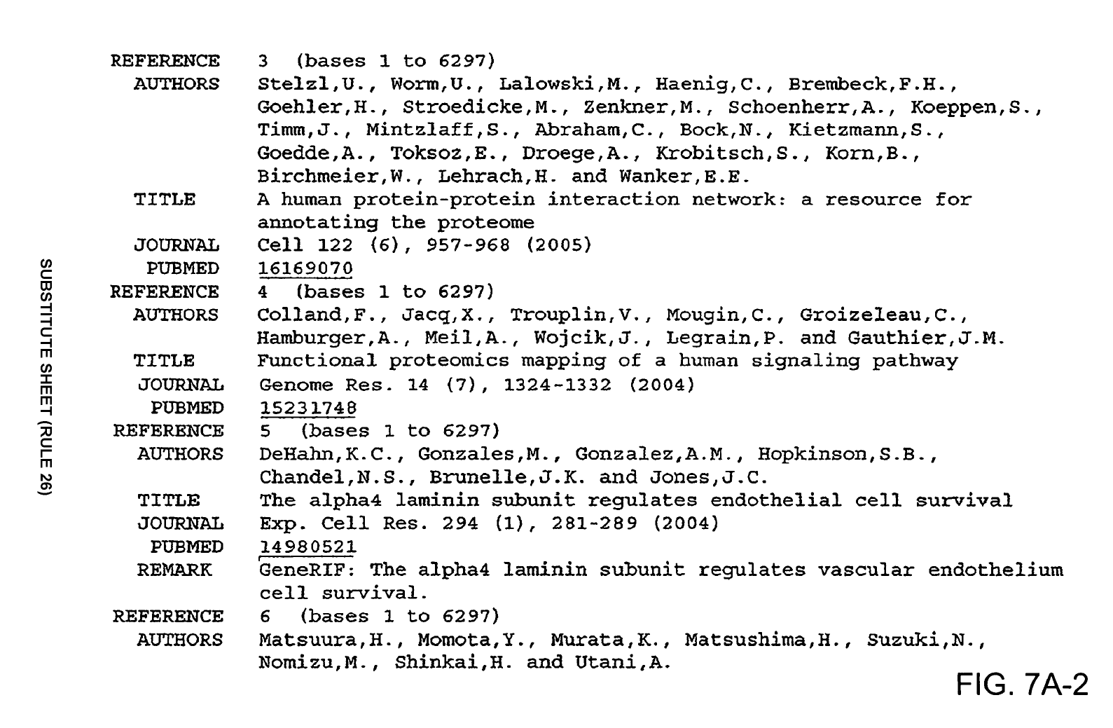

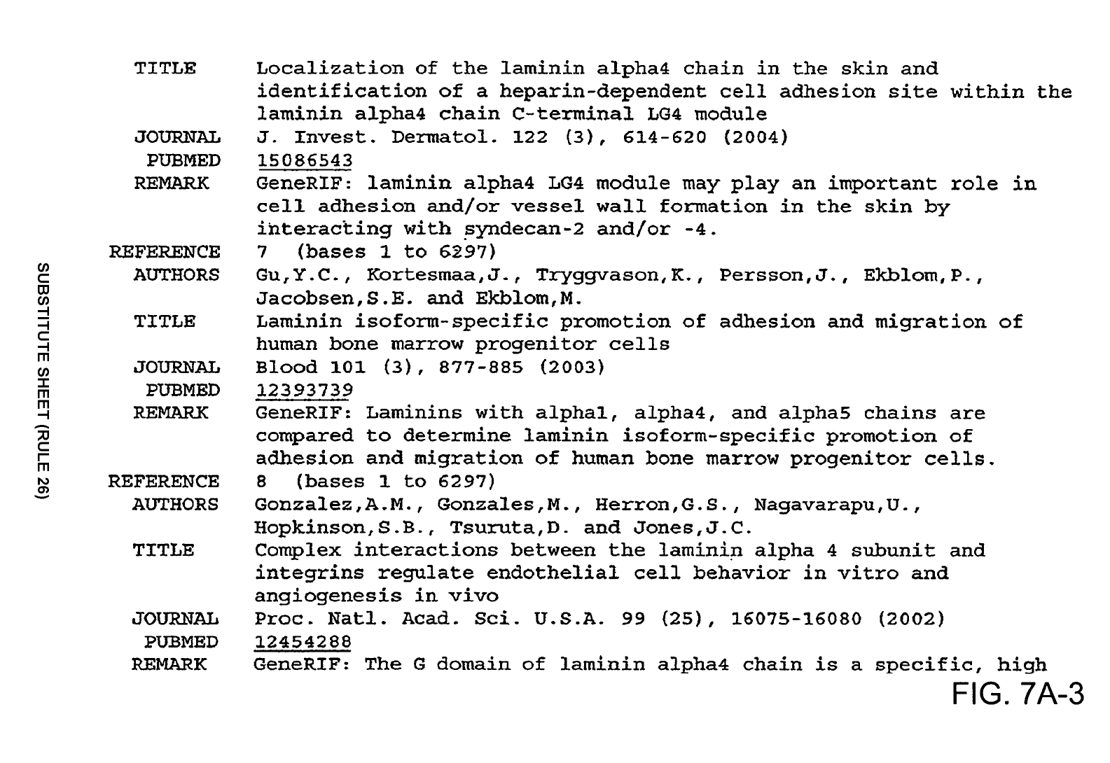

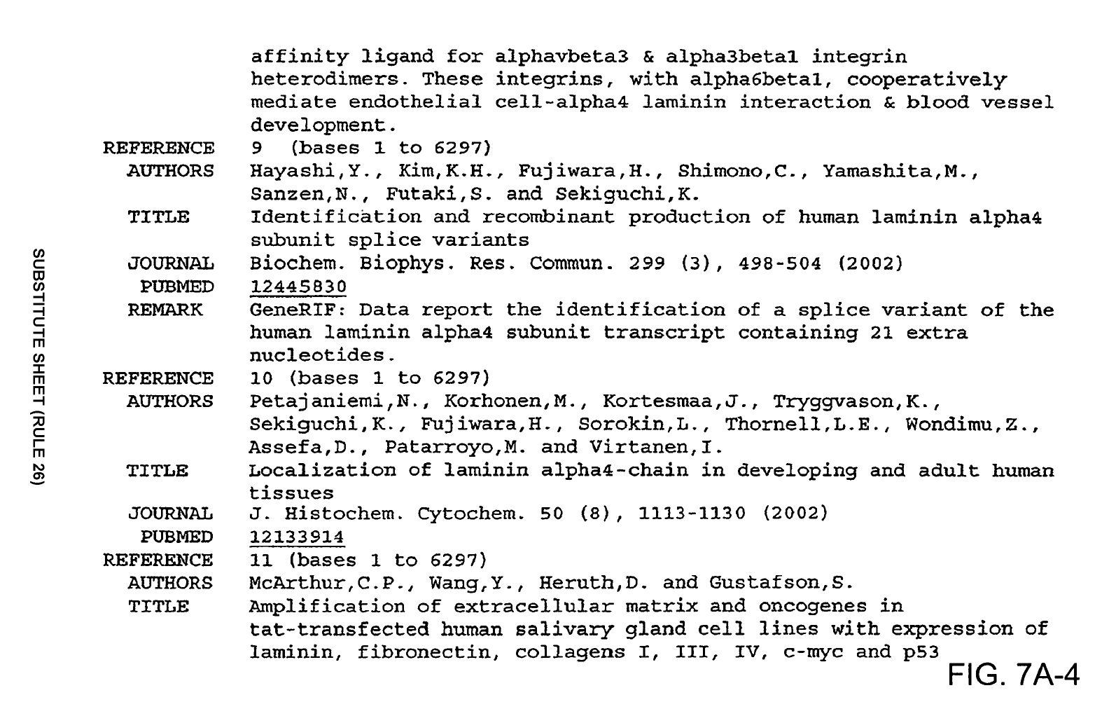

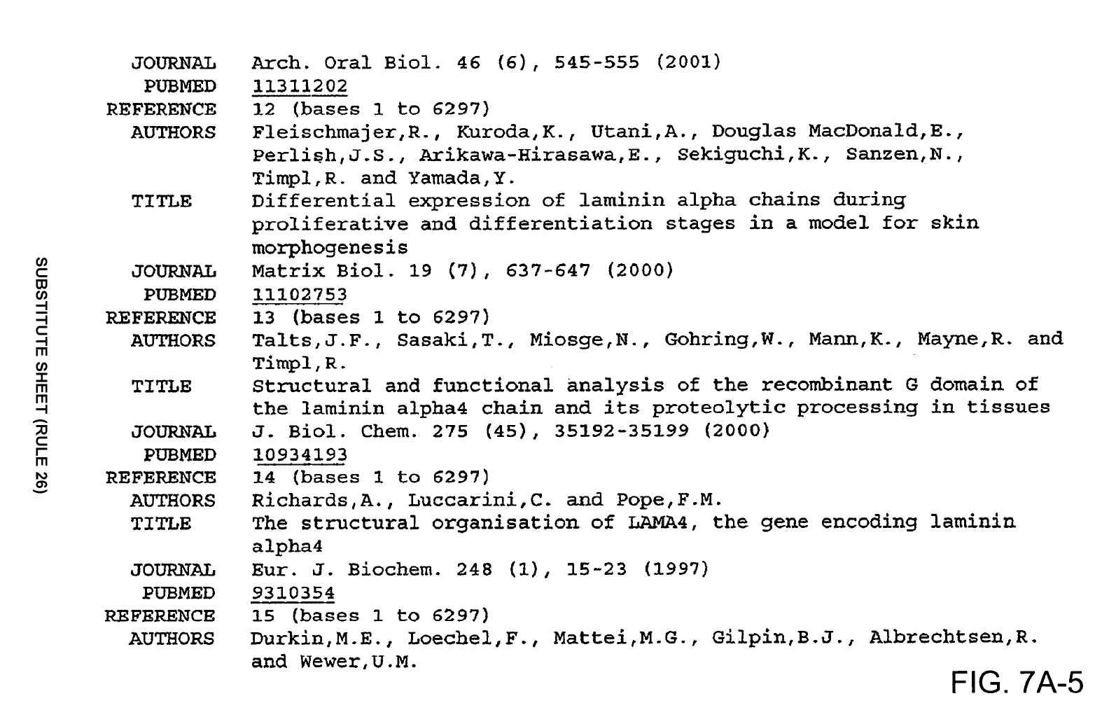

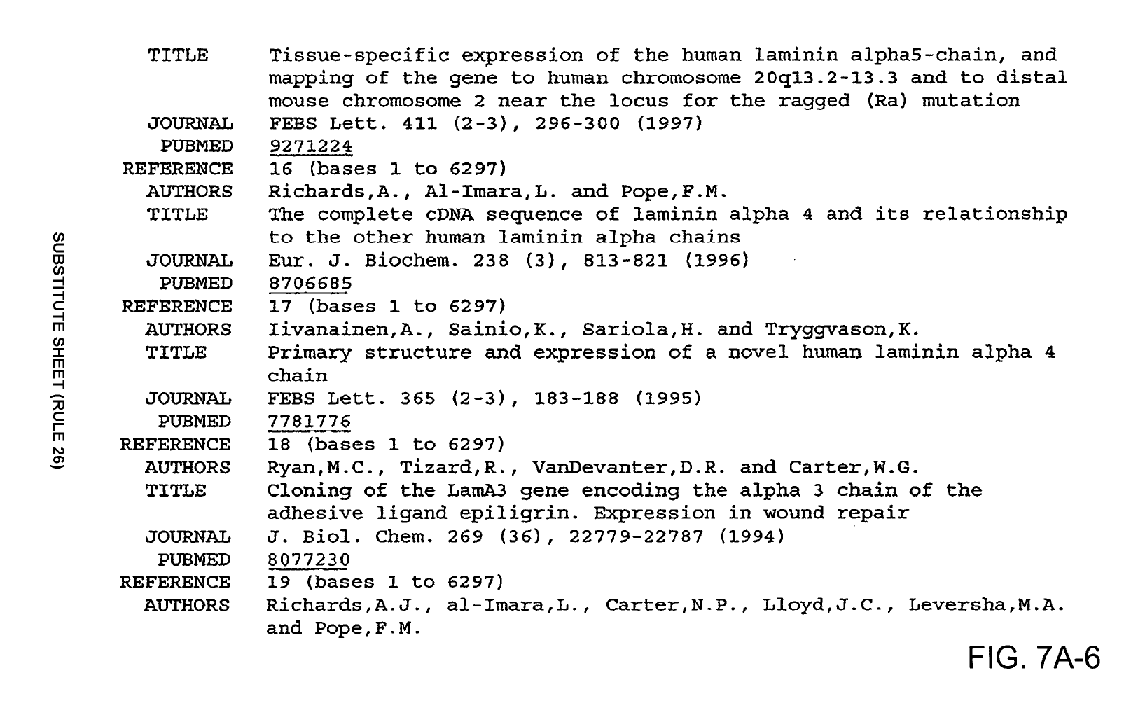

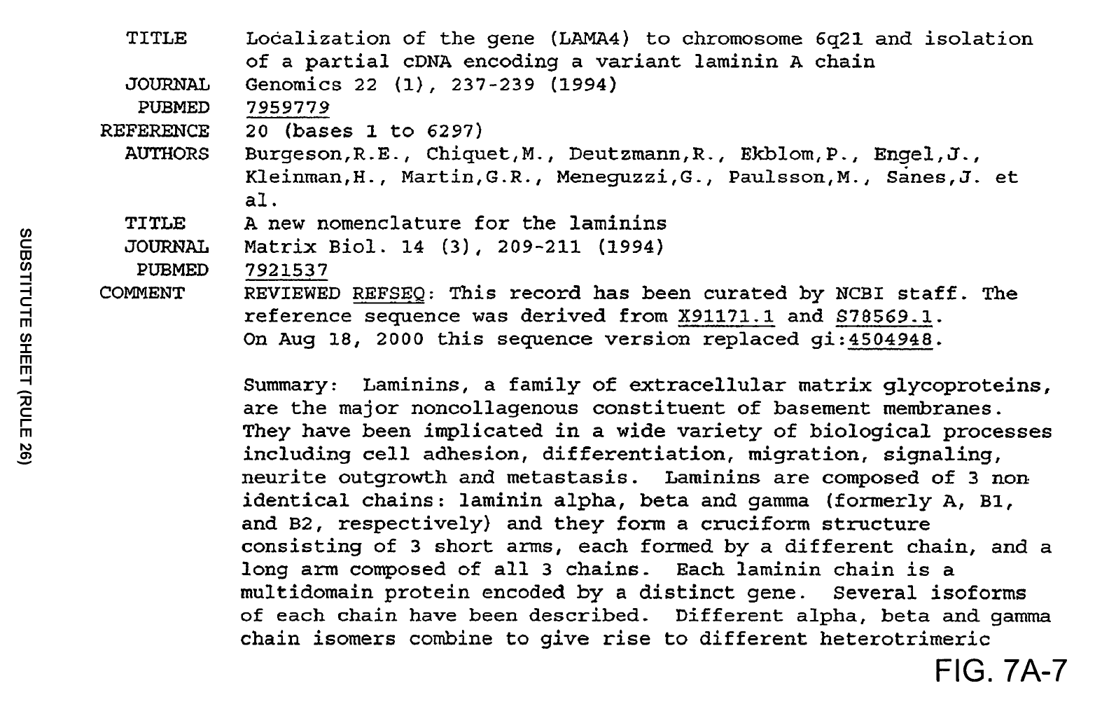

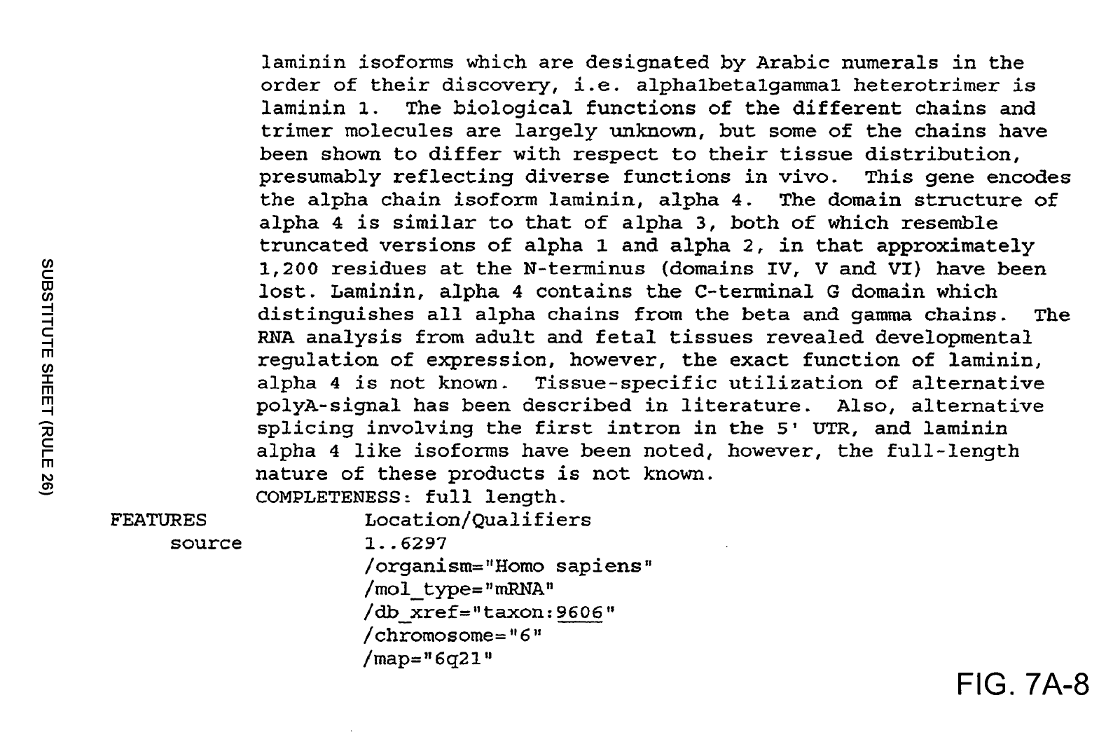

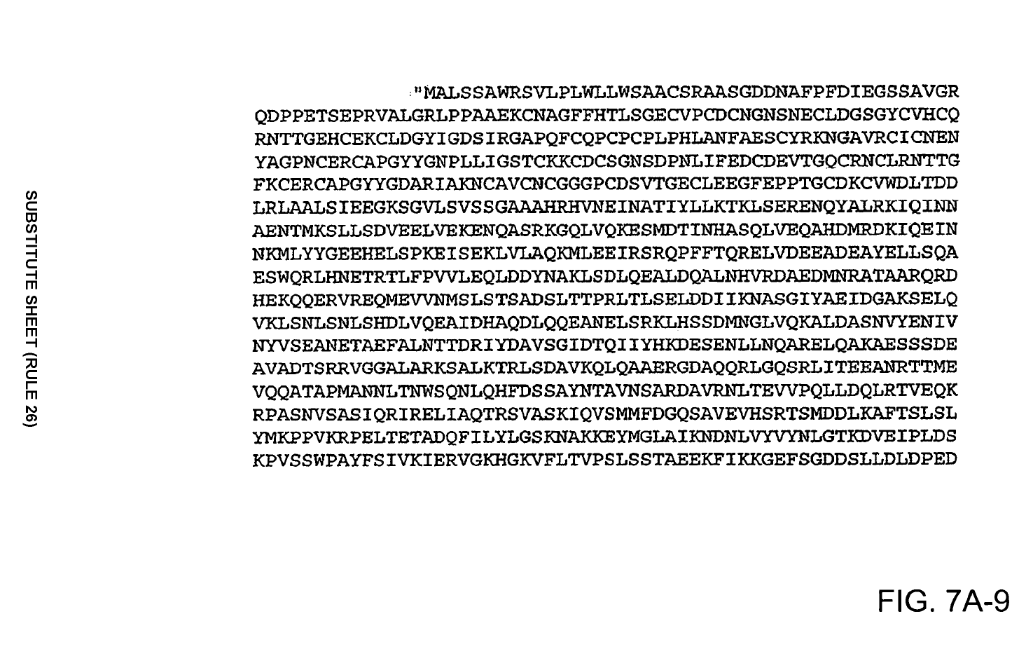

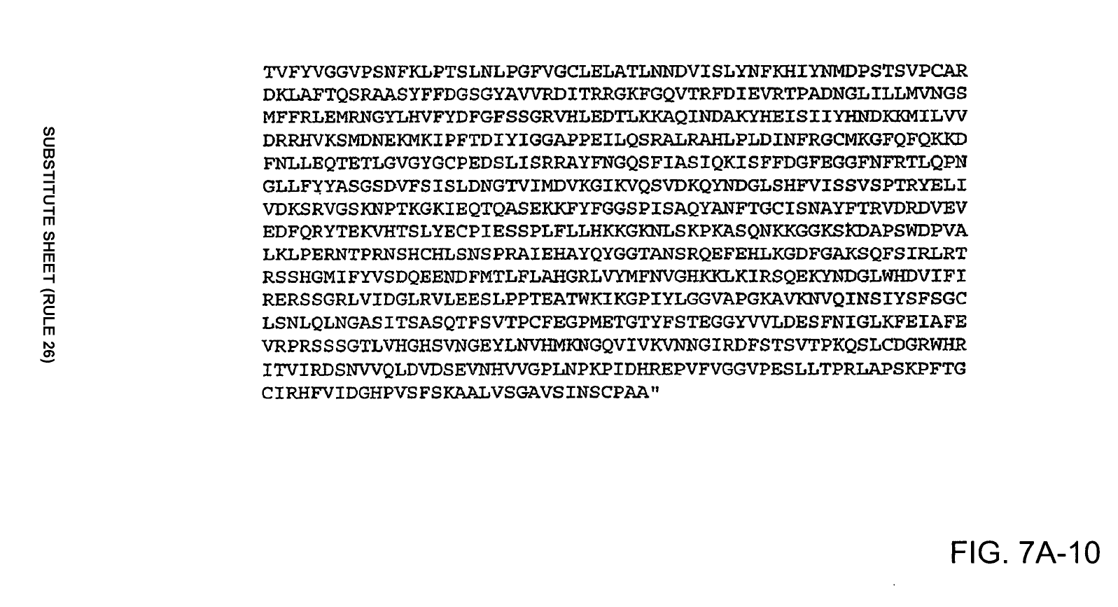

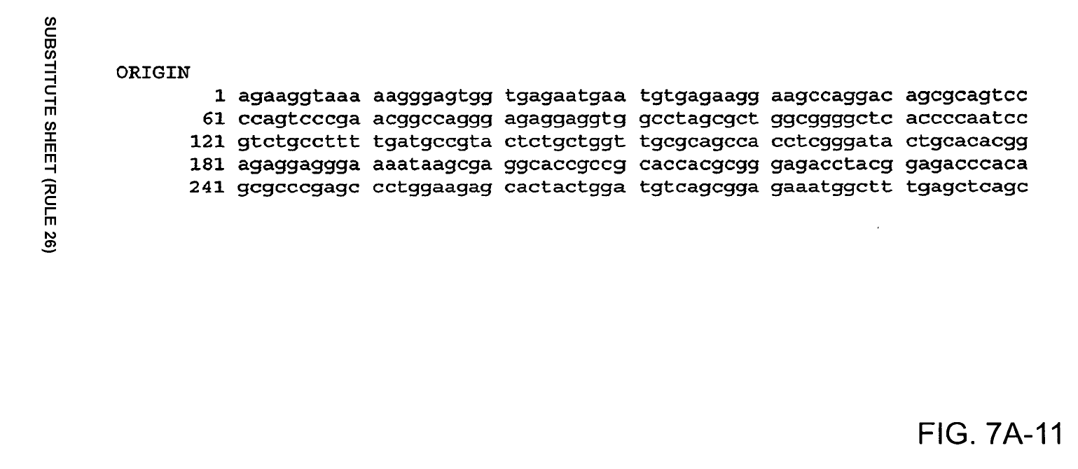

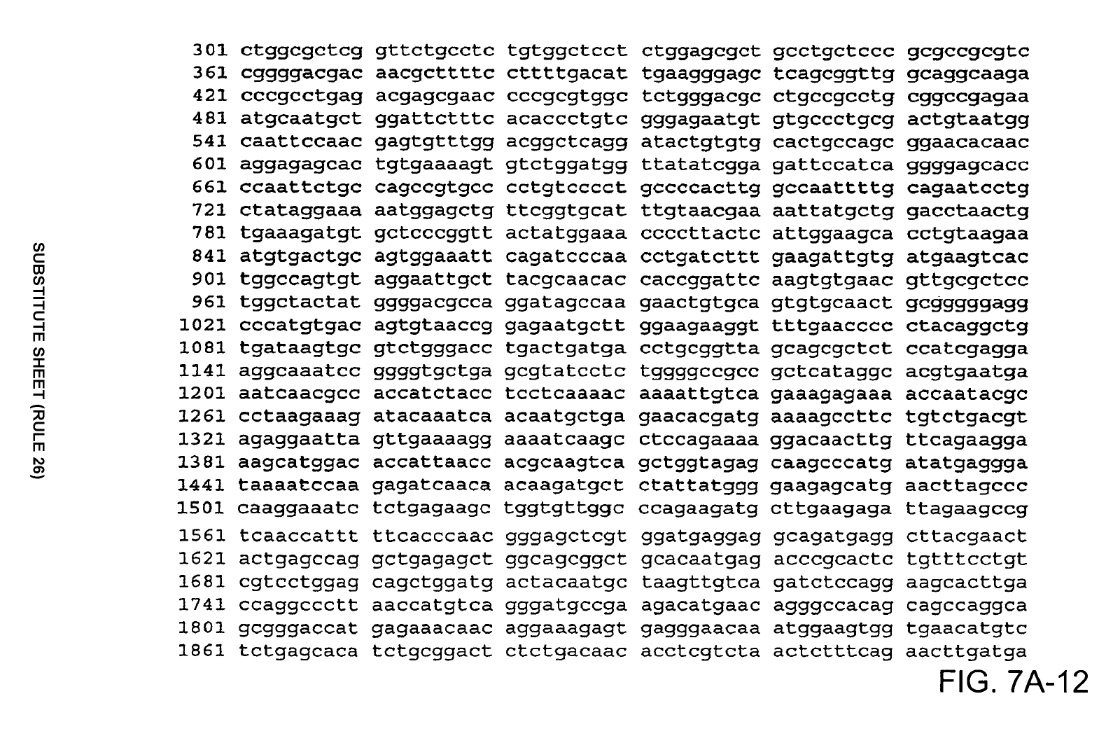

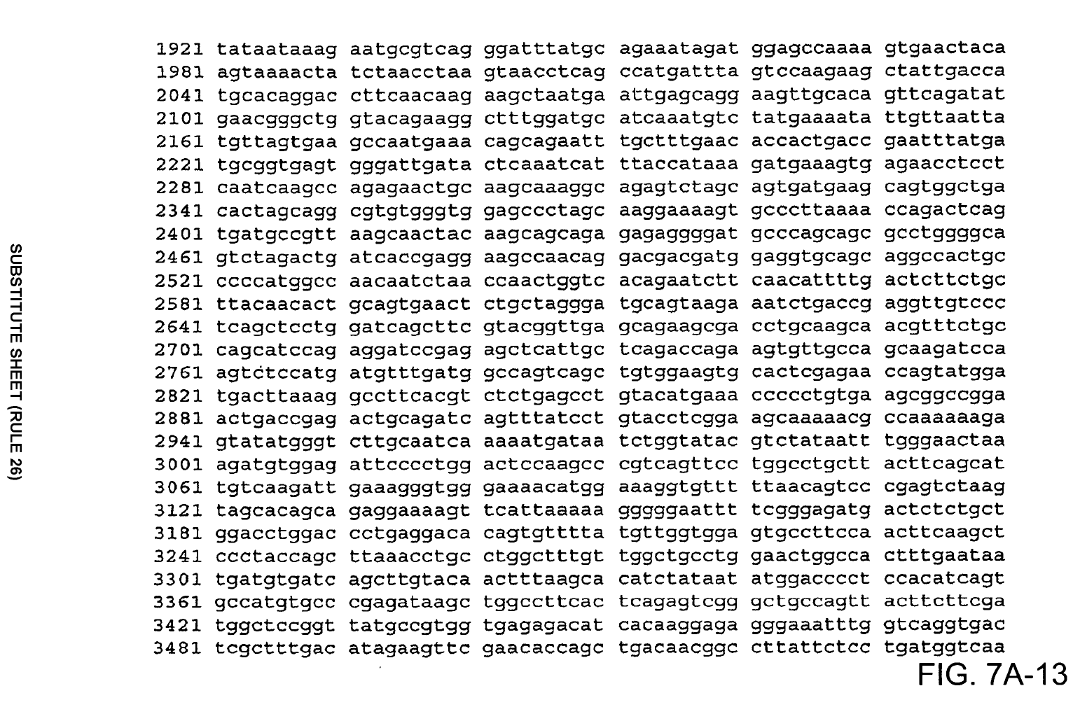

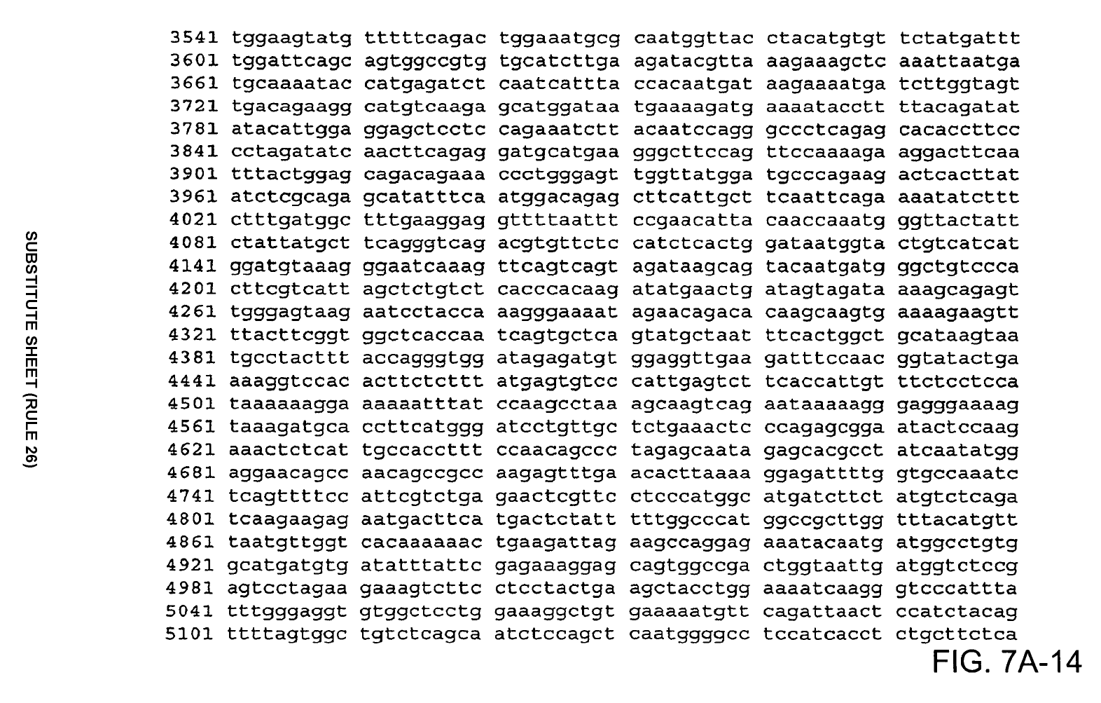

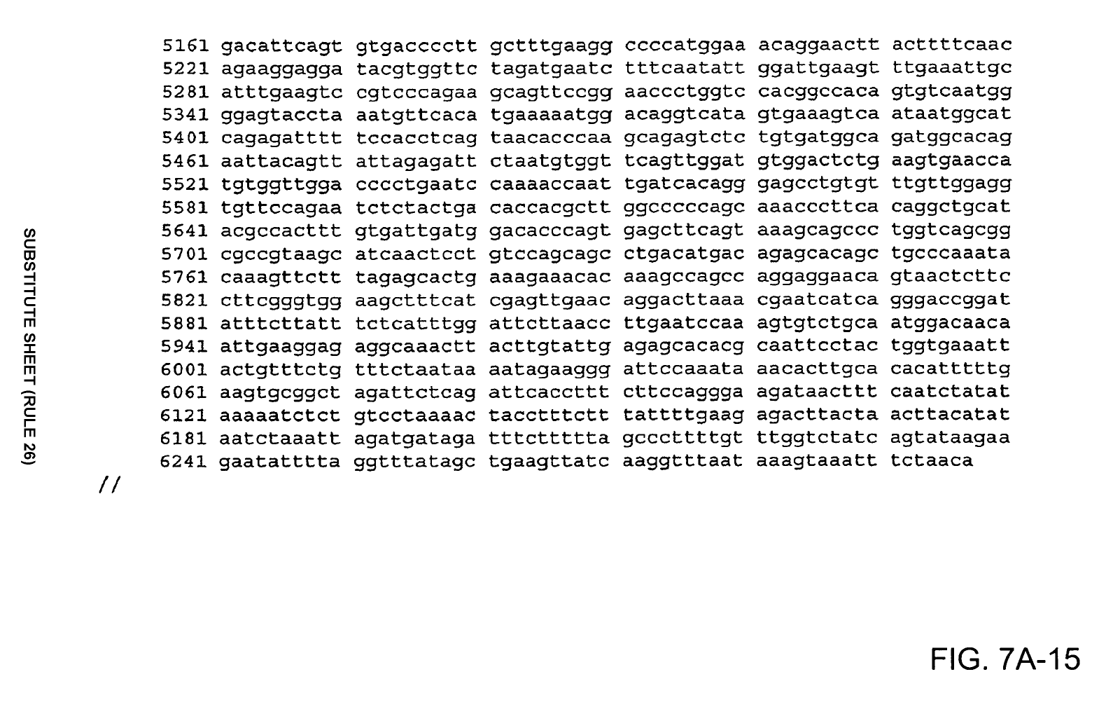

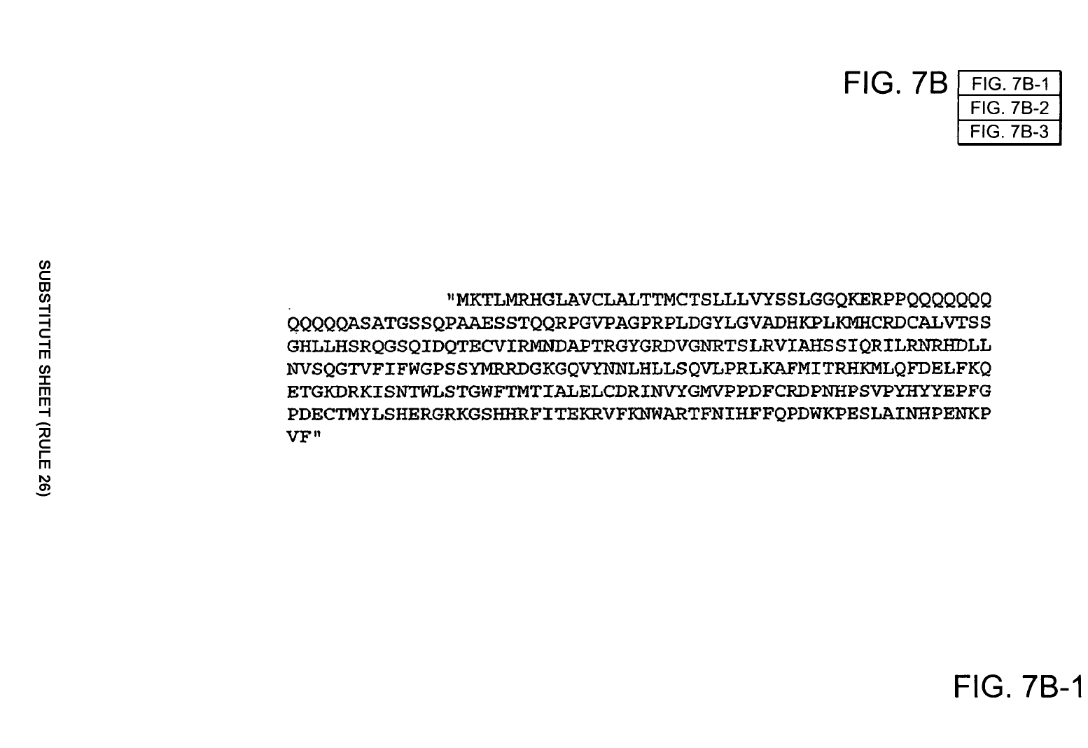

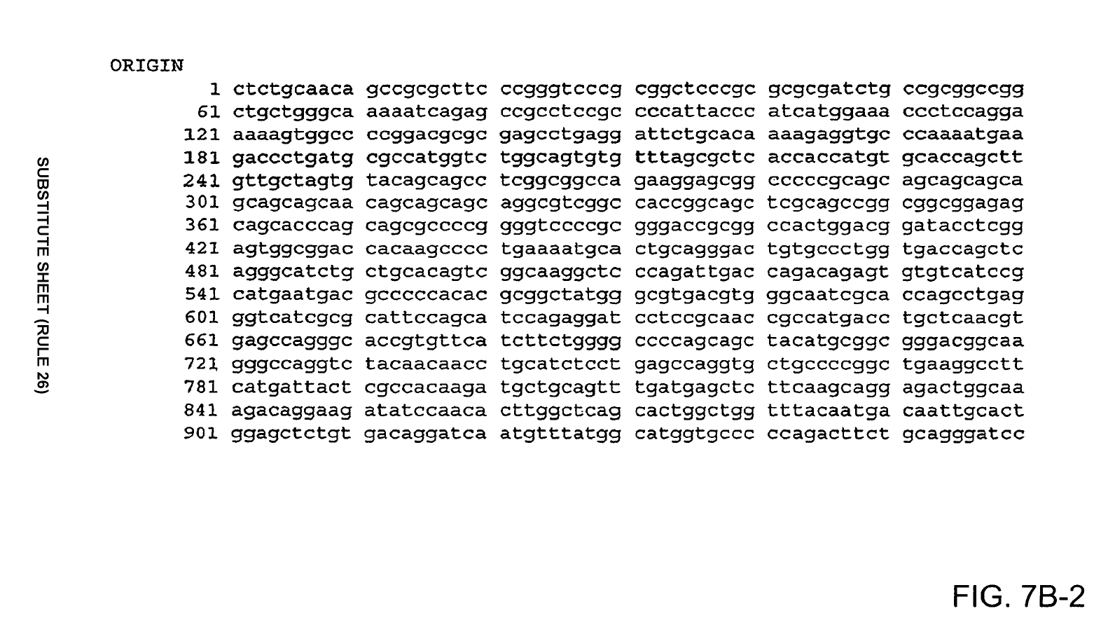

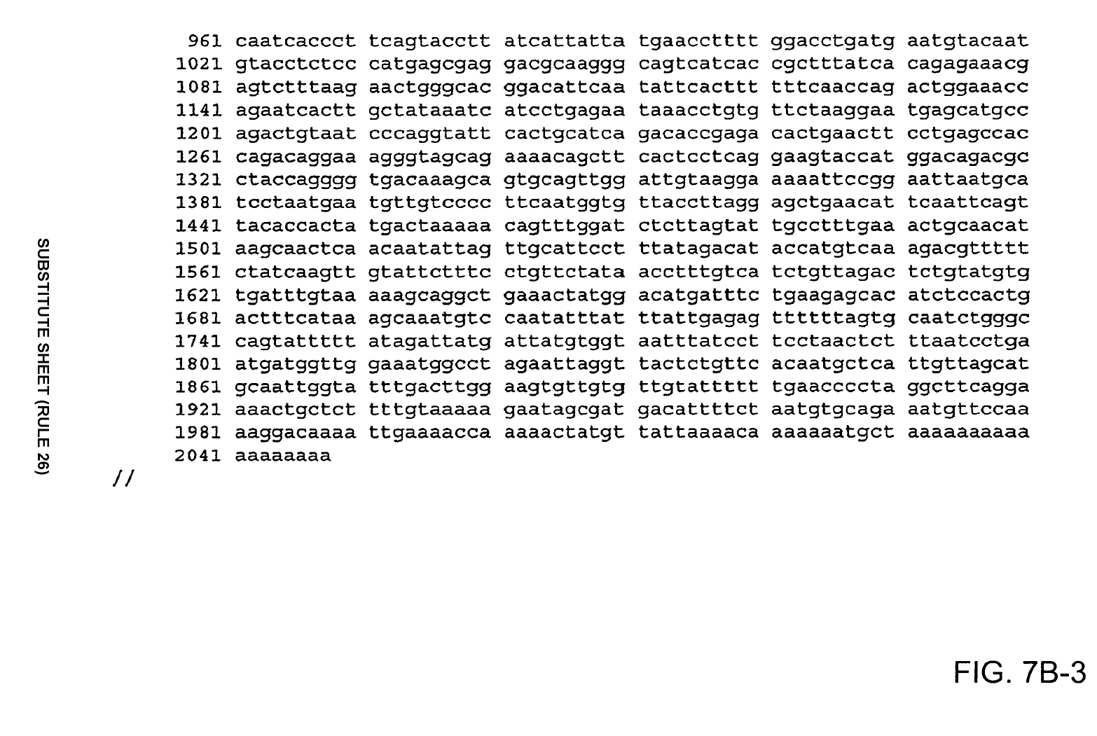

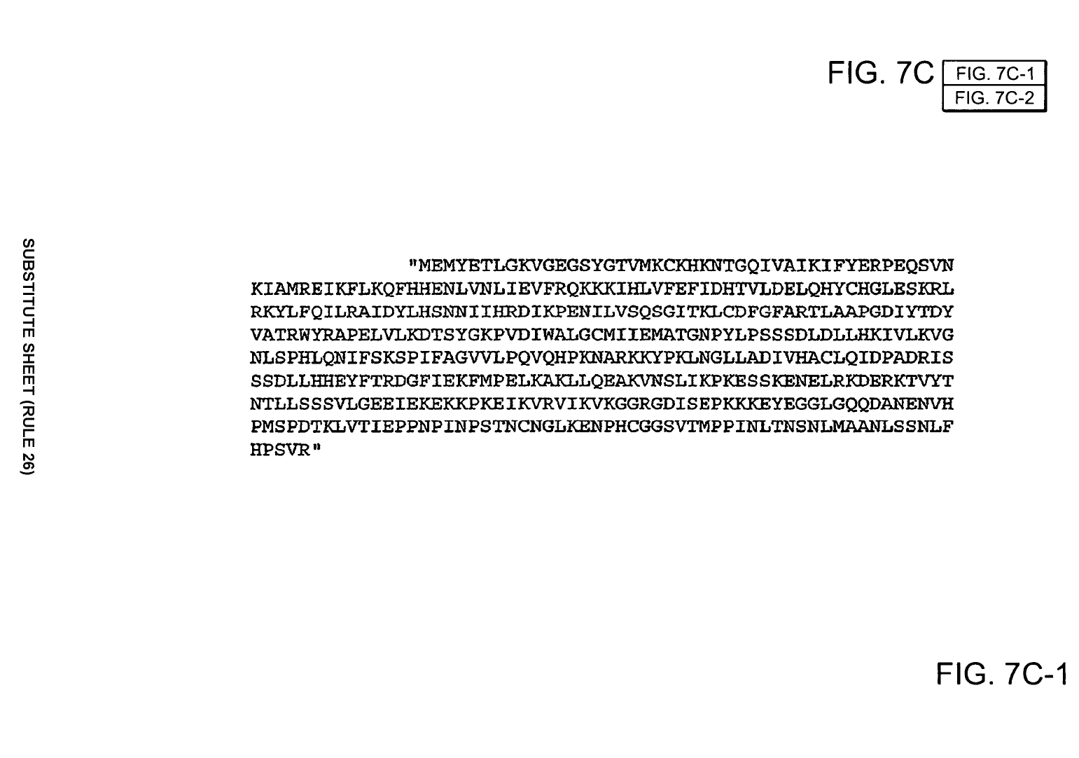

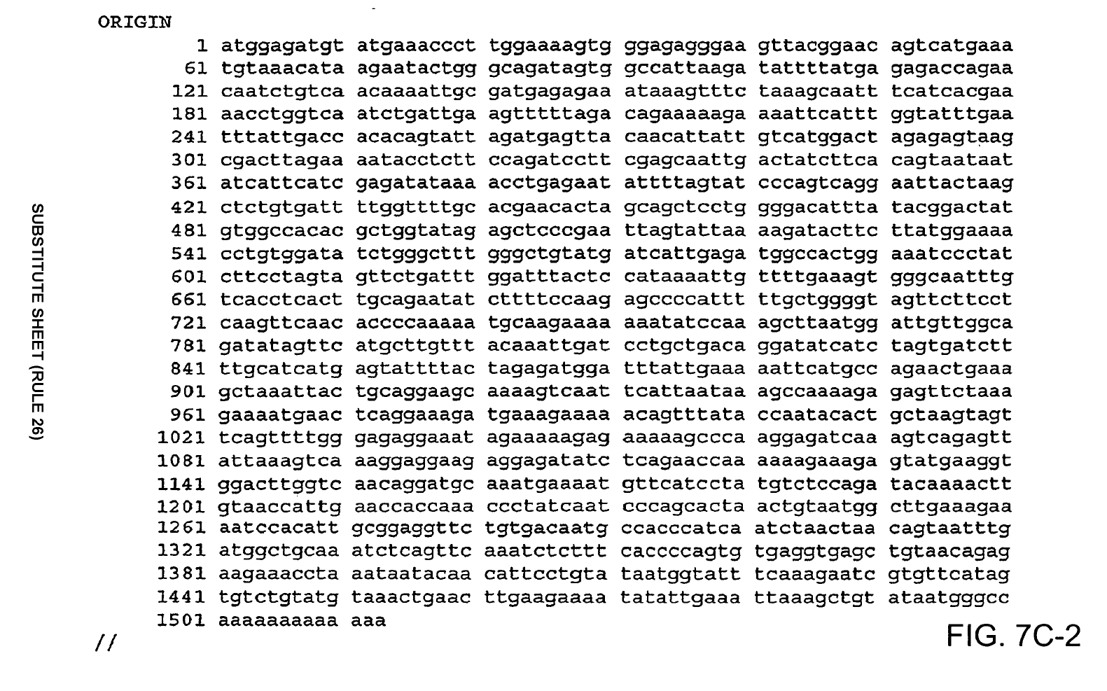

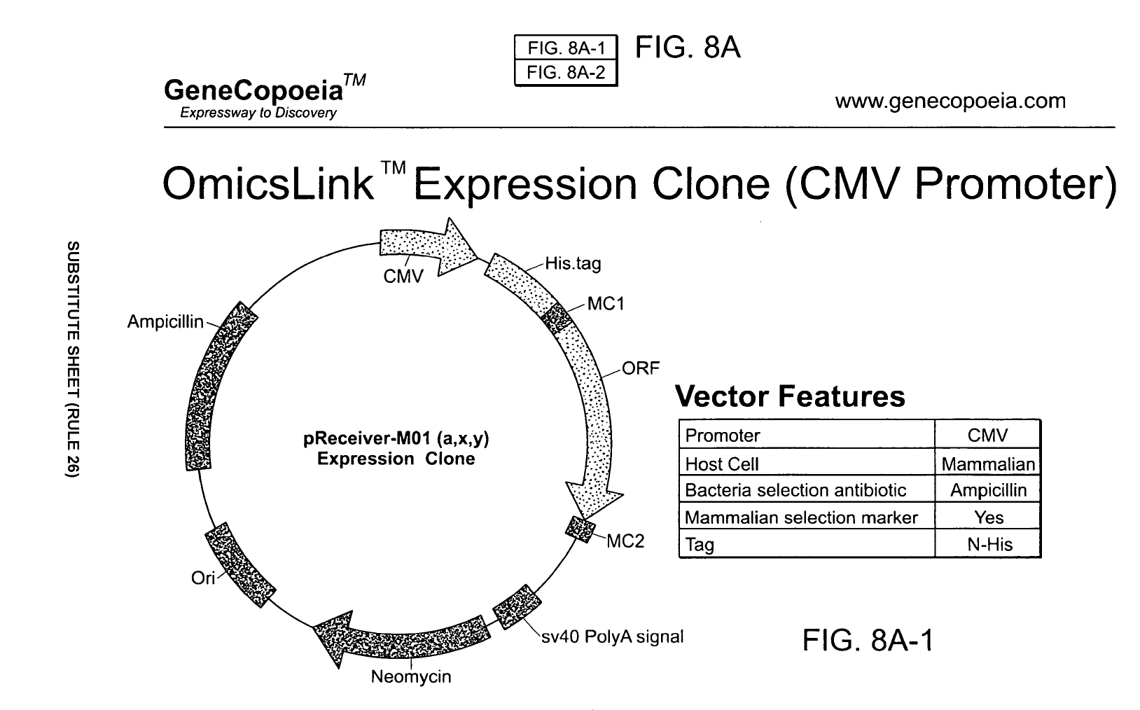

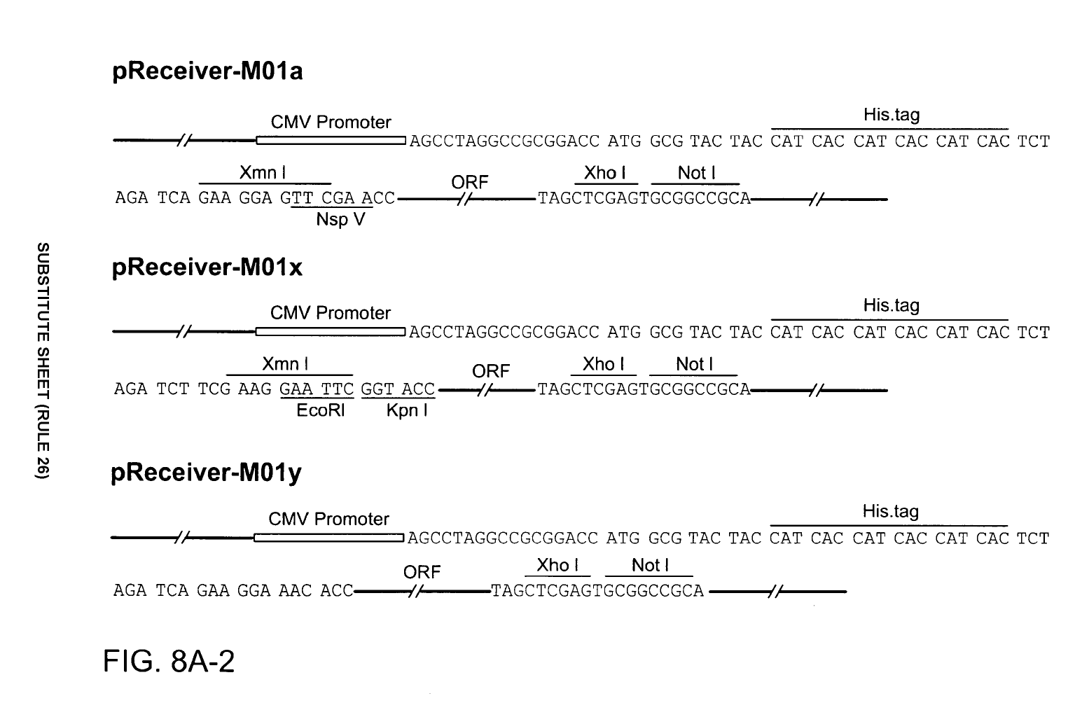

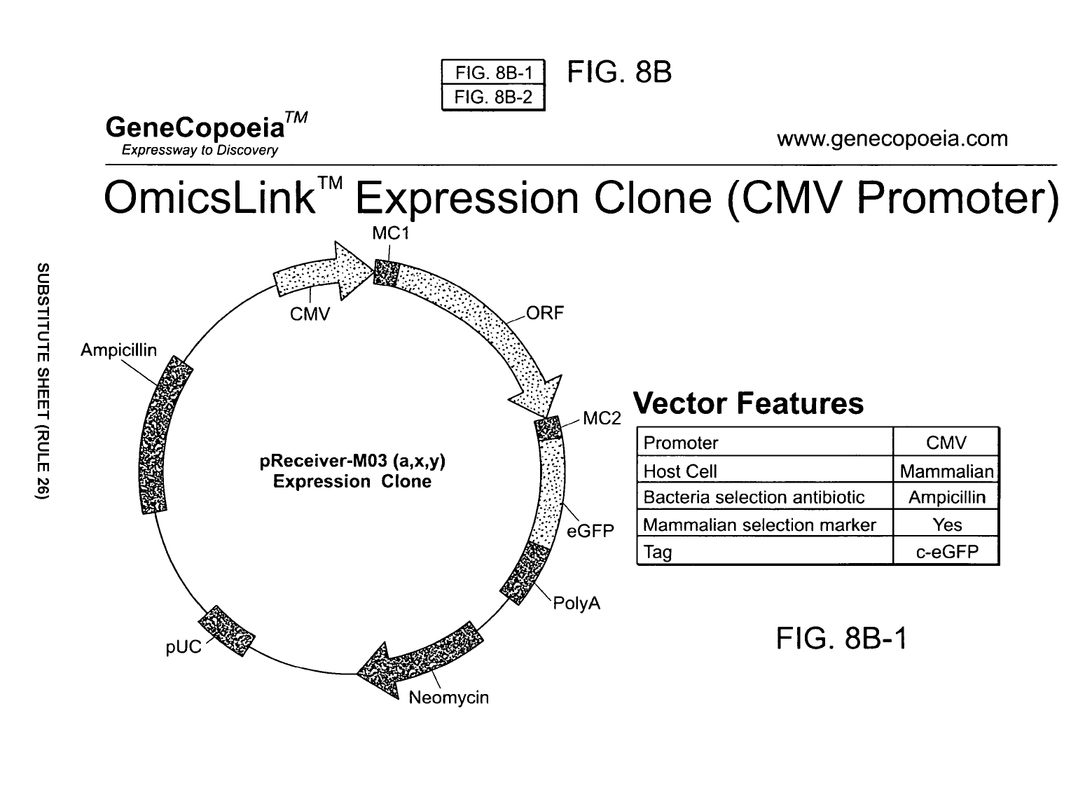

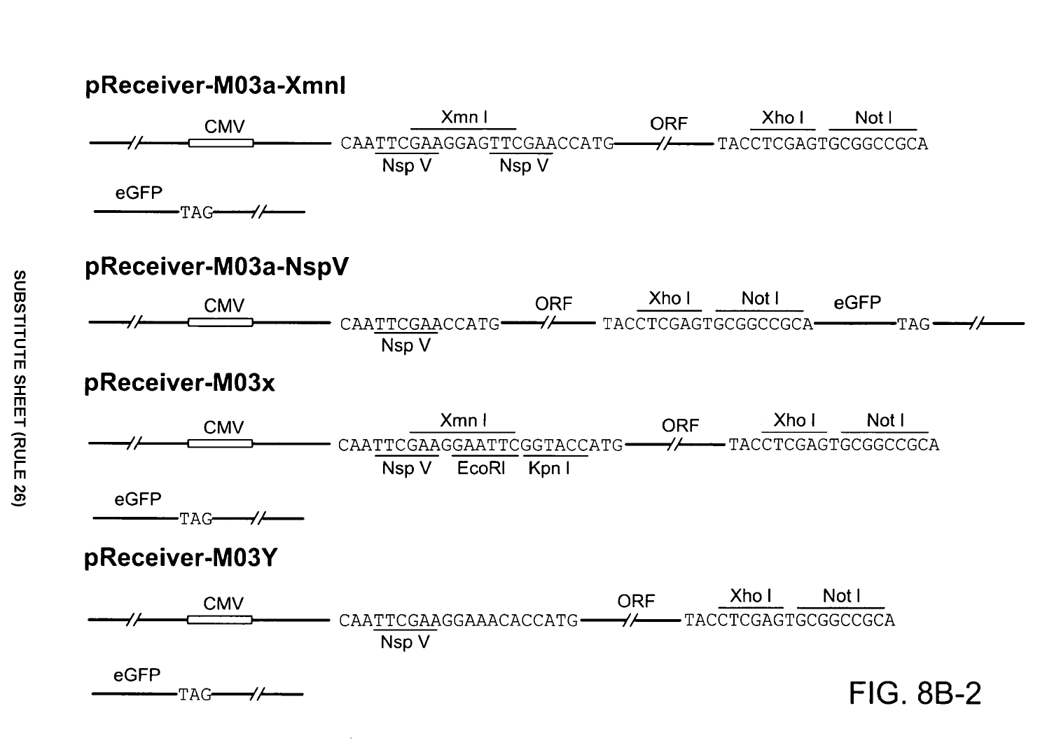

Using public online databases, the DNA and amino acid sequence for both siat7e and lama4 was obtained. For each gene, full-length DNA constructs (GeneCopoeia) and small interfering RNA (siRNA) (Invitrogen) were purchased. The full-length gene sequences (FIGS. 7A-7C) were contained in expression vectors (FIGS. 8A and 8B) that could be transfected into mammalian cells. Several siRNA sequences were constructed using a combination of online vendor programs and software packages. The sequences used were as follows:

Lama4:
5′-AUU GUA GUC AUC CAG CUG CUC CAG G-3′
Siat7e:
5′-GGA AGU UGC ACA GUU CAG AUA UGA A-3′

Manipulations were designed using transfection protocols available from Invitrogen. Starting with a frozen vial (or existing cell line) cells were expanded into two T-150 flasks. Once T-150 flasks were approximately 90% confluent, cells were moved into two 6-well plates (one 6-well plate per T-150 flask) with fresh media. After one day, when the cells were greater than 70% confluent, cells were transfected using Lipofectamine 2000 and gene-specific siRNA/RNAi (200 μmol of siRNA/RNAi and 5 μL of Lipofectamine 2000) in 2 mL of media. After 6 hours, the media was replaced with fresh media. Samples were drawn after 24-48 hours to verify successful transfection using RT-PCR.

Physiological changes, post-transfection, were observed and then recorded with a Leica DM IRB microscope and attached camera. Quantification of observable changes was performed using a commercially available cell counting, particle counting, and particle size analysis device, the Z Series COULTER COUNTER, Beckman Coulter (Fullerton, Calif.). In order to assay the level of adhesion for a specific manipulation, the formation and distribution of cellular aggregates was analyzed in both control (untreated) cells and treated cells. Samples were diluted in an electrolyte solution at a ratio of 1:2 and run through the Beckman Coulter counter for varying sizes. All controls yielded similar results (i.e. ≦4% variation between different control samples for each size range) and therefore were averaged together and labeled ‘control’. In the experiments, several different controls were employed. The first control consisted of normal cells supplied with complete media, however, unaltered in anyway. Another control consisted of cells incubated with only the transfecting agent Lipofectamine 2000 and complete media. Another control consisted of cells transfected with control siRNA (i.e. nonsense siRNA). A final control consisted of cells transfected with just the expression vector (no siRNA or gene insert). Samples were diluted in an electrolyte solution at a ratio of 1:2 and run through the Beckman Coulter counter for varying sizes. All the controls yielded similar results (i.e. 4% variation between different control samples for each size range), and therefore were averaged together and labeled ‘control’.

Because typical anchorage-independent HeLa cells range in size from 12-15 μm, the ranges chosen (in μm) can be thought of as follows: <5 (debris & cellular fragments-background), 5-15 (single cells), 16-30 (dividing cells & clusters of 2 cells), 31-60 (large clusters of 2 cells and clusters of 3-4 cells), 61-80 (clusters of 5-6 cells), 81-100 (clusters of 7-8 cells), and >100 (clusters of 8 or more cells) (Masters, 2002).

### Creation of Stable Cell Lines & Attachment Assay

Four stable cell lines were created, each constantly expressing a particular gene to a greater or lesser extent than its originating cell line. The names of these new cell lines are: anchorage-dependent [lama4−], anchorage-dependent [siat7e+], anchorage-independent [lama4+], and anchorage-independent [siat7e−]. The first two cell lines were prepared using anchorage-dependent HeLa cells. In the case of the anchorage-dependent [lama4−] cells, the expression of lama4 was blocked by inserting a vector (GeneScript) embedded with siRNA specific for lama4. Anchorage-dependent [siat7e+] cells were prepared by inserting an expression vector that contained both the full-length sequence of the gene siat7e and a CMV promoter (GeneCopoeia). Similarly, anchorage-independent [lama4+] cells from anchorage-independent HeLa cells by introducing an expression vector that contained the full-length lama4 gene. The anchorage-independent [siat7e−] cells were also prepared from anchorage-independent HeLa cells using a vector that contained siRNA specific for siat7e. The selection of stable transfectants relied upon additional components of the expression vectors used such as resistance genes or selection markers (Bohm et al., 2004; Dopazo et al., 2001; Datta and Datta, 2003).

To quantify adhesion properties in these modified cell lines, a shear flow chamber was used. The apparatus was placed onto the stage of an inverted microscope with a camera capable of generating real-time photos and video. By growing cells on a 35 mm×10 mm cell culture dish (Corning) the shear chamber was placed inside the culture dish. Thus, varying levels of shear stress were introduced using Hanks' based, enzyme-free cell dissociation buffer (Gibco), past the adherent cells on the culture dish. Preliminary experiments established the range of shear stresses applicable to the cell lines. Subsequent experiments quantified each cell line's adhesion characteristics based on the number of cells that would detach for a given period of time and a given shear stress. The number of cells detaching during the course of a single experiment was converted into percentages using the initial number of cells present in the viewable region. Shear stress values of 19.2, 24.0, and 28.8 dyn/cm² were used, each for one minute in succession for a single cell culture dish. At least three separate dishes were assayed for a given cell line to establish statistical parameters.

Anchorage-independent and anchorage-dependent HeLa cells, displaying markedly different growth characteristics, were analyzed using DNA microarrays. Two genes, cyclin-dependent kinase like 3 (cdkl3) and cytochrome c oxidase subunit (cox15), were up-regulated in the faster growing, anchorage-independent (suspension) HeLa cells relative to the slower growing, anchorage-dependent (attached) HeLa cells. Enhanced expression of either gene in the attached HeLa cells resulted in elevated cell proliferation, though insertion of cdkl3 had a greater impact than that of cox15. Moreover, flow cytometric analysis indicated that cells with an insert of cdkl3 were able to transition from the G0/G1 phases to the S phase faster than control cells. In turn, expression of cox15 was seen to increase the maximum viable cell numbers achieved relative to the control, and to a greater extent than cdkl3. Quantitatively similar results were obtained with two Human Embryonic Kidney-293 (HEK-293) cell lines and a Chinese Hamster Ovary (CHO) cell line. Additionally, HEK-293 cells secreting adipocyte complement-related protein of 30 kDa (acrp30) exhibited a slight increase in specific protein production and higher total protein production in response to the insertion of either cdkl3 or cox15. The effect of cdkl3 on cell growth is consistent with its homology to the cdk3 gene which is involved in G1 to S phase transition. Likewise, the increase in cell viability due to cox15 expression is consistent with its role in oxidative phosphorylation as an assembly factor for cytochrome c oxidase and its involvement removing apoptosis-inducing oxygen radicals. The use of microarray technology to identify genes influential to specific cellular processes raises the possibility of engineering cell lines as desired to meet production needs.

In addition, as described in Example 2, a Human Embryonic Kidney-293 (HEK-293) cell line initially propagated in 10% fetal bovine serum (FBS) was gradually adapted to serum free media and analyzed at specific serum levels using oligonucleotide microarrays. A number of genes were found to be differentially expressed several of which were then verified using RT-PCR. Two genes, egr1 and gas6, were selected for further analysis based on their level of differential expression, overall expression patterns, and proposed functionalities. HEK-293 cells propagated in 10% FBS were transfected with either egr1 or gas6 and then adapted to SFM. Another HEK-293 cell line constitutively secreting a protein, acrp30 (adipocyte complement-related protein of 30 kDa), was also transfected with either egr1 or gas6 and then adapted to SFM. Results indicated higher expression of either egr1 or gas6 enhanced the ability of both cell lines to adapt to serum free media by maintaining higher viability levels and improved growth rates at low serum levels. Egr1 appeared to have a greater impact on adaptability than gas6. In order to ascertain the effects, if any, egr1 had on protein production an ELISA for acrp30 was conducted. Results illustrated specific protein production was unaltered when the expression of egr1 was increased. In addition, flow cytometric experiments revealed increased expression of egr1 was closely correlated with an increase in the percentage of cells in the G0 phase.

The results described in Examples 9 and 10 were obtained using the following methods:

### Bioreactor Setup and Sampling

The two HeLa cell lines, attached and suspension were obtained from the American Type Culture Collection (ATCC, Manassas, Va.) (Catalog Nos. CCL-2 and CCL-2.2, respectively). Each cell line was grown in a BioFlo 3000 bioreactor (New Brunswick Scientific Co., Edison, N.J.) with a working volume of 1.5 L. Both cells lines were grown concurrently. Runs were conducted for up to 7 days after inoculation with constant sampling to characterize growth parameters. Three different runs were carried out for each cell line. The media used was DMEM (Biosource International, Camarillo, Calif.) supplemented with 10% FBS (Biosource International, Camarillo, Calif.). The attached HeLa cells were grown on Cytodex 3 (Amersham Biosciences, Piscataway, N.J.) microcarriers. Each reactor was seeded 5 with 2.0×10⁵ cells/mL. Cells used to seed the bioreactors were synchronized using serum deprivation for 24 hours (Merrill et al., Methods Cell Biol 1998, 57:229-249. Simon et al., Genet Epidemiol 2002, 23:21-26).

Each bioreactor was sampled at regular intervals to test media composition (i.e. pH, Glucose, Lactate), cell viability, and cell density. Each bioreactor was sampled for microarray analysis at the same time corresponding to different phases of growth under batch conditions. Samples were placed in 2 mL RNase/DNase-free micro tubes (Marsh Biomediacal Products, Rochester, N.Y.), combined with TRIzol reagent (Invitrogen, Carlsbad, Calif.) and stored at −80° C.

### Growth of HEK-293 Cells

HEK-293 cells (Catalog No. CRL-1573) were purchased from the American Type Culture Collection (ATCC, Manassas, Va.). These cells were initially grown using DMEM (Biosource International, Camarillo, Calif.) supplemented with 10% FBS (Biosource International, Camarillo, Calif.). The cells were grown in 75 cm2 and 162 cm2 T-flasks (Corning, Corning, N.Y.) as well as 250 mL spinner flasks (Bellco Glass, Vineland, N.J.) with Cytodex 3 microcarriers (GE Healthcare, Piscataway, N.J.). Cells were adapted to serum-free media

### RNA Isolation and Sample Preparation

Total RNA was isolated and purified from samples using an Invitrogen kit (Micro-to-Midi Total RNA Purification System) and quantified using a spectrophotometer, GeneQuant Pro (Biochrom Ltd, Cambridge, UK). Two absorbance values were determined for each sample assayed; one at 260 nm and the other at 280 nm. The quality of RNA was determined by the ratio of these numbers (A_{260/280}). Only samples with an A_{260/280} of at least 1.8 were subsequently used for microarray analysis (Simon et al., Genet Epidemiol 2002, 23:21-26). Each sample, consisting of approximately 10 μg of total RNA, was reverse transcribed, labeled, and prepared for microarray hybridization using a protocol from The Institute for Genomic Research (TIGR) in Rockville, Md.: “Aminoallyl labeling of RNA for Microarrays” (SOP # M004, Rev. 2) with an effective date of Mar. 4, 2002.

cDNA Microarray Analysis

Microscope slides (Corning, Corning, N.Y.) were printed with a set of 32,448 spots corresponding to approximately 14,000 unique genes by TIGR (Rockville, Md.) using an array fabricator (Intelligent Automation, Rockville, Md.). Microarray preparation and hybridization were performed using a protocol “Microarray labeled probe hybridization” (SOP # M005, Rev. 3) available from TIGR with an effective date of Sep. 11, 2002. After washing and drying, each slide was ready for scanning and image analysis using a GenePix 4000B (Molecular Devices Corporation, Sunnyvale, Calif.). Both technical and biological replicates were included in the microarray experiments. To ensure proper labeling efficiency the dyes for half of the arrays were swapped (Quackenbush, Nat Rev Genet. 2001, 2:418-427; Simon et al., Genet Epidemiol 2002, 23:21-26 43).

In other experiments, each RNA sample was reverse transcribed, labeled, and prepared for hybridization with Human Genome U133 Plus 2.0 Arrays (Affymetrix, Santa Clara, Calif.). Protocols were provided by Affymetrix and were specific for the arrays used. The hybridized arrays were scanned using a GeneChip Scanner 3000 (Affymetrix, Santa Clara, Calif.).

In some experiments, Acuity software (Molecular Devices Corporation, Sunnyvale, Calif.) was used to analysis the data starting with total intensity normalization (Burke, Mol Diagn 2000, 5:349-357). Additional steps were taken to filter the data, removing spots with either poor signal quality or genes with highly variable (i.e. inconsistent) expression ratios (Hegde et al., Biotechniques 2000, 29:548-550, 552-554, 556). In other experiments, two software programs, Acuity (Molecular Devices Corporation, Sunnyvale, Calif.) and Partek Pro (Partek Incorporated, St. Louis, Mo.) were used to analyze the data. Partek Pro was used to normalize and filter the data whereas Acuity was used to probe the data with clustering algorithms. Non-parametric normalization was applied to the data (Sidorov et al, 2002). Next, the data were filtered as part of a quality control step to remove spots with poor signal quality, defined as having low total signal or a signal below local background (Conway and Schoolnik, 2003).

The expression ratio for a gene, as referred to in the present study, is defined as the test sample intensity divided by the control sample intensity. The suspension HeLa cell line was considered the test whereas the attached HeLa cell line was considered the control. An expression ratio of unity indicates equal hybridization between the two samples being assayed whereas expression ratios above or below 1.0 are referred to as being upregulated or downregulated, respectively (Quackenbush, Nat Rev Genet. 2001, 2:418-427).

The clustering algorithms applied to the data included: self-organizing maps (SOMs), principle component analysis (PCA), and hierarchical clustering (Quackenbush, Nat Rev Genet. 2001, 2:418-427). Genes clustered together were probed to identify subsets of genes with relevance to cellular growth (i.e. cell cycle regulation, apoptosis, and/or signal transduction). The most differentially expressed were then identified and evaluated based on their known or proposed functionalities as indicated in the literature (Burke, Mol Diagn 2000, 5:349-35715).

Data files were exported into Acuity software where a number of clustering algorithms including self-organizing maps (SOMs) and principle component analysis (PCA) were applied (Quackenbush, Nat Rev Genet. 2001, 2:418-427). These algorithms were applied in order to identify genes with expression patterns closely correlated with decreasing serum levels. Genes that were identified in this manner were further probed based on gene ontology; searching specifically for relevance to cellular growth, survivability (i.e. resistance to apoptosis), and cell cycle regulation. Using this approach, two genes were selected for further investigation; egr1 and gash.

The microarray results were confirmed using reverse transcription-polymerase chain reaction (RT-PCR). The protocols used were provided by Applied Biosystems (4333458, Rev. B & 4335626, Rev. C, both dated May 2004). Each gene was assayed three times through two separate experiments to establish greater statistical significance.

Enhanced Expression of Genes in Various Cell Lines—cdkl3 and cox15

Sequencing information for both cdkl3 and cox15 were obtained using two public online databases; Harvester (European Molecular Biology Laboratory, Heidelberg, Germany) and GenBank (National Institutes of Health, Bethesda, Md.) (Harvester [http://harvester.fzk.de/harvester/;GenBank [http://www.ncbi.nlm.nih.gov/). For each gene, a plasmid containing the full-length gene was purchased from GeneCopoeia (Germantown, Md.). Each plasmid also contained the neomycin gene, conferring resistance to the compound geneticin (Invitrogen, Carlsbad, Calif.). This resistance allowed for the selection of clones expressing the plasmid in the mammalian cell lines examined (Bohm et al., Biotechnol Bioeng 2004, 88:699-706, Pollard Cell Biology. Philadelphia: Saunders; 2004). In subsequent experiments, cells transfected with empty plasmids (i.e. plasmids containing the neomycin gene and other components but neither genes of interest, cdkl3 or cox15) served as the control.

To check gene and protein sequence homologies the Basic Local Alignment Search Tool (BLAST) provided by the National Center for Biotechnology Information (NCBI) (Bethesda, Md.) was used. Based on these results, several cell lines were selected for investigation. Each of the following cell lines was purchased from ATCC (Manassas, Va.): HEK-293 (Catalog No. CRL-1573), CHO (Catalog No. CCL-61), and MDCK (Catalog No. CCL-34). Another cell line, HEK-293 ACRP30, was also studied because it constitutively expresses acrp30 for which an enzyme-linked immunosorbent assay (ELISA) is available (Mancia Structure 2004, 12:1355-1360, Berg Nat Med 2001, 7:947-953). Both HEK-293 cell lines were grown in DMEM (Biosource International, Camarillo, Calif.) with 10% FBS (Biosource International, Camarillo, Calif.). The media used with the CHO cells was F-12K (ATCC, Manassas, Va.) supplemented with 10% FBS (ATCC, Manassas, Va.). The MDCK cells were grown in EMEM (ATCC, Manassas, Va.) supplemented with 10% FBS (ATCC, Manassas, Va.). All of these cell lines were grown in an incubator (Thermo Scientific, Waltham, Mass.) set at 37° C. and 5% CO₂. In addition, all of the cell lines were dissociated using a trypsin-EDTA solution (ATCC, Manassas, Va.).

Once several different cell lines were selected for analysis, subsequent transfections were performed using manufacturer provided protocols packaged with each transfecting agent. For the attached HeLa cells and the HEK-293 cell lines (HEK-293 and HEK-293 ACRP30), the transfecting agent used was Lipofectamine 2000 (Invitrogen, Carlsbad, Calif.). In contrast, the transfecting agent used for the suspension HeLa cells and the CHO cells was Lipofectamine LTX (Invitrogen, Carlsbad, Calif.). And for the MDCK cells, Optifect (Invitrogen, Carlsbad, Calif.) was the transfecting agent used.

Transfections were performed in 24-well plates (Corning, Corning, N.Y.). Within 24 hours following the transfection the media was replaced with complete media. Once the cells had recovered, usually after 48 hours, geneticin was added to a final concentration of 750 μg/mL based on a prior kill curves (data not shown). Over several days, as media was periodically replaced, colonies began to form. Individual colonies were isolated and moved into 96-well plates (Corning, Corning, N.Y.). Upon reaching confluency, each well was expanded sequentially into 24, 12, and 6-well plates (Corning, Corning, N.Y.) before being counted and screened for expression based on western blot analysis. Physiological changes, post-transfection, were also observed using a DM IRB microscope and attached camera (Leica Camera, Allendale, N.J.).

Western blotting was conducted to verify translation of gene inserts via transfected plasmids. Protocols for this method were made available by the manufacturers of the primary antibodies for COX15 (Abnova, Taipei City, Taiwan) and CDKL3 (Abeam, Cambridge, Mass.). Briefly, cell lysate samples were prepared from a population of cells that were counted and assayed for viability. These samples were diluted with reducing sample buffer and separated using 4-20% Tris-Glycine gels (Invitrogen, Carlsbad, Calif.). Proteins were transferred to nitrocellulose membranes (Invitrogen, Carlsbad, Calif.) and blocked with blotting grade blocker, non-fat dry milk (Bio-Rad, Hercules, Calif.) for 1 hour. Membranes were then incubated with the primary antibodies at a dilution of 1:500 for 1 hour followed by incubation with secondary antibodies (HRP-conjugated secondary antibodies) for another hour. Incubation with a color development agent (KPL, Gaithersburg, Md.) allowed visualization of the blots which were then scanned.

Following clone screening, cells expressing the desired plasmid were expanded successively into 6-well plates and 25 cm² T-flasks. For growth comparisons, 75 cm² T-flasks (Corning, Corning, N.Y.) and 250 mL spinner flasks (Bellco Glass, Vineland, N.J.) were seeded and monitored for pH (Radiometer Analytical SAS, Lyon, France), cell viability & density (Cedex HiRes, Innovatis, Malvern, Pa.), and metabolite concentrations (YSI, Yellow Springs, Ohio) (Bleckwenn Biotechnol Bioeng 2005, 90:663-674; Merrill GV: Cell synchronization. Methods Cell Biol 1998, 57:229-249). In conjunction with the spinner flasks, Cytodex 3 microcarriers (GE Healthcare, Piscataway, N.J.) were used for the cell lines studied (Burke, Mol Diagn 2000, 5:349-357). Cells used for seeding were synchronized using the mitotic shake-off method (Merrill, Methods Cell Biol 1998, 57:229-249).

Modification of Gene Expression in Two HEK-293 Cell Lines and Adaptation—egr1 and gas6

Sequencing information for both egr1 and gas6 were obtained using two public online databases: Harvester (available on the world wide web at harvester.embl.de, European Molecular Biology Laboratory, Heidelberg, Germany) and GenBank (available on the world wide web at ncbi.nlm.nih.gov, National Institutes of Health, Bethesda, Md.). For each gene, a plasmid containing the full-length gene was purchased from GeneCopoeia (Germantown, Md.). Each plasmid also contained the neomycin gene, conferring resistance to the compound geneticin (Invitrogen, Carlsbad, Calif.). This resistance allowed for the selection of clones expressing the plasmid (Bohm et al., 2004). In subsequent experiments, cells transfected with empty plasmids (i.e. plasmids containing the neomycin gene and other components but neither genes of interest, egr1 or gas6) served as the control.

Another cell line, HEK-293 ACRP30, was also studied because it constitutively expresses acrp30 (adipocyte complement-related protein of 30 kDa) for which an enzyme-linked immunosorbent assay (ELISA) is available (Mancia et al. supra; Berg Nat Med 2001, 7:947-953). This cell line was also initially grown in DMEM (Biosource International, Camarillo, Calif.) with 10% FBS (Biosource International, Camarillo, Calif.).

Transfections were performed using manufacturer provided protocols packaged with the transfecting agent. For both HEK-293 cell lines the transfecting agent used was Lipofectamine 2000 (Invitrogen, Carlsbad, Calif.). Transfections were performed in 24-well plates (Corning, Corning, N.Y.). Within 24 hours following the transfection the media was replaced with complete media. Once the cells had recovered, usually after 48 hours, geneticin was added to a final concentration of 750 μg/mL based on a prior kill curves (data not shown). Over several days, as media was periodically replaced, colonies began to form. Individual colonies were isolated and moved into 96-well plates (Corning, Corning, N.Y.). Upon reaching confluency, each well was expanded sequentially into 24, 12, and 6-well plates (Corning, Corning, N.Y.) before being counted and screened for expression based on western blot analysis. Physiological changes, post-transfection, were also observed using a DM IRB microscope and attached camera (Leica Camera, Allendale, N.J.).

Western blotting was conducted to verify translation of gene inserts via transfected plasmids as described above.

Following clone screening, cells expressing the desired plasmid were expanded successively into 6-well plates and 25 cm² T-flasks. These cells were then adapted to serum-free media using a sequential method, previously described (Sinacore et al., 2000). Cells were expanded into 75 cm² T-flasks (Corning, Corning, N.Y.) and 250 mL spinner flasks (Bellco Glass, Vineland, N.J.). Samples were taken regularly and assayed for pH (Radiometer Analytical SAS, Lyon, France), cell viability & density (Cedex HiRes, Innovatis, Malvern, Pa.), and metabolite concentrations (YSI, Yellow Springs, Ohio).

### Flow Cytometry, ELISA and Caspase Assay

Cells grown in 162 cm² T-flasks were sampled at different points along their respective growth curves for analysis using a flow cytometer. Samples were taken during the early, middle, and late exponential growth phase, following synchronization by the mitotic shake-off method (Merrill, supra). In other experiments, Cells synchronized using the mitotic shake-off method, were used to seed 75 cm² T-flasks (Merrill, supra Davis Biotechniques 2001, 30:1322-1326). These flasks were sampled for analysis using a flow cytometer. Flow cytometry experiments were performed using a CyAn LX flow cytometer (DakoCytomation, Fort Collins, Colo.). Essentially, cells were fixed with ethanol and stained with propidium iodide (Becton Dickinson, Franklin Lakes, N.J.) at a concentration between 0.5−1.0×10⁶ cells/mL (Berg Nat Med 2001, 7:947-953). Summit software (DakoCytomation, Fort Collins, Colo.) was used to capture data generated by the sampled cells such as the level of light scattering and fluorescence. In contrast, Modfit software (Molsoft, La Jolla, Calif.) was used to identify varying stages of the cell cycle and calculate the percentages of cells in each stage.

A kit produced by R&D Systems (Catalog No. MRP300, Minneapolis, Minn.) was used to perform the ELISA which quantified the amount of acrp30 secreted by HEK-293 ACRP30 cells. A microplate spectrofluorometer (SpectraMax Gemini XS, Molecular Devices, Sunnyvale, Calif.) was used to quantify the amount of protein present in the samples. Initially, samples were run at varying dilutions to establish the minimum dilution level necessary to obtain values that fall within the standard curve. This value was determined to 1:4,000 (data not shown).

### Other Embodiments

From the foregoing description, it will be apparent that variations and modifications may be made to the invention described herein to adopt it to various usages and conditions. Such embodiments are also within the scope of the following claims.

All patents and publications mentioned in this specification are herein incorporated by reference to the same extent as if each independent patent and publication was specifically and individually indicated to be incorporated by reference.

1. A method for identifying a gene whose expression modulates cellular adhesion, the method comprising:
(a) comparing the expression of a gene in an anchorage-dependent cell relative to an anchorage-independent cell; and
(b) identifying a gene that is differentially expressed in the anchorage-dependent cell relative to the anchorage-independent cell, wherein an alteration in the level of gene expression between the cells identifies a gene whose expression modulates cellular adhesion.

2. The method of claim 1, wherein the expression of the gene is increased or decreased in the anchorage-dependent or the anchorage-independent cell.

3. The method of claim 1, wherein the method further comprises the steps of
(c) altering the expression of the gene in the cell, and
(d) comparing the adhesion, proliferation, mortality, or other growth characteristic of the cell relative to a corresponding control cell.

4-8. (canceled)

9. The method of claim 3, wherein the gene is selected from the group consisting of cdkl3, siat7e, lama4, cox15, egr1, gash, map3k9, and gap43.

10. A method for modulating a cell growth characteristics, the method comprising contacting the cell with an agent that increases or decreases the expression of a gene identified according to the method of claim 1 in a cell, thereby modulating a cell growth characteristics.

11. A method for modulating recombinant polypeptide expression in a cell, the method comprising contacting a cell that expresses a recombinant polypeptide with an agent that increases or decreases the expression of a gene identified according to the method of claim 1 or a gene selected from the group consisting of cdkl3, siat7e, lama4, cox15, egr1, gash, map3k9, and gap43 in a cell, thereby modulating expression of the recombinant polypeptide.

12-13. (canceled)

14. A method for modulating cellular mortality, the method comprising contacting the cell with an agent that increases or decreases the expression of egr1 or gash or a gene identified according a previous aspect in a cell, thereby modulating cellular mortality.

15. The method of claim 1, wherein the agent is an expression vector that encodes the gene.

16-18. (canceled)

19. A method for increasing cell adhesion, the method comprising:
(a) contacting a cell with an expression vector comprising a nucleic acid molecule that encodes lamanin α4; and
(b) increasing lamanin α4 expression in the cell, thereby increasing cell adhesion.

20. The method of claim 19, wherein the method increases laminin α4 transcription or translation in the cell.

21-22. (canceled)

23. A method for decreasing cell adhesion, the method comprising contacting a cell expressing lamanin α4 with an agent that decreases lamanin α4 expression or activity in the cell, thereby decreasing cell adhesion.

24. The method of claim 22, wherein the agent is a lamanin α4 inhibitory nucleic acid molecule.

25. (canceled)

26. A method for decreasing cell adhesion, the method comprising:
a) contacting a cell with an expression vector comprising a nucleic acid molecule that encodes sialyltransferase 7E; and
(b) increasing sialyltransferase 7E expression in the cell, thereby decreasing cell adhesion.

27. The method of claim 26, wherein the method increases the transcription or translation of a sialyltransferase 7E nucleic acid molecule in the cell.

28. The method of claim 25, wherein the expression vector comprises a constitutive or conditional promoter operably linked to a sialyltransferase 7E nucleic acid molecule.

29. (canceled)

30. A method for increasing cell adhesion, the method comprising contacting a cell expressing sialyltransferase 7E with an agent that decreases sialyltransferase 7E expression or activity in the cell, thereby increasing cell adhesion.

31-32. (canceled)

33. A method for increasing the expression of a recombinant polypeptide in a cell, the method comprising:
(a) contacting a cell that expresses a recombinant polypeptide with an expression vector comprising a nucleic acid molecule or an inhibitory nucleic acid molecule selected from the group consisting of cdkl3, siat7e, lama4, cox15, egr1, gas6, map3k9, and gap43; and
(b) altering expression of the nucleic acid molecule in the cell, thereby increasing the expression of the recombinant polypeptide in the cell.

34-35. (canceled)

36. A method for increasing the expression of a recombinant polypeptide in a cell, the method comprising:
(a) contacting a cell that expresses a recombinant polypeptide with an expression vector comprising a nucleic acid molecule encoding a polylaminin α4 or sialyltransferase 7E; and
(b) increasing laminin α4 or sialyltransferase 7E expression in the cell, thereby increasing the expression of the recombinant polypeptide in the cell.

37. A method for altering the growth characteristics of a cell, the method comprising:
(a) contacting the cell with an expression vector encoding a polypeptide selected from the group consisting of siat7e, lama4, cdkl3, cox15, egr1, and gas6 polypeptide; and
(b) expressing the polypeptide in the cell, thereby altering the cell's growth characteristics.

38. (canceled)

39. An expression vector comprising:
a nucleic acid molecule encoding a polypeptide selected from the group consisting of cdkl3, siat7e, lama4, cox15, egr1, gas6, map3k9, and gap43; or
(ii) a nucleic acid molecule encoding an inhibitory nucleic acid molecule complementary to a gene selected from the group consisting of cdkl3, siat7e, lama4, cox15, egr1, gas6, map3k9, and gap43.

40-42. (canceled)

43. An inhibitory nucleic acid molecule that reduces the expression of a nucleic acid molecule selected from the group consisting of cdkl3, siat7e, lama4, cox15, egr1, gas6, map3k9, and gap43.

44. (canceled)

45. A cell comprising an expression vector of claim 39, or a cell comprising a mutation that alters the expression or activity of a polypeptide selected from the group consisting of cdkl3, siat7e, lama4, cox15, egr1, gas6, map3k9, and gap43 polypeptide.

46-56. (canceled)
